(19) United States
(12) Patent Application Publication
(10) Pub. No.: US 2026/0193677 A1
(43) Pub. Date: Jul. 9, 2026

(54) METHODS OF GENOME EDITING OOCYTES

(71) Applicant: The Regents of the University of California, Oakland, CA (US)

(72) Inventors: Alison Louise Van EENENNAAM, Davis, CA (US); Jason LIN, Davis, CA (US)

(21) Appl. No.: 18/870,297

(22) PCT Filed: Jun. 2, 2023
(86) PCT No.: PCT/US2023/067885

§ 371 Date: Nov. 27, 2024

Related U.S. Application Data
(60) Provisional application No. 63/348,885, filed on Jun. 3, 2022.
(60) Provisional application No. 63/459,195, filed on Apr. 13, 2023.

(51) Int. Cl. C12N 15/873; A01K 67/0278; A61D 19/04; C12N 9/22; C12N 13/00; C12N 15/11; C12N 15/86; C12N 15/87; C12N 15/90

(52) U.S. Cl. CPC C12N 15/873; A01K 67/0278; A61D 19/04; C12N 9/226; C12N 13/00; C12N 15/11; C12N 15/86; C12N 15/87; C12N 15/907; A01K 2217/052; A01K 2217/072; A01K 2227/101; A01K 2227/103; C12N 2310/20; C12N 2750/14143

(57) ABSTRACT

Provided in this disclosure are methods of producing a fertilized embryo comprising a genomic edit and engineered cells and organisms therefrom. In certain aspects, engineered cells can be created utilizing AAVs and CRISPR/Cas systems.

### CROSS-REFERENCE TO RELATED APPLICATIONS

This application is a US National Phase Application Under 371 of International Application No. PCT/US2023/067885 filed Jun. 2, 2023, which claims priority to, and the benefit of, co-pending U.S. Provisional Application No. 63/348,885, entitled “METHODS OF GENOME EDITING OOCYTES” filed on Jun. 3, 2022; and U.S. Provisional Application No. 63/459,195, entitled “METHODS OF GENOME EDITING OOCYTES” filed on Apr. 13, 2023, the entire contents of which are incorporated by reference in their entireties as if fully set forth herein.

### STATEMENT AS TO RIGHTS TO INVENTIONS MADE UNDER FEDERALLY SPONSORED RESEARCH AND DEVELOPMENT

This invention was made with government support under Grant No. 2020-67015-31536 awarded by the National Institute of Food and Agriculture. The government has certain rights in the invention.

### REFERENCE TO A “SEQUENCE LISTING,” A TABLE, OR A COMPUTER PROGRAM LISTING APPENDIX SUBMITTED ON A COMPACT DISK

The instant application contains a Sequence Listing that has been filed electronically in .xml format and is hereby incorporated by reference in its entirety. Said .xml copy, created on Jun. 2, 2023, is named “UC_2022_608_3_1387487_Sequence_Listing.xml” and is 97 kb in size.

### BACKGROUND

Genetic improvement of livestock species is critical for increasing the efficiency of animal production and improving wellbeing. However, traditional breeding approaches for genetic improvement of livestock are typically slow due to the long generation interval of large livestock species, and introgression of traits from one breed to another often results in linkage drag. Genome editing technologies offer an approach to introduce targeted genetic alterations in livestock genomes to augment traditional selective breeding approaches. Typically, large (>1 kb) targeted insertions or knock-ins (KI) using genome editing have been achieved by introducing editing reagents and homology directed repair (HDR) nucleic acid templates into somatic cell lines, followed by somatic cell nuclear transfer (SCNT) cloning. This constrains the genetic diversity of the resultant animals to that of the cell lines. Additionally, the efficiency of cloning in large livestock species is low and perinatal abnormalities are common. Alternatively, genome editing reagents can be introduced into mammalian zygotes using cytoplasmic or pronuclear microinjection. This time-consuming procedure requires expensive equipment and a high level of technical skill, rendering it unscalable and inaccessible for laboratories without specialized equipment or personnel.

Electroporation is a widely used technique for delivering drugs and nucleic acids into living cells. Electroporators work by directing “poring” pulses of electrical current to create transient (msec to minute range) pores in the lipid bilayer of the plasma membrane which allows the passage of reagents into the cell. Whereas SCNT and microinjection require the operator to manipulate each zygote individually and precisely, electroporation allows for the simultaneous and instantaneous processing of upwards of 100 zygotes with the push of a button, making it a scalable and simple approach to producing genome edited livestock.

Genome-edited mice, rats, and pigs have been produced through the electroporation of early-stage embryos. There are a handful of papers describing the electroporation of bovine embryos to generate small mutations, however no papers have reported on the introduction of large (>1 kb) targeted insertions using electroporation. Zygotes are enclosed in a hard glycoprotein matrix, the zona pellucida. Large HDR DNA templates are unable to pass this physical barrier without further manipulation. This complicates the production of gene-edited animals harboring useful exogenous genes as HDR templates containing a gene and promoter often result in DNA cassettes that are larger than 1 kb. Previous efforts to produce mammalian embryos harboring targeted insertions of more than 1 kb have required either the removal of the zona pellucida or microinjection of donor template prior to electroporation. Removal of the zona pellucida prior to electroporation requires a strict protocol that results in sticky and damaged embryos which become difficult to work with, while the microinjection of the donor template prior to electroporation defeats the purpose of using electroporation as an scalable and high-throughput approach to generating genome edited animals.

Thus, there is a need for improved methods for introducing genetic changes, particular genetic insertions such as gene knock-ins, into zygotes.

### BRIEF SUMMARY

Described herein are methods of producing a fertilized embryo comprising a genomic edit. Methods as described herein can comprises providing isolated mature cumulus-oocyte complexes (COC) from a first non-human organism; removing the cumulus cells from the isolated mature COC to produce denuded oocytes; incubating the denuded oocytes with sperm, adeno-associated virus (AAV) particles comprising a donor nucleic acid having homology to a nucleotide sequence adjacent to a target genomic site, and supplemental mature COC from a second non-human organism to produce a first plurality of cells comprising at least one zygote comprising the donor nucleic acid; removing the cumulus cells from the first plurality of cells thereby producing a plurality of denuded cells comprising at least one denuded zygote; electroporating the plurality of denuded cells in the presence of a site-directed nuclease that binds to the target genomic site; and culturing the electroporated plurality of denuded cells to produce at least one fertilized embryo comprising the genomic edit.

In some embodiments, the AAV particles can be AAV1, AAV2, AAV3, AAV4, AAV5, AAV6, AAV7, AAV8, or AAV9 particles, or any combination of any thereof. In some embodiments, the AAV particles can be AAV6 particles. In some embodiments, the AAV particles can be recombinant AAV particles.

In some embodiments, the donor nucleic acid can be a portion of a donor template. In some embodiments, the donor template can be part of a plasmid or linear nucleic acid.

In some embodiments, step c) can comprise incubating the denuded oocytes and the supplemental mature COC at an approximate ratio of about 4:1.

In some embodiments, the incubating of step c) can be performed for about 6 hours.

In some embodiments, the site-directed nuclease can be a Cas protein. In some embodiments, the Cas protein can be selected from the group consisting of Cas5, Cas6, Cas7, Cas8, Cas9, Cas12a, Cas12b, Cas12i, Cas12j, Cas12L, Cas12e, Cas12c, Cas12d, Cas12g, Cas12h, TnpB, Cas13a, Cas13b, Cas14, and nickase or deactivated versions thereof, or any combination of any thereof. In some embodiments, the Cas protein can be a Cas9 enzyme. In some embodiments, the Cas protein can be a Cas12a enzyme. In some embodiments, the Cas protein can comprise a nuclear localization signal (NLS). In some embodiments, the Cas protein can be part of a ribonucleoprotein (RNP), wherein the RNP can comprise the Cas protein bound to a guide RNA comprising a nucleotide sequence having complementarity to a binding site at the target genomic site.

In some embodiments, the genomic edit can be an excision, an insertion, or a replacement of at least a portion of the target genomic site. In some embodiments, the genomic edit can be a gene knock-in at the target genomic site.

In some embodiments, the first non-human organism and the second non-human organism can be the same organism. In some embodiments, the first non-human organism and the second non-human organism can be different organisms.

In other aspects, described herein are fertilized embryos comprising one or more genomic edits per embryo. In another embodiment, provided herein are fertilized embryos comprising the genomic edit produced by any of the methods as describe herein.

In additional aspects, described herein are non-human organisms comprising one or more genomic edits. In another embodiment, provided is a non-human organism comprising the genomic edit developed from the at least one fertilized embryo.

In additional aspects, described herein are methods of producing a non-human organism comprising one or more genomic edits. In another embodiment, provided is a method of producing a non-human organism comprising the genomic edit comprising implanting the at least one fertilized embryo into a female non-human organism for gestation.

### BRIEF DESCRIPTION OF THE DRAWINGS

An understanding of the features and advantages of the present disclosure will be obtained by reference to the following detailed description that sets forth illustrative embodiments, in which the principles of the present disclosure may be utilized, and the accompanying drawings.

FIGS. 1A and 1B: Mutation efficiency (%) for NGS barcode matched reads from individual electroporated blastocysts for (A) bovine (n=109) and (B) ovine (n=38) embryos. Samples with <100 reads were removed from the data set. NGS, next generation sequencing.

FIG. 2 is a graphical NGS representation of the 30 most common allelic variants for barcode-unmatched reads from electroporated bovine blastocysts. There were 11.4% of reads with wild-type sequence.

FIG. 3 is a graphical NGS representation of the 30 most common allelic variants for barcode-unmatched reads from electroporated ovine blastocysts. There were 5.3% of reads with wild-type sequence.

FIGS. 4A-4C are representative images of bovine blastocysts after transduction with rAAV6 serotype carrying the GFP reporter gene at a concentration of 1010 vgc using (A) an FITC filter, (B) bright field, and (C) merged.

FIG. 5 is an image of bovine blastocysts that underwent rAAV6 incubation and electroporation imaged on day 7 postfertilization using an FITC filter. Blastocyst did not express GFP, however, cumulus cells that remained in culture expressed GFP.

FIG. 6 is a graph showing KI efficiency (% of blastocysts) and blastocyst development rate (% of oocytes that developed into blastocysts) for denuded oocytes transduced with rAAV6 at concentrations of 7×10¹⁰, 8×10¹⁰, 9×10¹⁰, and 10¹¹ vgc, and blastocyst development rate (% of oocytes that developed into blastocysts, green) for nontransduced, nondenuded control for each experiment. The number of observations for each category is shown at the bottom of the bar. KI, knock-in. Left bars represents knock-in efficiency; middle bars represent treated blastocysts; right bars represent control blastocysts.

FIG. 7 is an image of PCR genotyping results of a treated GFP-expressing blastocyst (lanes 1-2), untreated wild-type blastocyst (lanes 3-4), treated granulosa cell DNA (lanes 5-6), water (lanes 7-8), and untreated wild-type blastocyst (lane 9). Lanes 1-8 were PCR amplified using primers flanking the 5′ (bH11LjuncF2, bH11LjuncR2; 1083 bp) and 3′ (bH11RjuncF2, bH11RjuncR2; 1444 bp) junctions of the targeted KI. The treated GFP blastocyst and treated cell DNA had confirmed targeted KIs as seen in lanes 1-2 and 5-6. The wild-type blastocyst and water as shown in lanes 3-4 and 7-8 did not harbor the targeted KIs, as expected. Lane 9 was PCR amplified using primers bH11WTF2, bH11WTR2 targeting the bovine genome outside of the homology-directed repair template shows the wild-type H11 sized amplicon (1547 bp) as expected. PCR, polymerase chain reaction.

FIGS. 8A-8C are images of GFP-expressing bovine blastocyst that underwent rAAV6 incubation and electroporation imaged on day 7 postfertilization using (A) a FITC filter, (B) bright field, and (C) merged (bright field+FITC).

FIG. 9 is an illustration the CMV-eGFP reporter plasmid according to the present disclosure. The location of PCR primers aavGFPF2 and aavGFPR2 are noted thereon.

FIG. 10 shows a schematic of a 3.9 kb HDR donor template containing 600 bp H11 homology arms with gRNA target sites at the ends, the CAG promoter, GFP gene with a nuclear localization signal, and rAAV2 ITR arms according to various embodiments of this disclosure.

FIG. 11 shows a workflow used to test rAAV serotypes 1, 2, 5, 6, 8, and 9 packaged with the CMV-eGFP reporter plasmid for transduction efficiency at various concentrations according to various embodiments of this disclosure.

FIG. 12 is an illustration of an embodiment of a workflow to produce transfected and edited bovine blastocysts according to the present disclosure.

FIG. 13 is an illustration of another embodiment of a workflow to produce transfected and edited bovine blastocysts according to the present disclosure that is modified from other embodiments disclosed herein, namely FIGS. 11 and 12.

FIGS. 14A-14E shows the design of PCR primers used in the Examples of this disclosure. If there is a knock-in, left junction primers (bH11LjuncF2, bH11LjuncR2) result in a 1083 bp amplicon and right junction primers (bH11RjuncF2, bH11RjuncR2) result in a 1444 bp amplicon. Sanger sequencing for both the left and right junctions confirmed the presence of a targeted knock-in. Chromatograms for a GFP expressing bovine blastocyst at the left junction and right junction are shown. Conversely, wild-type genotype at the H11 locus in the bovine genome using primers bH11WTF2/bH11WTR2 result in a 1547 bp amplicon.F

FIG. 15 is an image of a gel electrophoresis run showing PCR products of a primary line of granulosa cells lipofected with the rAAV6 plasmid containing the HDR donor template (FIG. 9) and Cas9:sgRNA RNP targeting the H11 locus (Lanes 2-5). Lane 2: primers flanking the H11 target site (H11F2/H11R2), and lane 3: primers that amplified from outside the homology arms (bH11LjuncF2/bH11RjuncR2). The presence of cells with no targeted knock-in was detected by the presence of wildtype-sized bands, 505 bp and 1954 bp, respectively. Lane 4: primers flanking the left junction (bH11LjuncF2, bH11LjuncR2; 1083 bp) of the targeted knock-in, and lane 5: primers targeting GFP (aaVGFPF/aaVGFPF; 560 bp). Lanes 6-9 included control bovine genomic DNA template amplified with primer sets in the same order as for the granulosa cells. Lanes 1 and 10 are 1 kb+ ladder (Invitrogen, Track-it 1 kb plus).

FIGS. 16A-16B are images of a representative PCR gels of blastocyst following two rounds of DNA amplification using left junction primers (bH11LjuncF2, bH11LjuncR2; FIG. 16A), and right junction primers (bH11RjuncF2, bH11RjuncR2; FIG. 16B) indicating a targeted gene knock-in. Lane 1 is 1 kb+ ladder (Invitrogen, Track-it 1 kb plus) in both gels. Lanes 4,7,8,9 were positive for the left junction amplicon 1,083 bp and lanes 2, 5 and 10 for the right junction amplicon.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting” of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the combination[s] of features in the claims or other aspects of the present disclosure. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less' and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

The recitation of numerical ranges by endpoints includes all numbers and fractions subsumed within the respective ranges, as well as the recited endpoints.

### I. General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E.A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); Marten H. Hofker and Jan van Deursen, Transgenic Mouse Methods and Protocols, 2^{nd} edition (2011); and Primrose and Twyman. Principles of Gene Manipulation and Genomics 2006, published by Blackwell Publishers.

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7^{th} Ed. 2021, published by McGraw Hill; Isik et la., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a” “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform according to the present disclosure. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

As used herein, the terms “encoding” or “encoded”, with respect to a specified nucleic acid, is meant comprising the information for translation into the specified protein. A nucleic acid encoding a protein can comprise intervening sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. When the nucleic acid is prepared or altered synthetically, advantage can be taken of known codon preferences of the intended host where the nucleic acid is to be expressed. As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form or are not natively found operatively linked to each other in the species. A heterologous protein may originate from a foreign species or, if from the same species, is substantially modified from its original form by deliberate human intervention.

As used herein, the term “recombinant” or “engineered” can generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, “culturing” can refer to maintaining cells under conditions in which they can proliferate and avoid senescence as a group of cells. “Culturing” can also include conditions in which the cells also or alternatively differentiate. Culturing can include one or more steps or conditions, and include in one or more steps passaging, transfer of cells, media changing, incubation temperature changes, atmospheric gas changes, and/or the like.

As used herein, a “population” of cells is any number of cells greater than 1, but is preferably at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

As used herein, “gene product” refers to any polynucleotide that is transcribed (in vivo or in vitro) into an RNA molecule. The term “gene product” also refers to polypeptides that are translated from an RNA gene product.

As used herein, “polypeptides” or “proteins” refers to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

The term “fragment” as used herein with reference to a nucleic acid (polynucleotide) generally denotes a 5′- and/or 3′-truncated form of a nucleic acid. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the nucleic acid sequence length of said nucleic acid. For example, insofar not exceeding the length of the full-length nucleic acid, a fragment may include a sequence of ≥5 consecutive nucleotides, or ≥10 consecutive nucleotides, or ≥20 consecutive nucleotides, or ≥30 consecutive nucleotides, e.g., ≥40 consecutive nucleotides, such as for example ≥50 consecutive nucleotides, e.g., ≥60, ≥70, ≥80, ≥90, ≥100, ≥200, ≥300, ≥400, ≥500 or ≥600 consecutive nucleotides of the corresponding full-length nucleic acid. The terms encompass fragments arising by any mechanism, in vivo and/or in vitro, such as, without limitation, by alternative transcription or translation, exo- and/or endo-proteolysis, exo- and/or endo-nucleolysis, or degradation of the peptide, polypeptide, protein, or nucleic acid, such as, for example, by physical, chemical and/or enzymatic proteolysis or nucleolysis.

As used herein, “fragment” as used herein with reference to a peptide, polypeptide, or protein generally denotes a portion of the peptide, polypeptide, or protein, such as typically an N- and/or C-terminally truncated form of the peptide, polypeptide, or protein. Preferably, a fragment may comprise at least about 30%, e.g., at least about 50% or at least about 70%, preferably at least about 80%, e.g., at least about 85%, more preferably at least about 90%, and yet more preferably at least about 95% or even about 99% of the amino acid sequence length of said peptide, polypeptide, or protein. For example, insofar not exceeding the length of the full-length peptide, polypeptide, or protein, a fragment may include a sequence of ≥5 consecutive amino acids, or ≥10 consecutive amino acids, or ≥20 consecutive amino acids, or ≥30 consecutive amino acids, e.g., ≥40 consecutive amino acids, such as for example ≥50 consecutive amino acids, e.g., ≥60, ≥70, ≥80, ≥90, ≥100, ≥200, ≥300, ≥400, ≥500 or ≥600 consecutive amino acids of the corresponding full-length peptide, polypeptide, or protein.

As used herein, “expression” refers to the process by which polynucleotides are transcribed into RNA transcripts. In the context of mRNA and other translated RNA species, “expression” also refers to the process or processes by which the transcribed RNA is subsequently translated into peptides, polypeptides, or proteins. In some instances, “expression” can also be a reflection of the stability of a given RNA. For example, when one measures RNA, depending on the method of detection and/or quantification of the RNA as well as other techniques used in conjunction with RNA detection and/or quantification, it can be that increased/decreased RNA transcript levels are the result of increased/decreased transcription and/or increased/decreased stability and/or degradation of the RNA transcript. One of ordinary skill in the art will appreciate these techniques and the relation “expression” in these various contexts to the underlying biological mechanisms.

As used herein “reduced expression” or “underexpression” refers to a reduced or decreased expression of a gene or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual. By way of a non-limiting example, if said sample is a sample of a lung tumor and comprises lung tissue, said control is lung tissue of a healthy individual. The term “reduced expression” preferably refers to at least a 25% reduction, e.g., at least a 30%, 40%, 50%, 60%, 70%, 75%, 80%, 85%, 90%, 95%, 98% or 99% reduction, relative to such control.

The term “modification causing said reduced expression” refers to a modification in a gene which affects the expression level of that or another gene such that the expression level of that or another gene is reduced or decreased. Said modification can be any nucleic acid modification including, but not limited to, a mutation, a deletion, an insertion, a replacement, a ligation, a digestion, a break and a frameshift. Said modification is preferably selected from the group consisting of a mutation, a deletion and a frameshift. In particular embodiments, the modification is a mutation which results in reduced expression of the functional gene product.

As used herein “increased expression” or “overexpression” are both used to refer to an increased expression of a gene or gene product thereof in a sample as compared to the expression of said gene or gene product in a suitable control. The term “increased expression” preferably refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control.

The term “modification causing said increased expression” refers to a modification in a gene which affects the expression level of that or another gene such that expression of that or another gene is increased. Said modification can be any nucleic acid modification including, but not limited to, a mutation, a deletion, an insertion, a replacement, a ligation, a digestion, a break and a frameshift. Said modification is preferably selected from the group consisting of a mutation, a deletion and a frameshift. In particular embodiments, the modification is a mutation which results in reduced expression of the functional gene product.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “targeting moiety” refers to molecules, complexes, agents, and the like that is capable of specifically or selectively interacting with, binding with, acting on or with, or otherwise associating or recognizing a target molecule, agent, and/or complex that is associated with, part of, coupled to, another object, complex, surface, and the like, such as a cell or cell population, tissue, organ, subcellular locale, object surface, particle etc. Targeting moieties can be chemical, biological, metals, polymers, or other agents and molecules with targeting capabilities. Targeting moieties can be amino acids, peptides, polypeptides, nucleic acids, polynucleotides, lipids, sugars, metals, small molecule chemicals, combinations thereof, and the like. Targeting moieties can be antibodies or fragments thereof, aptamers, DNA, RNA such as guide RNA for a RNA guided nuclease or system, ligands, substrates, enzymes, combinations thereof, and the like. The specificity or selectivity of a targeting moiety can be determined by any suitable method or technique that will be appreciated by those of ordinary skill in the art. In some embodiments, the targeting moiety has a specificity the equilibrium dissociation constant, Kd, is 10⁻³ M or less, 10-4 M or less, 10⁻⁵ M or less, 10⁻⁶ M or less, 10⁻⁷ M or less, 10-8 M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, the targeting moiety has increased binding with, association with, interaction with, activity on as compared to non-targets, such as a 1 to 500 (or more) fold increase. Targets of targeting moieties can be amino acids, peptides, polypeptides, nucleic acids, polynucleotides, lipids, sugars, metals, small molecule chemicals, combinations thereof, and the like. Targets can be receptors, biomarkers, transporters, antigens, complexes, combinations thereof, and the like.

As used herein, “wild-type” is the average form of an organism, variety, strain, gene, protein, or characteristic as it occurs in a given population in nature, as distinguished from mutant forms that may result from selective breeding, recombinant engineering, and/or transformation with a transgene.

As used herein, a “biological sample” refers to a sample obtained from, made by, secreted by, excreted by, or otherwise containing part of or from a biologic entity (i.e. an individual). A biologic sample can contain whole cells and/or live cells and/or cell debris, and/or cell products, and/or virus particles. The biological sample can contain (or be derived from) a “bodily fluid.” The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a bovine. Mammals include, but are not limited to, murines, simians, humans, farm animals, sport animals, and pets. These terms include non-human organisms. Tissues, cells, and their progeny of a biological entity obtained in vivo or cultured in vitro are also encompassed.

As used herein, “self-renewing” refers to the capacity of an undifferentiated cell to divide while maintaining an undifferentiated state in at least one of the progeny cells so as maintain or expand the undifferentiated cell population, while optionally give rise to a differentiated cell or cell population. Thus, “self-renewing cells”, as the term is used herein, are undifferentiated cells that have the capacity to divide and optionally differentiate, where upon division, at least one of the progeny cells retain an undifferentiated state so as to allow for maintenance or expansion of the undifferentiated cell population.

As used herein, “totipotent” refers to the capacity of a cell or cell population to differentiate into any cell type (e.g., of a blastomere) or a complete embryo or animal (inclusive of a placenta). Thus, “totipotent cells”, as the term is used herein, are cells that have the capacity to differentiate into or give rise to any cell type (e.g., of a blastomere) or a complete embryo or animal (inclusive of a placenta). In other words, totipotent cells can develop a complete organism on their own. For example, zygotes are totipotent. Totipotent cells have the capacity to divide until the entire embryo or animal is formed.

As used herein, an “oocyte” is an immature egg (an immature ovum).

As used herein, “blastocyst” means an early developmental stage of embryo comprising of inner cell mass (from which embryo proper arises) and a fluid filled cavity typically surrounded by a single layer of trophoblast cells. “Developmental Biology”, sixth edition, ed. by Scott F. Gilbert, Sinauer Associates, Inc., Publishers, Sunderland, Mass. (2000).

“Recombination” is the exchange of DNA strands to produce new nucleotide sequence arrangements. The term may refer to the process of homologous recombination that occurs in double-strand DNA break repair, where a polynucleotide is used as a template to repair a homologous polynucleotide. The term may also refer to exchange of information between two homologous chromosomes during meiosis.

“Homology dependent repair” or “homology directed repair” or “HDR” refers to a mechanism for repairing ssDNA and double stranded dna (dsDNA) damage in cells. This repair mechanism can be used by the cell when there is an HDR template with a sequence with significant homology to the injury site. The term “perfect HDR” refers to a situation in which genomic-homology junctions in the replaced allele underwent complete HDR and “imperfect HDR” refers to a situation in which genomic-homology junctions in the replaced allele underwent partial or incomplete HDR. a donor DNA molecule with homology to the cleaved target DNA sequence is used as a template for repair of the cleaved target DNA sequence, resulting in the transfer of genetic information from the donor polynucleotide to the target DNA. As such, new nucleic acid material may be inserted/copied into the site. In some cases, a target DNA is contacted with a donor molecule, for example a donor DNA molecule. In some cases, a donor DNA molecule is introduced into a cell. In some cases, at least a segment of a donor DNA molecule integrates into the genome of the cell.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the present disclosure. For example, in the appended claims, any of the claimed embodiments can be used in any combination of any element therein.

### II. Overview

Provided in this disclosure are methods of producing a fertilized embryo comprising a genomic edit and engineered cells and organisms therefrom. Transfer of large (>1 kb) nucleic acid templates into zygotes is hindered by the zona pellucida, which the provided method overcomes. As described herein, the inventors have developed a scalable approach to deliver homology directed repair (HDR) donor repair templates of up to 4.7 kb, along with genome editing reagents into zygotes, using recombinant adeno-associated viruses (rAAVs) in combination with electroporation. As described in the Examples of this disclosure, the inventors first tested a panel of eight natural rAAV serotypes (1, 2, 5, 6, 8, and 9) packaged with a CMV-eGFP reporter for transduction efficiency at various concentrations, and then packaged a 3.9 kb homology directed repair (HDR) template into the most efficient serotype, rAAV6. The rAAV6 GFP HDR repair template was then incubated with matured and denuded bovine oocytes at various concentrations for 6 hours during fertilization. To facilitate fertilization, cumulus oocyte complexes (COC) were incubated together with the matured and denuded bovine oocytes, sperm, and AAV particles. The presumptive zygotes were then electroporated to transfect a sgRNA/CAS9 ribonucleoprotein (RNP) complex targeting the H11 locus. This method resulted in knock-in and blastocysts rates of 35.7% (n=14), 6.7% (n=209) for rAAV6 at 8×10¹⁰ genomic copies (GC); 26.3% (n=19), 8.6% (n=222) for rAAV6 at 9×10¹⁰ GC; and 23.1% (n=13), 3.7% (n=351) for rAAV6 at 10¹¹ GC, respectively.

As described in this disclosure, the inventors determined that electroporation of bovine and ovine zygotes according to the methods provided herein resulted in the efficient production of genome-edited blastocysts. It was demonstrated that a targeted 2.7 kb knock-in (KI) in bovine embryos can be achieved using the combination of rAAV to deliver a 3.9 kb HDR donor template and electroporation to deliver the Cas9:sgRNA RNP editing reagents 6 h post-insemination. With this approach, there was no need to remove or weaken the zona pellucida (ZP) and, of the blastocysts that developed, a KI rate of up to approximately 38% was observed.

The holy grail of livestock editing is an approach to edit embryos efficiently in a commercial setting and in a way that avoids mosaicism. Electroporation methods as described in this disclosure go partway toward that goal in that electroporation can be used to efficiently introduce targeted deletions in zygotes and it removes the need for micromanipulation equipment and a trained operator for gene-editing reagents to be introduced into each zygote individually. The pairing of rAAV with electroporation provides an approach to additionally transduce HDR templates of up to 4.9 kb across the ZP into zygotes along with editing reagents.

### III. Methods

### A. Producing Genetically Modified Embryos

In one aspect, provided is a method of producing a fertilized embryo comprising a genomic edit. In some embodiments, the method comprises providing isolated mature cumulus-oocyte complexes (COC) from a first non-human organism. The method further comprises removing the cumulus cells from the isolated mature COC to produce denuded oocytes. In some instances, a portion of the mature COC is set aside and is not denuded. In some instances, COC are obtained from the ovaries of a first non-human organism and allowed to mature in vitro for approximately 24 hours. Typical maturation conditions are described, e.g., in Bakhtari, A. & Ross, P. J. (2014) DPPA3 prevents cytosine hydroxymethylation of the maternal pronucleus and is required for normal development in bovine embryos. Epigenetics 9, 1271-1279; DOI: 10.4161/epi.32087. The oocytes are denuded using standard techniques, such as vortexing the COC.

In some embodiments, the method further comprises producing a first plurality of cells comprising at least one zygote comprising a donor nucleic acid by incubating the denuded oocytes with sperm, adeno-associated virus (AAV) particles comprising a donor nucleic acid having homology to a nucleotide sequence adjacent to a target genomic site, and supplemental mature COC from a second non-human organism. In some embodiments, the first non-human organism and the second non-human organism are the same organism. For example, in some instances, the supplemental mature COC are the portion of the initially provided mature COC that was set aside and not denuded. In some embodiments, the first non-human organism and the second non-human organism are different organisms. In some embodiments, the method further comprises removing the cumulus cells from the first plurality of cells thereby producing a plurality of denuded cells comprising at least one denuded zygote.

In some embodiments, the denuded oocytes, sperm, AAV particles, and supplemental mature COC are incubated for at least 1 hour, 2 hours, 3 hours, 4 hours, 4.5 hours, 5 hours, 5.5 hours, 6 hours, or 6.5 hours. In some embodiments, the incubation is performed for 5.5-6.5 hours. In some embodiments, the incubation is performed for 6 hours. The transduction and fertilization incubation is performed under typical cell culture conditions for mammalian fertilization (e.g., 38.5° C. in a humidified atmosphere of 5% carbon dioxide, 5% oxygen, and 90% nitrogen). Typical fertilization conditions are described, e.g., in Owen, J. R., et al. (2021). One-step generation of a targeted knock-in calf using the CRISPR-Cas9 system in bovine zygotes. BMC Genomics 22(1): 118; DOI: 10.1186/s12864-021-07418-3 and “In Vitro Production of Bovine Embryos,” P.J. Hansen Laboratory, Department of Animal Sciences, University of Florida, version Nov. 17, 2017, available at animal.ifas.ufl.edu/hansen/ivf_docs/University %20of %20Florida %20Bovine %20IVP %20Manua 1%20ver0% 2011.17.2017.pdf.

In some instances, the fertilization and transduction step is performed using 10, 15, 20, 25, 20, 35, 40, 45, 50, 55, 60, 65, 70, 75, 80, 85, 90, or 100 mature and denuded oocytes. In some instances, the fertilization and transduction step is performed using approximately 20-30 mature and denuded oocytes. In some instances, sperm added to the culture medium comprises a concentration of 1-2 million sperm/mL. In some instances, 10⁵-10⁷ sperm are added per 25 oocytes; for example, 10⁵, 5×10⁵, 10⁶, 5×10⁶, 10⁷, or 5×10⁷. In some instances, 10⁶ sperm are added per 25 oocytes. In some instances, 50,000-100,000 sperm are added per 25 oocytes.

In some embodiments, the ratio of denuded oocytes to supplemental mature COC is 1:1, 1.5:1, 2:1, 2.5:1, 3:1, 3.5:1, 4:1, 4.5:1, 5:1, 5.1:1, or 6:1. In some embodiments, the ratio of denuded oocytes to supplemental mature COC is 3:1 to 5:1. In some embodiments, the ratio of denuded oocytes to supplemental mature COC is 3.5:1 to 4.5:1. In some embodiments, the ratio of denuded oocytes to supplemental mature COC is approximately 4:1. In some embodiments, the ratio of denuded oocytes to supplemental mature COC is 4:1. In some embodiments, the presence of the cumulus cells in the supplemental nature COC during the incubation facilitates fertilization of the denuded oocytes such that the fertilization rate of the denuded oocytes in the presence of the supplemental mature COC is greater than the fertilization rate of denuded oocytes in the absence of the supplemental mature COC as discussed in the Examples of this disclosure.

The donor nucleic acid is introduced into the plurality of denuded cells, particularly into at least one zygote produced during the above-referenced incubation step, by transduction by a viral, pseudoviral, and/or virus like particle, such as an AAV particle. Methods of packaging the genetic modifying systems and/or components thereof in viral particles can be accomplished using any suitable viral vector or vector systems. Such viral vector and vector systems are described in greater detail elsewhere herein. As used in this context herein “transduction” refers to the process by which foreign nucleic acids and/or proteins are introduced to a cell (prokaryote or eukaryote) by a viral, pseudoviral, and/or virus like particle. After packaging in a viral, pseudoviral, and/or virus like particle. the viral particles can be exposed to cells (e.g., in vitro, ex vivo, or in vivo) where the viral, pseudoviral, and/or virus like particle infects the cell and delivers the cargo to the cell via transduction. Viral, pseudoviral, and/or virus like particles can be optionally concentrated prior to exposure to target cells. In some embodiments, the virus titer of a composition containing viral and/or pseudoviral particles can be obtained and a specific titer be used to transduce cells. Viral vectors and systems and generation of viral (or pseudoviral, and/or virus like particle) delivery particles is described in greater detail elsewhere herein. Viral transduction has been used to deliver exogenous nucleic acid constructs to bovine cells. See e.g., Hoffmann et al., Biology of Reproduction, Vol. 71, Issue 2, 1 Aug. 2004, pag. 405-409, doi.org/10.1095/biolreprod.104.028472; Yu et al., (2014) Expression of Intracellular Interferon-Alpha Confers Antiviral Properties in Transfected Bovine Fetal Fibroblasts and Does Not Affect the Full Development of SCNT Embryos. PLoS ONE 9(7): e94444. doi.org/10.1371/journal.pone.0094444; and Wu et al., Scientific Reports, Vol. 6, Article No. 28343 (2016), which are incorporated by reference as if expressed in their entireties herein and can be adapted for use with the present disclosure.

Recombinant adeno associated viruses (rAAV) have been employed to deliver nucleic acids to various cell types for many years. They are non-pathogenic nature, can package either single or double stranded DNA, and have been shown to efficiently transduce various mammalian cell types [21-23]. The genome of wild type adeno-associated viruses contains only four genes (rep, cap, aap, maap) flanked by inverted terminal repeats (ITRs) on both sides. The rep gene is required for viral genome replication and packaging, the cap gene produces viral capsids, the aap gene promotes capsid assembly, and the maap gene helps facilitate viral replication [23, 24]. Conversely, rAAV does not contain viral DNA and only requires the presence of 130 bp AAV ITR arms flanking a DNA fragment of up to 4.7 kb on either side for packaging [25]. The ITRs are the only cis-acting components necessary for the packaging and replication of DNA fragments [26]. Thus, rAAV can deliver HDR templates of up to 4.7 kb to facilitate gene knock ins.

In some embodiments, the AAV particles are AAV1, AAV2, AAV3, AAV4, AAV5, AAV6, AAV7, AAV8, or AAV9 particles. In some embodiments, the AAV particles are AAV6 particles. In some embodiments, the AAV particles are recombinant AAV particles. Additional details relating to use of AAV is described below.

The amount of AAV particles incubated with the mature and denuded oocytes, sperm, and supplemental COC can be measured in terms of viral genome copies (vgc or GC). In some instances, AAV particles can be included in the incubation step at 5×10¹⁰ vgc, 6×10¹⁰ vgc, 7×10¹⁰ vgc, 8×10¹⁰ vgc, 9×10¹⁰ vgc, 1×10¹¹ vgc, 2×10¹¹ vgc, or 3×10¹¹ vgc.

In some embodiments, the plurality of denuded cells are then electroporated in the presence of a site-directed nuclease that binds to the target genomic site. Electroporation uses pulsed high-voltage electrical currents to transiently open nanometer-sized pores within the cellular membrane of cells suspended in buffer, allowing for components with hydrodynamic diameters of tens of nanometers to flow into the cell. In this instance, electroporation is used for delivery of the site-directed nuclease into the plurality of denuded cells, particularly into the at least one denuded oocyte. Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391. Electroporation has been used to deliver exogenous polynucleotides and/or polypeptides to bovine zygotes. See e.g., Lin and Van Eenennaam. Front Genet. 2021; 12: 648482, doi.org/10.3389/fgene.2021.648482 (particularly at supplementary table 1).

In some embodiments, the voltage and number of pulses for delivery of an exogenous polynucleotide to a bovine cell, such as a zygote or blastocyst, via electroporation is 10-20 V/mm and 2-6 pulses, 10-20V/mm and 2-3 pulses, 15-20V/mm and 2-3 pulses, 15V/mm and 6 pulses See e.g., Tanihara, F., Hirata, M., Morikawa, S., Nguyen, N. T., Le, Q. A., Hirano, T., et al. (2019). The effects of electroporation on viability and quality of in vivo-derived bovine blastocysts. J. Reprod. Dev. 65, 475-479. doi: 10.1262/jrd.2019-049; Namula, Z., Wittayarat, M., Hirata, M., Hirano, T., Nguyen, N. T., Le, Q. A., et al. (2019). Genome mutation after the introduction of the gene editing by electroporation of Cas9 protein (GEEP) system into bovine putative zygotes. In Vitro Cell. Dev. An. 55, 598-603; Miao, D., Giassetti, M. I., Ciccarelli, M., Lopez-Biladeau, B., and Oatley, J. M. (2019). Simplified pipelines for genetic engineering of mammalian embryos by CRISPR-Cas9 electroporation dagger. Biol. Reprod. 101, 177-187; Ciccarelli, M., Giassetti, M. I., Miao, D., Oatley, M. J., Robbins, C., Lopez-Biladeau, B., et al. (2020). Donor-derived spermatogenesis following stem cell transplantation in sterile NANOS2 knockout males. Proc. Natl. Acad. Sci. U.S.A 117, 24195-24204; Camargo, L. S. A., Owen, J. R., Van Eenennaam, A. L., and Ross, P. J. (2020). Efficient one-step knockout by electroporation of ribonucleoproteins into zona-intact bovine embryos. Front. Genet. 11:570069; and Wei, J., Gaynor, P., Cole, S., Brophy, B., Oback, B., and Laible, G. (2018). “Developing the laboratory conditions for bovine zygote-mediated genome editing by electroporation” in Proceedings of the World Congress on Genetics Applied to Livestock Production, which are incorporated by reference herein and can be adapted for use with the present disclosure. In some embodiments, electroporation conditions can comprise 20 volts, 3 bipolar pulses, 3.5 msec pulse length, 50 msec intervals, 0% decay rate. In some embodiments, electroporation can be performed on plurality of denuded cells comprising 30-100 cells.

In some embodiments, the electroporated plurality of denuded cells, which comprise at least one denuded zygote, are then cultured to produce at least one fertilized embryo comprising the genomic edit. The culturing step is performed under typical cell culture conditions for blastocyst development (e.g., 38.5° C. in a humidified atmosphere of 5% carbon dioxide, 5% oxygen, and 90% nitrogen). Typical culturing conditions are described, e.g., in Owen, J. R., et al. (2021). One-step generation of a targeted knock-in calf using the CRISPR-Cas9 system in bovine zygotes. BMC Genomics 22(1): 118; DOI: 10.1186/s12864-021-07418-3. The culturing step is performed for at least 5 days, 6 days, or 7 days. In some instances, the culturing step is performed for 7 days, at which point zygotes typically reach the blastocysts stage. In some instances, the plurality of denuded cells is cultured until the at least one denuded zygote matures to a blastocyst.

The presence of the donor template and site-directed nuclease in the at least one denuded zygote results in the genomic edit being made at the target genomic site in the genome of the at least one denuded zygote. The genomic edit can be an excision, an insertion, or a replacement of at least a portion of the target genomic site. In some instances, the genomic edit is a knock-in of a gene sequence at the target genomic site. In some instances, the genomic edit is a knock-out of a gene at the genomic target site.

A “donor nucleic acid,” “donor polynucleotide”, “donor molecule”, or “donor template” is a nucleotide polymer or oligomer intended for insertion at a target polynucleotide, typically a target genomic site. The donor sequence may be one or more transgenes, expression cassettes, or nucleotide sequences of interest. A donor molecule may be a donor DNA molecule, either single stranded, partially double-stranded, or double-stranded. The donor polynucleotide may be a natural or a modified polynucleotide, a RNA-DNA chimera, or a DNA fragment, either single- or at least partially double-stranded, or a fully double-stranded DNA molecule, or a PGR amplified ssDNA or at least partially dsDNA fragment. In some embodiments, the donor DNA molecule is part of a circularized DNA molecule. In some instances, a fully double-stranded donor DNA can provide increased stability as dsDNA fragments are generally more resistant than ssDNA to nuclease degradation.

The donor molecule may comprise at least 10 contiguous nucleotides (often referred to as a homology arm), wherein the nucleic acid molecule is at least 70% identical to a genomic nucleotide sequence, such that these contiguous nucleotides are sufficient for homologous recombination of the donor DNA molecule into the genome of the cell at the targeted genomic DNA sequence following cleavage, e.g., by a site-directed nuclease. In some embodiments, the donor DNA molecule can comprise at least about 10, 20, 30, 50, 70, 80, 100, 150, 200, 250, 300, 250, 400, 450, 500, 600, 700, 800, 900, 1000, or 1500 nucleotides, including any value within this range not explicitly recited herein, wherein the donor DNA molecule is at least 70%, 75%, 80%, 85%, 90%, 91%, 92%, 93%, 94%, 95%, 96%, 97%, 98%, 99%, or 100% identical to a genomic nucleic acid sequence. In some embodiments, the donor DNA molecule may be substantially complementary to a genomic nucleic acid sequence. In some embodiments, the donor DNA molecule comprises heterologous nucleic acid sequence. In some embodiments, the donor DNA molecule comprises at least one expression cassette. In some embodiments, the donor DNA molecule may comprise a transgene, which comprises at least one expression cassette. In some embodiments, the donor DNA molecule comprises an allelic modification of a gene which is native to the target genome. The allelic modification can comprise at least one nucleotide insertion, at least one nucleotide deletion, and/or at least one nucleotide substitution. In some embodiments, the allelic modification can comprise a small insertion or deletion. In some embodiments, the donor DNA molecule comprises homologous arms to the target genomic site. In some embodiments, the donor DNA molecule comprises at least 100 contiguous nucleotides at least 90% identical to a genomic nucleic acid sequence, and optionally may further comprise a heterologous nucleic acid sequence such as a transgene.

The donor polynucleotide may be any suitable nucleic acid. In some embodiments, the donor nucleic acid is a portion of a donor template. In some embodiments, the donor template is part of a plasmid or linear nucleic acid. In some embodiments, the donor nucleic acid is a portion of a chromosome.

In some embodiments, the denuded zygotes are electroporated in the presence of a site-directed nuclease or variant thereof. Site-directed nucleases (SDNs) (e.g. zinc finger nucleases, transcription activator-like effector nucleases, CRISPR-associated nucleases) have gained increasing popularity in the gene editing space act as endonucleases and generally create double-stranded breaks (DSBs) in specific DNA sequences, activating intrinsic repair mechanisms of the cell (e.g., homologous recombination). During the repair process, site-directed modification to said specific DNA sequence can be achieved. Site-directed nucleases are further discussed in Section III.C of this disclosure.

A site-directed nuclease cleaves target DNA. In some embodiments, a site-directed nuclease interacts with a guide RNA, which is either a single RNA molecule or a RNA duplex of at least two RNA molecules, and is guided to a DNA sequence by virtue of its association with the guide RNA. In some embodiments, the site-directed nuclease is able to cleave one or both strands of DNA at a specified target sequence. In some embodiments, the site-directed nuclease is a Cas protein. In some embodiments, the Cas protein is selected from the group consisting of Cas5, Cas6, Cas7, Cas8, Cas9, Cas12a, Cas12b, Cas12i, Cas12j, Cas12L, Cas12e, Cas12c, Cas12d, Cas12g, Cas12h, TnpB, Cas13a, Cas13b, Cas14, and nickase or deactivated versions thereof. In some embodiments, the Cas protein is a Cas9 enzyme. In some embodiments, the Cas protein is a Cas12a enzyme. In some embodiments, the Cas protein comprises a nuclear localization signal. In some embodiments, the Cas protein is part of a ribonucleoprotein (RNP), the RNP comprising the Cas protein bound to a guide RNA comprising a nucleotide sequence having complementarity to a binding site at the target genomic site.

In some instances, the at least one fertilized embryo comprising the genomic edit can be implanted into a recipient female non-human organism for gestation into and birth of an engineered non-human organism comprising the genomic edit. A typical implantation method is described, e.g., in Owen, J. R., et al. (2021). One-step generation of a targeted knock-in calf using the CRISPR-Cas9 system in bovine zygotes. BMC Genomics 22(1): 118; DOI: 10.1186/s12864-021-07418-3. Such organisms are described in more detail below.

### B. Vectors and Vector Systems

In some embodiments the delivery vehicle is a vector or vector system or particle, such as a virus or viral like particle, produced from such a vector or vector system. As such, also provided herein are vectors that can contain one or more of the genetic modifying system polynucleotides described herein. In certain embodiments, the vector can contain one or more polynucleotides encoding one or more elements of a genetic modifying system described herein. The vectors can be useful in producing bacterial, fungal, yeast, plant cells, animal cells, and transgenic animals that can express one or more components of the genetic modifying system described herein, and as such, contain a genetic modification or be rendered capable of producing particles (e.g., viral or viral like particles) that can be used to deliver a genetic modifying system described herein to a cell, such as a bovine cell.

Within the scope of this disclosure are vectors containing one or more of the polynucleotide sequences of interest, such as those useful for introducing an insertion or gene knock-in or other genomic edit into a host cell. One or more of the polynucleotides that are part of a genetic modifying system can be included in a vector or vector system. The vectors and/or vector systems can be used, for example, to express one or more of the polynucleotides in a cell, such as a producer cell, to produce a genetic modifying system containing virus particles. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the present disclosure in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In some embodiments, the vector can be a bicistronic vector. In some embodiments, a bicistronic vector can be used for one or more elements of the genetic modifying system described herein.

In some embodiments, expression of elements of the genetic modifying system described herein can be driven by the CBh promoter or other ubiquitous promoter. In some embodiments, the promoter is a bovine promoter. In some embodiments, the promoter is a late spermatogenesis promoter. Exemplary late spermatogenesis promoters are described elsewhere herein.

Where the element of the genetic modifying system is an RNA, its expression can be driven by a Pol III promoter, such as a U6 promoter. In some embodiments, the two are combined.

### 1. Cell-Based Vector Amplification and Expression

Vectors may be introduced and propagated in a prokaryotic cell or eukaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In some embodiments, a prokaryote is used to amplify copies of a vector and express one or more nucleic acids, such as to provide a source of one or more proteins for delivery to a host cell or host organism.

Vectors can be designed for introduction into a suitable host cell. In some embodiments, the suitable host cell is a prokaryotic cell. Suitable host cells include, but are not limited to, bacterial cells, yeast cells, insect cells, and mammalian cells. In some embodiments, the suitable host cell is a eukaryotic cell. In some embodiments the host cell is a cell to be modified by a genetic modifying system. In some embodiments the host cell is a producer cell capable of producing particles (e.g., virus particles, virus like particles, exosomes, and/or the like) that can be used to deliver a genetic modifying system or component thereof to a cell.

In some embodiments, the suitable host cell is a suitable bacterial cell. Suitable bacterial cells include but are not limited to bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U2OS, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif. (1990). In some embodiments, the suitable host cell is a bovine cell, including but not limited to, bovine embryonic stem cells, bovine induced pluripotent stem cells, bovine blastocyst cells, bovine spermatogonia stem cells, bovine oogonial cells, bovine primordial germ cells, bovine primordial germ cell like cells, bovine totipotent cells, or other bovine cell described elsewhere herein.

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2 plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. In some embodiments, the suitable host cell is an insect cell. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements is provided elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET lid (Studier et al., GENE EXPRESSION TECHNOLOGY: METHODS IN ENZYMOLOGY 185, Academic Press, San Diego, Calif. (1990) 60-89).

In some embodiments, one or more vectors are introduced into a host cell to facilitate a genomic edit at one or more target sites on a target polynucleotide, such as in a target cell or target cell genome. For example, a donor sequence comprising a nucleic acid sequence of interest (e.g., a gene sequence of interest) can be operably linked to a regulatory element on a vector. In another example, different donor sequences comprising different nucleic acid sequences of interest (e.g., gene sequences of interest) can be operably linked to different regulatory elements on different vectors.

### 2. Vector Features

The vectors can include additional features that can confer one or more functionalities to the vector, the polynucleotide to be delivered (e.g. donor nucleic acid), a virus or other particle (e.g., viral like particle or exosome) produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### 3. Regulatory Elements

In certain embodiments, the vectors described herein can include one or more regulatory elements that can be operatively linked to a polynucleotide (e.g., donor nucleic acid) in a donor template. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization or export signals). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). Exemplary promoters also include bovine U6 (bU6) and bovine 7SK (b7SK), and other bovine PolII promoters (see e.g., Lambeth et al., Anim Genet. 2006 August; 37(4):369-72), bovine papillomavirus-1 promoters (BPV-1) (Linz and Baker. J Virol. 1988 August; 62(8):2537-43. doi: 10.1128/JVI.62.8.2537-2543.1988), the bovine SIX1 gene promoter (see e.g., Wei et al. Scientific Reports volume 7, Article number: 12599 (2017)), bovine growth hormone promoter (see e.g., Jiang et al., Nuc Acid Prot Syn Mol Gen. 1999. 274(12): 7893-7900), bovine pyruvate carboxylase (see e.g., Hazelton et al. J. Dairy Sci. 91:91-99), a bidirectional promoter (see e.g., Meersserman et al. DNA Research, Volume 24, Issue 3, June 2017, Pages 221-233), a bovine Akt3 promoter (see e.g., Farmanullah et al. Journal of Genetic Engineering and Biotechnology (2021) 19:164), bovine alpha-lactalbumin promoter (see e.g., FEBS Lett. 1991 Jun. 17; 284(1):19-22), bovine beta-casein promoter (see e.g., Cerdan et al., Mol Reprod Dev. 1998 March; 49(3):236-45), any combination thereof.

In some embodiments, the regulatory sequence can be a regulatory sequence described in U.S. Pat. No. 7,776,321, U.S. Pat. Pub. No. 2011/0027239, or International Patent Publication No. WO 2011/028929, the contents of which are incorporated by reference herein in their entireties. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. As used herein, “regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TNI3 (cTn1), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Ferll4), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g. myostatin, Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g. a promoter that is repressed (e.g. bound by a repressor) until the repressor condition of the promotor is removed (e.g. inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome)., such as a Light Inducible Transcriptional Effector (LITE) that direct changes in transcriptional activity in a sequence-specific manner. The components of a light inducible system may include one or more elements of the CRISPR-Cas system described herein, a light-responsive cytochrome heterodimer (e.g., from Arabidopsis thaliana), and a transcriptional activation/repression domain. In some embodiments, the vector can include one or more of the inducible DNA binding proteins provided in International Patent Publication No. WO 2014/018423 and U.S. Patent Publication Nos., 2015/0291966, 2017/0166903, 2019/0203212, which describe e.g., embodiments of inducible DNA binding proteins and methods of use and can be adapted for use with the present disclosure.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize 1n2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters that are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments where multiple elements are to be expressed from the same vector or within the same vector system, different promoters or regulatory elements can be used for each element to be expressed to avoid or limit loss of expression due to competition between promoters and/or other regulatory elements.

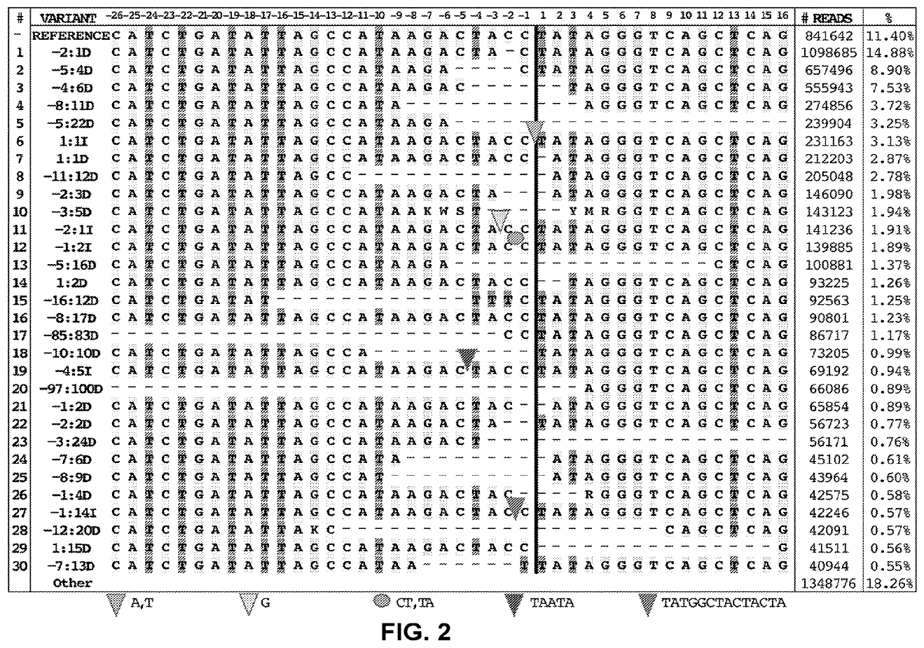

In some embodiments, the polynucleotide, vector or system thereof can include one or more elements capable of translocating and/or expressing a polynucleotide to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., genome.unmc.edu/LocSigDB/and Negi et al., 2015. Database. 2015: bav003; doi: 10.1093/database/bav003), nuclear export signals (e.g., LXXXLXXLXL (SEQ ID NO: 2) and others described elsewhere herein), endoplasmic reticulum localization/retention signals (e.g., KDEL (SEQ ID NO: 3), KDXX, KKXX, KXX, and others described elsewhere herein; and see e.g., Liu et al. 2007 Mol. Biol. Cell. 18(3):1073-1082 and Gorleku et al., 2011. J. Biol. Chem. 286:39573-39584), mitochondria targeting signals (see e.g., Chin, R. M., et al, 2018, Cell Reports. 22:2818-2826, particularly at FIG. 2; Doyle et al. 2013. PLoS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430, and peroxisome targeting signals (e.g. (S/A/C)-(K/R/H)-(L/A), SLK, (R/K)-(L/V/I)-XXXXX-(H/Q)-(L/A/F). Suitable protein targeting motifs can also be designed or identified using any suitable database or prediction tool, including but not limited to Minimotif Miner (minimotifminer.org, mitominer.mrc-mbu.cam.ac.uk/release-4.0/embodiment.do?name=Protein %20MTS), LocDB (see above), PTSs predictor, TargetP-2.0 (www.cbs.dtu.dk/services/TargetP/), ChloroP (www.cbs.dtu.dk/services/ChloroP/); NetNES (www.cbs.dtu.dk/services/NetNES/), Predotar (urgi.versailles.inra.fr/predotar/), and SignalP (www.cbs.dtu.dk/services/SignalP/).

### 4. Selectable Markers and Tags

A polynucleotide of interest (e.g., the donor nucleic acid) in the donor template described herein can be operably linked, fused to, or otherwise modified to include a polynucleotide that encodes or is a selectable marker or tag, which can be a polynucleotide or polypeptide. In some embodiments, the polypeptide encoding a polypeptide selectable marker is incorporated in the polynucleotide of the interest (e.g., donor nucleic acid) such that the selectable marker polypeptide, when translated, is inserted between two amino acids between the N- and C-terminus of the encoded polypeptide or at the N- and/or C-terminus of the encoded polypeptide. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the genetic modifying system (or other polynucleotide) described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as 0-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the genetic modifying system (or other polypeptide) described herein via suitable linker, such as a glycine or glycine serine linkers as short as GS or GG up to (GGGGG)₃ (SEQ ID NO: 4) or (GGGGS)₃ (SEQ ID NO: 5). Other suitable linkers are described elsewhere herein.

### 5. Targeting Moieties

The vector or vector system (or other polynucleotide) can include one or more polynucleotides that are or encode one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the genetic modifying system polynucleotide(s) and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated genetic modifying system polynucleotide(s) to specific cells, tissues, organs, etc. In some embodiments, the targeting moieties can target integrins on cell surfaces. Optionally, the binding affinity of the targeting moiety is in the range of 1 nM to 1 μM.

### 6. Codon Optimization

The polynucleotide of interest (e.g., donor nucleic acid) can encode a polypeptide of interest (e.g., a transgene)) that has been codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000,” Nucl. Acids Res. 28:292 (2000). Computer algorithms for codon optimizing a particular sequence for expression in a particular host cell are also available, such as Gene Forge (Aptagen; Jacobus, PA), are also available.

The vector polynucleotide can be codon optimized for expression in a specific cell-type, tissue type, organ type, and/or subject type, such as a bovine cell. In some embodiments, a codon optimized sequence is a sequence optimized for expression in a eukaryote, e.g., bovines (i.e., being optimized for expression in a bovine or bovine cell), or for another eukaryote, such as another animal (e.g., an ovine). Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g. astrocytes, glial cells, Schwann cells etc.), muscle cells (e.g. cardiac muscle, smooth muscle cells, and skeletal muscle cells), connective tissue cells (fat and other soft tissue padding cells, bone cells, tendon cells, cartilage cells), blood cells, stem cells (including embryonic stem cells, primordial germ cells, primordial germ cell like cells, pluripotent stem cells, totipotent stem cells, blastocysts, etc.) and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a mammal, including but not limited to human, or non-human eukaryote or animal or mammal as discussed herein, e.g., a bovine, ovine, camelid, and/or the like.

### 7. Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vectors described herein.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

### 8. Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as a donor nucleic acid (e.g., a donor template), into a virus particle and producing said virus particle when used alone or with one or more other viral vectors (such as in a viral vector system). Viral vectors and systems thereof can be used for producing viral particles for delivery of a donor nucleic acid. The viral vector can be part of a viral vector system involving multiple vectors. In some embodiments, systems incorporating multiple viral vectors can increase the safety of these systems. In some embodiments, the viral vectors are configured to produce replication incompetent viral particles for improved safety of these systems.

In certain embodiments, the virus structural component, which can be encoded by one or more polynucleotides in a viral vector or vector system, comprises one or more capsid proteins including an entire capsid. In certain embodiments, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the present disclosure can comprise one or more of VP1, and/or one or more of VP2, and/or one or more of VP3. Accordingly, the present disclosure is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Thus, a virus of within the family Adenoviridae is contemplated as within the present disclosure with discussion herein as to adenovirus applicable to other family members. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cell, glioblastoma cells, coronary artery endothelial cells and keratinocytes. See, e.g., Buning et al, 2015, Current Opinion in Pharmacology 24, 94-104. And as to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

### 9. Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments the AAV can integrate into a specific site on chromosome 19 of a human cell with no observable side effects. In some embodiments, the capacity of the AAV vector, system thereof, and/or AAV particles can be up to about 4.7 kb.

The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some embodiments, the AAV capsid can contain 60 capsid proteins. In some embodiments, the ratio of VP1:VP2:VP3 in a capsid can be about 1:1:10.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E40RF6, and VA RNAs. In some embodiments, a producing host cell line expresses one or more of the adenovirus helper factors.

The AAV vector or system thereof can be configured to produce AAV particles having a specific serotype.

AAV particles may comprise or be derived from any natural or recombinant AAV serotype. According to the present disclosure, the AAV particles may utilize or be based on a serotype selected from any of the following serotypes, and variants thereof including but not limited to AAV1, AAV10, AAV106.1/hu.37, AAV11, AAV114.3/hu.40, AAV12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LKO9, AAV-LK10, AAV-LK 11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh.13, AAVrh.13R, AAVrh.14, AAVrh.17, AAVrh.18, AAVrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AAVrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, BNP61 AAV, BNP62 AAV, BNP63 AAV, bovine AAV, caprine AAV, Japanese AAV 10, true type AAV (ttAAV), UPENN AAV 10, AAV-LK16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, and/or AAV SM 10-8. In other examples the AAV may be engineered as a hybrid AAV from two or more parental serotypes.

In some embodiments, the AAV vector or system thereof is configured as a “gutless” vector. In some embodiments, the “gutless” AAV vector or system thereof can have the cis-acting viral DNA elements involved in genome amplification and packaging in linkage with the heterologous sequences of interest (e.g., the genetic modifying system polynucleotide(s)).

AAV can be packaged as single-stranded (ssAAV) or self-complementary (scAAV) forms. The wild-type AAV genome is a linear single-stranded DNA (ssDNA) with two inverted terminal repeats (ITRs) forming a hairpin structure on each end. It is therefore also known as ssAAV. An scAAV vector, sometimes called dsAAV, contains complementary sequences that are capable of spontaneously annealing, upon infection, which eliminates the requirement for host cell DNA synthesis. scAAV vectors thus have smaller packaging capacity.

In some embodiments, the AAV vectors are produced in in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, an AAV vector or vector system can contain or consist essentially of one or more polynucleotides encoding one or more donor nucleic acid or other exogenous polynucleotide to be delivered to a cell. Specific cassette configuration for delivery of the polynucleotide(s) will be appreciated by one of ordinary skill in the art in view of the description herein.

### 10. Virus Particle Production From AAV

General principles of rAAV production are reviewed in, for example, Carter, 1992, Current Opinions in Biotechnology, 1533-539; and Muzyczka, 1992, Curr. Topics in Microbial. and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

In general, there are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the cargo polynucleotide to be packaged and delivered by the resulting AAV particle (e.g., the genetic modifying system polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a cargo polynucleotide (e.g., the CRISPR-Cas system polynucleotide(s)) between 2 ITRs; (2) a vector that carries the AAV Rep-Cap encoding polynucleotides; and (helper polynucleotides). One of skill in the art will appreciate various methods and variations thereof that are both helper and -helper free and as well as the different advantages of each system. See also Kimur et al., 2019. Sci. Rep. 6:13601; Shin et al., Meth. Mol Biol. 2012. 798:267-284; Negrini et al., 2020. Curr. Prot. Neurosci. 93:e103; Dobrowsky et al., 2021. Curr. Op. Biomed. Eng. 20:100353 for additional methods and techniques for AAV vector and particle production, which can be adapted for use with the present disclosure.

### C. Exemplary Genetic Modification Systems

As discussed above, in some embodiments, a site-directed nuclease is used to introduce the genomic edit in the at least one denuded zygote comprising the donor nucleic acid. The site-directed nuclease can be a component of a genetic modification system. In certain embodiments, the genetic modification system includes a programmable nuclease system (e.g., a CRISPR (or CRISPR-Cas) system), a zinc finger nuclease (ZFN) system, a TALEN, a meganuclease), a transposon system, recombinase, homing endonuclease, viral vector system, or any combination thereof. Various genetic modification systems have been used to modify bovine cells and/or generate modified bovines, including CRISPR-Cas systems, ZFNs, TALENs, and transposon systems. See e.g., Owen et al., BMC Genomics volume 22, Article number: 118 (2021); Yum S Y, Lee S J, Kim H M, Choi W J, Park J H, Lee W W, et al. Efficient generation of transgenic cattle using the DNA transposon and their analysis by next-generation sequencing. Sci Rep. 2016; 6(27185); Garrels W, Talluri T R, Apfelbaum R, Carratala Y P, Bosch P, Potzsch K, et al. One-step multiplex Transgenesis via sleeping beauty transposition in cattle. Sci Rep. 2016; 6(21953); Ding S, Wu X, Li G, Han M, Zhuang Y, Xu T. Efficient transposition of the piggyBac (PB) transposon in mammalian cells and mice. Cell. 2005; 122:473-83; Li T, Shuai L, Mao J, Wang X, Wang M, Zhang X, et al. Efficient production of fluorescent transgenic rats using the piggyBac transposon. Sci Rep. 2016; 6(33225); Alessio A P, Fili A E, Garrels W, Forcato D O, Olmos Nicotra M F, Liaudat A C, et al. Establishment of cell-based transposon-mediated transgenesis in cattle. Theriogenology. 2016; 85:1297-311. e2; Kim S, Saadeldin I M, Choi W J, Lee S J, Lee W W, Kim B H, et al. Production of transgenic bovine cloned embryos using piggybac transposition. J Vet Med Sci. 2011; 73:1453-7; Liu et al., Nature Communications volume 4, Article number: 2565 (2013); Sun et al., Scientific Reports volume 8, Article number: 15430 (2018); Luo et al., 2014. Efficient Generation of Myostatin (MSTN) Biallelic Mutations in Cattle Using Zinc Finger Nucleases. PLoS ONE 9(4): e95225, doi.org/10.1371/journal.pone.0095225; U.S. Pat. Pub 20110023158; Wang et al., Efficient TALEN-mediated gene knockin at the bovine Y chromosome and generation of a sex-reversal bovine. Cellular and Molecular Life Sciences volume 78, pages 5415-5425 (2021); Moghaddassi et al., (2014) TALEN-Mediated Modification of the Bovine Genome for Large-Scale Production of Human Serum Albumin. PLoS ONE 9(2): e89631, doi.org/10.1371/journal.pone.008963; and US Pat. Pub. 20170099813, which are all incorporated by reference herein as if expressed in their entireties and can be adapted for use with the present disclosure. These and other suitable genetic modifying systems for bovine genetic modification are described in greater below and in e.g., the Working Examples herein.

### 1. CRISPR-Cas Systems

In some embodiments, the donor nucleic acid (e.g., construct) is introduced (or knocked-in) into a recipient polynucleotide or genome using a CRISPR-Cas system, such as is shown in the Working Examples herein. In such embodiments, the site-directed nuclease is a Cas protein. In general, a CRISPR-Cas or CRISPR system as used in herein and in documents, such as WO 2014/093622, refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “guide RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008. CRISPR-Cas systems can be used to edit one or more nucleotides, remove one or more nucleotides, and/or delete one or more nucleotides.

Any suitable CRISPR-Cas system can be used in the context of the present disclosure to knock-in an engineered acrosome effector nucleic acid construct or polynucleotide into a genome of a cell. In some embodiments, the CRISPR-Cas system is a Class 2 system.

### a) Class 1 Systems

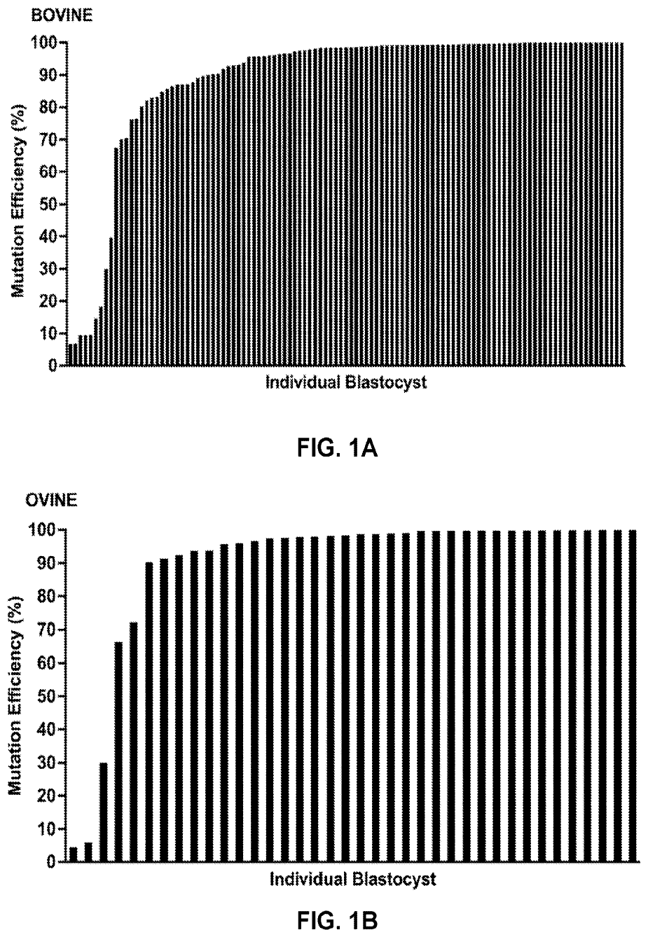

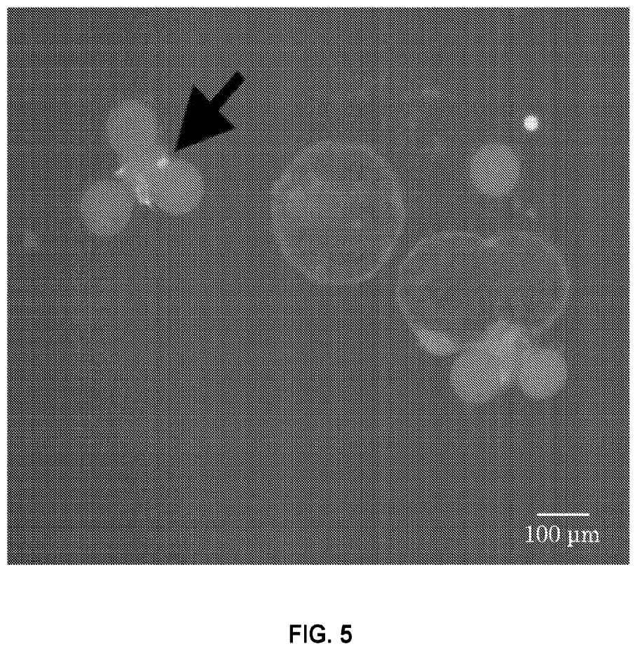

In some embodiments, the CRISPR-Cas system is a Class 1 CRISPR-Cas system. In certain example embodiments, the Class 1 system may be Type I, Type III or Type IV Cas proteins as described in Makarova et al. “Evolutionary classification of CRISPR-Cas systems: a burst of class 2 and derived variants” Nature Reviews Microbiology, 18:67-81 (February 2020), incorporated in its entirety herein by reference, and particularly as described in FIG. 1, p. 326; Koonin E V, Makarova K S. 2019 Origins and evolution of CRISPR-Cas systems. Phil. Trans. R. Soc. B 374: 20180087, DOI: 10.1098/rstb.2018.0087, particularly at FIGS. 1 and 2. In some embodiments, the Class 1 CRISPR-Cas system is a subtype Type I-A, I-B, I-C, I-U, I-D, I-E, and I-F, Type IV-A and IV-B, and Type III-A, III-D, III-C, and III-B system. In some embodiments, the Class 1 CRISPR-Cas system is a variant system, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al, the CRISPR Journal, v. 1, n5, FIG. 5.

### b) Class 2 Systems

In some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system is a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. In some embodiments, the CRISPR-Cas system is a Type II subtype, such as II-A, II-B, II-C1, or II-C2 system. In some embodiments, the Type II CRISPR-Cas system is a Cas9 system. In some embodiments, the CRISPR-Cas system is a Type V subtype, such as V-A, V-B1, V-B2, V-C, V-D, V-E, V-F1, V-F1(V-U3), V-F2, V-F3, V-G, V-H, V-I, V-K (V-U5), V-U1, V-U2, or V-U4 system. In some embodiments, the Type V CRISPR-Cas system includes a Cas12a (Cpf1), Cas12b (C2c1), Cas12c (C2c3), Cas12d (CasY), Cas12e (CasX), Cas14, and/or Cas(D. In some embodiments, the CRISPR-Cas system is a Type VI subtype, such as a VI-A, VI-B1, VI-B2, VI-C, or VI-D system. In some embodiments, the Type VI CRISPR-Cas system includes a Cas13a (C2c2), Cas13b (Group 29/30), Cas13c, and/or Cas13d.

### c) Guide RNAs

The CRISPR-Cas system described herein includes one or more guide RNAs (also referred interchangeably herein as “guide molecules” “guide polynucleotides” and “guide sequences”). The terms guide molecule, guide sequence and guide polynucleotide refer to polynucleotides capable of guiding Cas to a target genomic locus and are used interchangeably as in foregoing cited documents such as International Patent Publication No. WO 2014/093622. In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide. The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

The guide molecules can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. Target sequences are further discussed below.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments, the guide RNA (capable of guiding Cas to a target locus) can include (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and within the spirit and scope of this disclosure. Various modifications may be used to increase the specificity of binding to the target sequence and/or increase the activity of the Cas protein and/or reduce off-target effects. Example guide sequence modifications are described in International Patent Application WO2020033601, specifically paragraphs [0178]-[0333]. which is incorporated herein by reference.

### d) Ribonucleoprotein Complexes (RNPs)

In some embodiments, the site-directed nuclease is delivered into a cell as a ribonucleoprotein complex (RNP). As used herein, the term “ribonucleoprotein complex” or “RNP” and the like refers to a complex between a Cas protein, for example, Cas9 protein, and a crRNA (e.g., guide RNA or single guide RNA), a Cas protein and a trans-activating crRNA (tracrRNA), a Cas protein and a guide RNA, or a combination thereof (e.g., a complex containing a Cas protein, a tracrRNA, and a crRNA guide). The RNP method can often be used in cells that are difficult to transfect, such as primary cells. Using RNPs can also alleviate difficulties with protein expression that occur in cells where common eukaryotic promoters (such as CMV or EF1A promoters found in many CRISPR plasmids) are not expressed. Because this method does not require the delivery of foreign DNA, and the RNP is degraded over time, using RNPs may limit the potential for off-target effects. RNP delivery can also be useful for CRISPR applications where limited expression of Cas protein is required and specificity is a concern, such as knockout generation or homologous recombination.

The RNP can be assembled in vitro. One option is to purchase the Cas protein and a gRNA from a commercial vendor. Alternatively, the Cas protein can be expressed and purified using conventional cloning, cell culture, and protein expression/purification methods. gRNAs can be in vitro transcribed from ssDNA, which can be generated by commercial vendors such as IDT. These two components are then incubated together to form the RNP. Protocols for these steps are well-known and publicly available, e.g., www.protocols.io/groups/cornlab.

RNPs can be delivered to cells by a variety of methods, including physical approaches, materials-based delivery, responsive delivery systems, and targeted delivery systems such as described in Zhang S, et al. Strategies in the delivery of Cas9 ribonucleoprotein for CRISPR/Cas9 genome editing. Theranostic 11(2):614-648. 2021 Jan. 1, doi:10.7150/thno.47007. Physical approaches for RNP delivery include electroporation (as described elsewhere herein), microinjection, biolistics, microfluidics, filtroporation, nanotubes, induced transduction by osmocytosis and propanebetaine (iTOP). Materials based RNP delivery includes use of virus-like particles, lipid-nanoparticles (e.g. cell-derived extracellular vesicles, synthetic lipid nanoparticles), cell-penetrating peptides (CPPs), lipopeptides, polymers (e.g., dendrimers, PBAEs, PEGylated PLL, chitosan (CS) nanoparticles), nanogels, and inorganic nanoparticles (gold nanoparticles, metal-organic frameworks (MOFs), graphene oxide, black phosphorus nanosheets, calcium phosphate nanoparticles, DNA nanoclews). Responsive delivery systems for RNP delivery include light-responsive materials, ultrasound-responsive materials, reduction-sensitive materials, and pH-responsive materials. Targeted delivery systems for RNP delivery include galactose-based targeting, RGD (Arg-Gly-Asp) peptide-based targeting, other ligands or ligand-coated particles, and selective organ/tissue targeting.

In some embodiments, the site-directed nuclease is delivered to cells as an RNP using any of the above-described methods. In some instances, the RNP is delivered by electroporation.

In some instances, the RNPs can be delivered using nanoparticles (e.g., particles with a greatest dimension or greatest average dimension (e.g., diameter or greatest average diameter) no greater than 1000 nm). The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

In some instances, the RNPs can be delivered using virus-like particles, such as lentivirus-like particles. In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., a Cas protein-encoding polynucleotide and/or gRNA), and virus particle assembly, and secretion of mature virus particles into the culture media. Various other methods and techniques are generally known to those of ordinary skill in the art. Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ or more particles/mL.

### e) Target Sequences, PAMs, and PFSs

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or including the target sequence. Likewise, a “target polynucleotide” as used in this context herein refers to a polynucleotide sequence being or including the target sequence for a guide polynucleotide. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide can be DNA. The target polynucleotide can be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### f) PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 1 (from Gleditzsch et al. 2019) below shows several Cas polypeptides and the PAM sequence they recognize.

TABLE 1
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) | TTTV |
| (including LbCpf1 | |
| and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: dx.doi.org/10.1101/091611 (Dec. 4, 2016). Doench et al. 2014 Nat Biotechnol. 2014 December; 32(12):1262-7 created a pool of sgRNAs, tiling across all possible target sites of a panel of six endogenous mouse and three endogenous human genes and quantitatively assessed their ability to produce null alleles of their target gene by antibody staining and flow cytometry. Doench et al. can demonstrate that optimization of the PAM improved activity and also provided an on-line tool for designing sgRNAs. Such approaches can be adapted for use with the present disclosure.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016.Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### g) Nuclear Targeting and Transportation Sequences

For modification of nuclear located polynucleotides including, but not limited to, genomic DNA one or more components of the CRISPR-Cas system can include one or more sequences or signals for nucleus targeting and/or transportation. Although these are discussed with specific reference to CRISPR-Cas systems, such sequences and signals can be applied to other genetic modification systems or components thereof discussed elsewhere herein.

Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 6) or PKKKRKVEAS (SEQ ID NO: 7); the NLS from nucleoplasmin (e.g., the nucleoplasmin bipartite NLS with the sequence KRPAATKKAGQAKKKK (SEQ ID NO: 8)); the c-myc NLS having the amino acid sequence PAAKRVKLD (SEQ ID NO: 9) or RQRRNELKRSP (SEQ ID NO: 10); the hRNPA1 M9 NLS having the sequence NQSSNFGPMKGGNFGGRSSGPYGGGGQYFAKPRNQGGY (SEQ ID NO: 11); the sequence RMRIZFKNKGKDTAELRRRRVEVSVELRKAKKDEQILKRRNV (SEQ ID NO: 12) of the IBB domain from importin-alpha; the sequences VSRKRPRP (SEQ ID NO: 13) and PPKKARED (SEQ ID NO: 14) of the myoma T protein; the sequence PQPKKKPL (SEQ ID NO: 15) of human p53; the sequence SALIKKKKKMAP (SEQ ID NO: 16) of mouse c-abl IV; the sequences DRLRR (SEQ ID NO: 17) and PKQKKRK (SEQ ID NO: 18) of the influenza virus NS1; the sequence RKLKKKIKKL (SEQ ID NO: 19) of the Hepatitis virus delta antigen; the sequence REKKKFLKRR (SEQ ID NO: 20) of the mouse Mx1 protein; the sequence KRKGDEVDGVDEVAKKKSKK (SEQ ID NO: 21) of the human poly(ADP-ribose) polymerase; and the sequence RKCLQAGMNLEARKTKK (SEQ ID NO: 22) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In CRISPR-Cas systems including a deaminase, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In some embodiments, a component of the CRISPR-Cas system includes a one or more nuclear export signals (NES), one or more one or more nuclear localization signals (NLS), or any combinations thereof. In some cases, the NES may be an HIV Rev NES. In certain cases, the NES may be MAPK NES. When the component is a protein, the NES or NLS may be at the C terminus of component. In some embodiments, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### h) Donor Templates

In some embodiments the CRISPR-Cas system includes a donor nucleic acid such as a donor template, e.g., a recombination template, as discussed elsewhere in this disclosure. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp.

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some embodiments, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149). The strategy and techniques Of Suzuki et al. can be adapted for use with the present disclosure.

### 2. Specialized Cas-Based Systems

i. Dead Cas (dCas) Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoDI, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (International Patent Publication Nos. WO2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

ii. Split-Cas Systems

In some embodiments, the CRISPR-Cas system is a split CRISPR-Cas system. See e.g., Zetche et al., 2015. Nat. Biotechnol. 33(2): 139-142 and International Patent Publication WO2019/018423, the compositions and techniques of which can be used in and/or adapted for use with the present disclosure. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wildtype Cas allows other methods of delivery of the systems to the cells, such as the use of cell penetrating peptides as described herein.

iii. DNA and RNA Base Editing Systems

In some embodiments, a genomic edit is made using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018.Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present disclosure described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO2018/213708, WO2018/213726, WO2019126709, WO2019126716, and WO2019126762, each of which is incorporated herein by reference and can be adapted for use with the present disclosure.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO2019/005886, and WO2019/071048, WO2019126709, which are incorporated herein by reference and can be adapted for use with the present disclosure. An example FnCas9 system that may be adapted for RNA base editing purposes is described in International Patent Publication No. WO2016/106236, which is incorporated herein by reference and can be adapted for use with the present disclosure.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference and can be adapted for use with the present disclosure.

iv. Prime Editor Systems

In some embodiments, a genomic edit is made using a prime editing system. See e.g., Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present disclosure include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

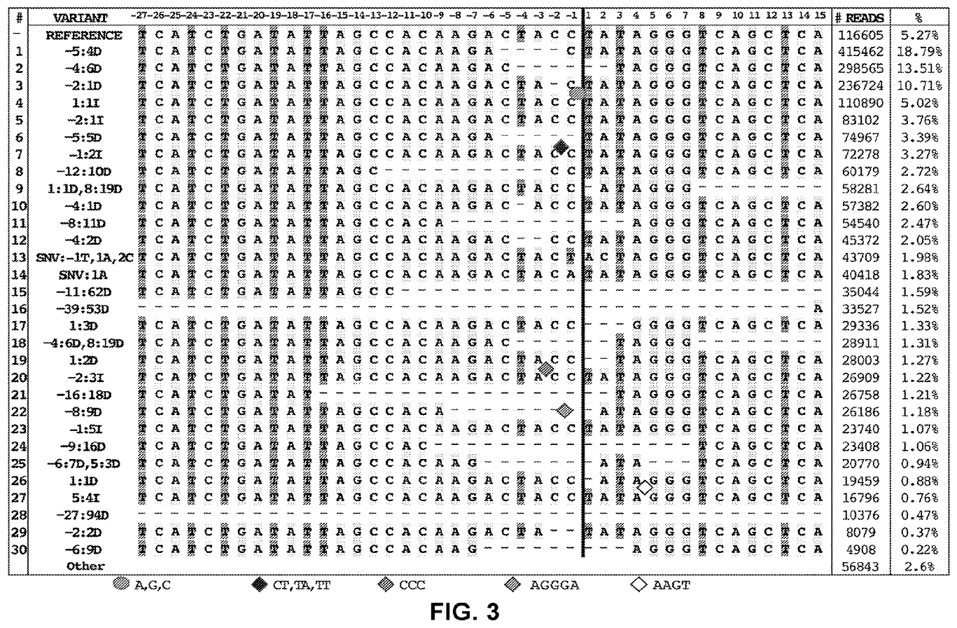

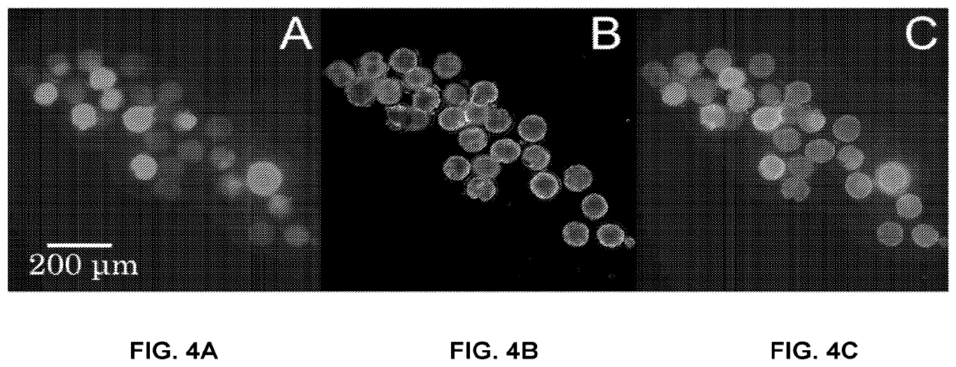

In some embodiments, the prime editing system can be a PE1 system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4.

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

v. CRISPR Associated Transposase (CAST) Systems

In some embodiments, a genomic edit can be made using a CRISPR Associated Transposase (“CAST”) system. The CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class 1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference and can be adapted for use with the present disclosure.

### 3. TALE Nucleases

In some instances, the site-directed nuclease is a TALE polypeptide. In some embodiments, a TALE nuclease or TALE nuclease system can be used to knock-in an engineered nucleic acid construct or polynucleotide of interest into a genome of a cell. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or X₃₄ or X₃₅, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or X₃₄ or X₃₅)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods and other aspects of the present disclosure can be isolated, non-naturally occurring, recombinant or engineered nucleic acid-binding proteins that have nucleic acid or DNA binding regions containing polypeptide monomer repeats that are designed to target specific nucleic acid sequences.

Polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the present disclosure will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the present disclosure may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

In some embodiments, the TALEs can include N- and/or C-terminal capping regions, which can increase TALE polypeptide binding efficiency (see e.g., Zhang et al., Nature Biotechnology 29:149-153 (2011). Such “capping regions” can be directly N-terminal and/or C-terminal of the DNA binding region of a TALE. Exemplary amino acid sequence of a N-terminal capping region and C-terminal capping regions are generally known in the art.

As used herein, the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides described herein.

In some embodiments, the entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain an N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. In some embodiments, the C-terminal capping region includes only or at least the 68 C-terminal amino acids, which enhance binding activity equal to the full-length capping region. See e.g., Zhang et al., Nature Biotechnology 29:149-153 (2011). In some embodiments, the C-terminal capping region includes only or at least the 20 C-terminal amino acids, which have about 50% or greater the efficacy of the full-length capping region. See e.g., Zhang et al., Nature Biotechnology 29:149-153 (2011).

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the present disclosure may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Krüppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the present disclosure may include any combination of the activities described herein.

A variety of additional TALEN-based systems have been described in the art, and modifications thereof are regularly reported; see, e.g., Boch, Science 326(5959):1509-12 (2009); Mak et al., Science 335(6069):716-9 (2012); and Moscou et al., Science 326(5959):1501 (2009). The use of TALENs based on the “Golden Gate” platform, or cloning scheme, has been described by multiple groups; see, e.g., Cermak et al., Nucleic Acids Res. 39(12):e82 (2011); Li et al., Nucleic Acids Res. 39(14):6315-25 (2011); Weber et al., PLoS One. 6(2):e16765 (2011); Wang et al., J Genet Genomics 41(6):339-47, Epub 2014 May 17 (2014); and Cermak T et al., Methods Mol Biol. 1239:133-59 (2015), any of which can be adapted for use with the present disclosure.

### 4. Zinc Finger Nucleases

In some embodiments, the site-directed nuclease is a zinc finger protein. In some embodiments, a zinc finger system is used to knock-in or otherwise introduce an engineered nucleic acid construct or polynucleotide of interest into a genome of a cell. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

Zinc Finger proteins can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. These and any other ZFN systems can be used to knock-in an engineered acrosome effector nucleic acid construct or polynucleotide as described herein into a genome of a cell. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference and whose systems and methods can be adapted for use with the present disclosure to generate an engineered acrosome effector expressing cell and/or organism.

A variety of ZFN-based systems have been described in the art, modifications thereof are regularly reported, and numerous references describe rules and parameters that are used to guide the design of ZFNs; see, e.g., Segal et al., Proc Natl Acad Sci USA 96(6):2758-63 (1999); Dreier B et al., J Mol Biol. 303(4):489-502 (2000); Liu Q et al., J Biol Chem. 277(6):3850-6 (2002); Dreier et al., J Biol Chem 280(42):35588-97 (2005); and Dreier et al., J Biol Chem. 276(31):29466-78 (2001).

### 5. Homing Endonucleases

In some embodiments, the genetic modifying system, or site-directed nuclease, is or includes one or more homing endonucleases. Homing endonucleases (HEs) are sequence-specific endonucleases that have long recognition sequences (14-44 base pairs) and cleave DNA with high specificity-often at sites unique in the genome. There are at least six known families of HEs as classified by their structure, including GIY-YIG, His-Cis box, H—N—H, PD-(D/E)xK, and Vsr-like that are derived from a broad range of hosts, including eukaryotes, protists, bacteria, archaea, cyanobacteria and phage. As with ZFNs and TALENs, HEs can be used to create a DSB at a target locus as the initial step in genome editing. In addition, some natural and engineered HEs cut only a single strand of DNA, thereby functioning as site-specific nickases. The large target sequence of HEs and the specificity that they offer have made them attractive candidates to create site-specific DSBs.

A variety of HE-based systems have been described in the art, and modifications thereof are regularly reported; see, e.g., the reviews by Steentoft et al., Glycobiology 24(8):663-80 (2014); Belfort and Bonocora, Methods Mol Biol. 1123:1-26 (2014); Hafez and Hausner, Genome 55(8):553-69 (2012); and references cited therein, which can be adapted for use with the present disclosure.

### 6. Meganucleases and Hybrid Meganucleases

In some embodiments, the site-directed nuclease is a meganuclease or a hybrid mega nuclease. In some embodiments, a meganuclease, a hybrid mega nuclease, or system thereof can be used to introduce a polynucleotide, such as an engineered nucleic acid construct or polynucleotide of interest, into a genome of a cell. Meganucleases are endodeoxyribonucleases that are characterized by a large recognition site (double-stranded DNA sequences of 12 to 40 base pairs). Exemplary meganucelases and methods for using meganucleases can be found in U.S. Pat. Nos. 8,163,514, 8,133,697, 8,021,867, 8,119,361, 8,119,381, 8,124,369, and 8,129,134, which are specifically incorporated herein by reference. Such methods can be adapted for use to generate a cell and/or organism capable of expressing an engineered acrosome effector polynucleotide and/or polypeptide of the present disclosure.

Exemplary hybrid meganucleases include, without limitation, the MegaTal system and Tev-mTALEN systems, which use a fusion of TALE DNA binding domains and catalytically active HEs, taking advantage of both the tunable DNA binding and specificity of the TALE, as well as the cleavage sequence specificity of the HE; see, e.g., Boissel et al., NAR 42: 2591-2601 (2014); Kleinstiver et al., G3 4:1155-65 (2014); and Boissel and Scharenberg, Methods Mol. Biol. 1239: 171-96 (2015). Other exemplary hybrid meganucleases include, without limitation, the MegaTev system, which includes fusion of a meganuclease (Mega) with the nuclease domain derived from the GIY-YIG homing endonuclease I-TevI (Tev) where two active sites are positioned about 30 bp apart on a DNA substrate and generate two DSBs with non-compatible cohesive ends; see, e.g., Wolfs et al., NAR 42, 8816-29 (2014).

### 7. Transposon Systems

In some embodiments, a transposon system can be used to knock-in an engineered acrosome effector nucleic acid construct or polynucleotide as described herein into a genome of a cell. Exemplary transposons systems that can be utilized for modifying a polynucleotide are described herein and will be appreciated by those of ordinary skill in the art in view of this disclosure. In some embodiments, the transposon system is a Class I transposon system polypeptide. In some embodiments, the transposon system is a Class II transposon system polypeptide. As used herein, “transposon” (also referred to as transposable element) refers to a polynucleotide sequence that is capable of moving from one location in a genome to another location. There are several classes of transposons. Transposons include retrotransposons (Class I transposons) and DNA transposons (Class II transposons). Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide.

Suitable Class I transposon systems include any of those in, without limitation, LTR and non-LTR retrotransposon systems. Exemplary Class I transposon systems include, without limitation, CRE, R2, R4, L1, RTE, Tad, R1, LOA, I, Jockey, CR1 polypeptides. See e.g., Proc Natl Acad Sci USA. 2006 Nov. 21; 103(47):17602-7; Eickbush T H et al, Integration, Regulation, and Long-Term Stability of R2 Retrotransposons, Microbiol Spectr. 2015 April; 3(2):MDNA3-0011-2014. doi: 10.1128/microbiolspec.MDNA3-0011-2014; Han J S, Non-long terminal repeat (non-LTR) retrotransposons: mechanisms, recent developments, and unanswered questions, Mob DNA. 2010 May 12; 1(1):15. doi: 10.1186/1759-8753-1-15; Malik H S et al., The age and evolution of non-LTR retrotransposable elements, Mol Biol Evol. 1999 June; 16(6):793-805, which are incorporated by reference herein in their entireties.

Suitable Class II transposon systems include any of those in, without limitation, the following transposon systems: Sleeping Beauty transposon system (Tcl/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tcl/mariner superfamily) (see e.g., Miskey et al. 2003 Nucleic Acid Res. 31(23):6873-6881) and variants thereof. In some embodiments, the Class II transposon system is a DD[E/D]transposon or transposon polypeptide. In some embodiments, the Class II transposon psystem is a Tcl/mariner, PiggyBac, Frog Prince, Tn3, Tn5, hAT, CACTA, P, Mutator, PIF/Harbinger, Transib, or a Merlin/IS1016 transposon polypeptide.

Suitable Class II transposon systems and components that can be utilized in the context of the present disclosure include and are not limited to those described in e.g., Han et al., 2013. BMC Genomics. 14:71, doi: 10.1186/1471-2164-14-71, Lopez and Garcia-Perez. 2010. Curr. Genomics. 11(2):115-128; Wessler. 2006. PNAS. 103(47): 176000-17601; Gao et al., 2017. Marine Genomics. 34:67-77; Bradic et al. 2014. Mobile DNA. 5(12) doi:10.1186/1759-8753-5-12; Li et al., 2013. PNAS. 110(25)E2279-E2287; Kebriaei et al. 2017. Trends in Genetics. 33(11): 852-870); Miskey et al. 2003. Nucleic Acid res. 31(23):6873-6881; Nicolas et al. 2015. Microbiol Spectr. 3(4) doi: 10.1128/microbiolspec.MDNA3-0060-2014); W. S. Reznikoff. 1993. Annu Rev. Microbiol. 47:945-963; Rubin et al. 2001. Genetics. 158(3): 949-957; Wicker et al. 2003. Plant Physiol. 132(1): 52-63; Majumdar and Rio. 2015. Microbiol. Spectr. 3(2) doi: 10.1128/microbiolspec.MDNA3-0004-2014; D. Lisch. 2002. Trends in Plant Sci. 7(11): 498-504; Sinzelle et al. 2007. PNAS. 105(12): 4715-4720; Han et al. 2014; Genome Biol. Evol. 6(7):1748-1757; Grzebelus et al. 2006; Mol. Genet. Genomics. 275(5):450-459; Zhang et al. 2004. Genetics. 166(2):971-986; Chen and Li. 2008. Gene. 408(1-2):51-63; and C. Feschotte. 2004. Mol. Biol. Evol. 21(9):1769-1780.

### 8. Recombinase Systems

In some embodiments, the genetic modification system to modify a genome, such as knock-in a transgene, or knock out an endogenous gene, is a recombinase system. Generally, recombinases are enzymes that catalyze site-specific recombination events, and recombination systems employ such enzymes to achieve site-specific polynucleotide integration or disruption. Many recombinase systems for gene knock-in, gene knock-out, and other genome or polynucleotide are generally known in the art since their introduction several decades ago (see e.g., Sauer, B. Mol Cell Biol 7(6):2087-2096 (1987)) and can be used in the context of the present disclosure to introduce a transgene of the present disclosure and/or one or more components of another genetic modifying system described herein and/or generally known to a genome of a cell or another polynucleotide. Exemplary systems include without limitations, Cre-lox and FLP-FRT systems (see e.g., Maizels et al., J. Immunol. 2013. 161(1): doi:10.4049/jimmunol.1301241; Graham et al., Biotech J. 2009. 4(1):108-118; Chen et al. Animal. 4(5):767-771 (2010); Kalds et al. Front. Genet. 2019, doi.org/10.3389/fgene.2019.00750; Gurusinghe et al., J Cell Biochem. 2017. 118(5):1201-1215; and Wang et al., Plant Cell Rep (2011) 30:267-285), which are each incorporated by reference as if expressed in their entirety and can be adapted for use with the present disclosure.

### IV. Engineered Cells and Organisms

Described herein are engineered cells and organisms, particularly non-human animals, that contain and/or express engineered nucleic acids, vector(s), and/or polypeptides that are generated using the methods of the present disclosure. Also described herein are bodily fluids that can contain one or more engineered cells, such as oocytes and semen. In some embodiments, the bodily fluid containing the engineered cell(s) is be produced by an engineered organism, such as a non-human animal, particularly a bovine. In some embodiments, the engineered organism produces gametes containing a desired chromosome and/or allele (i.e., a non-targeted chromosome or allele).

In certain example embodiments a cell, population thereof, or progeny thereof contain and/or expresses an engineered nucleic acid construct or encoding polynucleotide as described elsewhere herein, a vector or vector system as described elsewhere herein, or any combination thereof. In certain example embodiments, the cell is a gamete. In certain example embodiments, the cell is a spermatid or a spermatozoa. In certain example embodiments, the cell is an oocyte or an ovum. In certain example embodiments, the cell is diploid or haploid.

In certain example embodiments, the cell is eukaryotic or prokaryotic. Prokaryotic cells are useful, for e.g., vector or nucleic acid amplification or propagation as is discussed elsewhere herein. In certain example embodiments, the cell is a non-human mammalian or avian cell. In certain example embodiments, the cell is a bovine cell, an equine cell, a porcine cell, an ovine cell, a caprine cell, a camelid cell, a cervine cell, a canine cell, a feline cell, a murine cell, a leporine cell, or a cavine cell. In certain example embodiments, the nucleic acid construct is integrated in or otherwise associated with one or more target chromosomes and/or alleles.

In some embodiments, the engineered cell is a donor cell that can be introduced into a non-human animal. In some embodiments, the engineered cell is a self-renewing cell or totipotent cell. In some embodiments, the non-human animal is a bovine, an equine, a porcine, an ovine, a caprine, a camelid, a cervine, a canine, a feline, a murine, a leporine, or a cavine.

In certain example embodiments, the target chromosome is the Y or one or both of the X chromosomes. In certain example embodiments, the one or more target alleles are pathogenic or undesirable alleles. In certain embodiments, the target alleles contain a genetic abnormality, such as a genetic abnormality that leads to a genetic disease or disorder. In some embodiments, the genetic disease or disorder is Alpha (a) and/or Beta (ß)-Mannosidosis, Arthrogryposis Multiplex (AM), Contractural Arachnodactyly (CA), Neuropathic Hydrocephalus (NH), Hypotrichosis (hairless calf), Idiopathic Epilepsy, Osteopetrosis, Protoporphyria, Pulmonary Hypoplasia and Anasarca (PHA), Tibial Hemimelia (TH), achondroplasia (bulldog dwarfism), alopecia, ankylosis, arthrogryposis (palate-pastern syndrome, rigid joints), brachynathia inferior (parrot mouth), cryptorchidism, dermoid, double muscling, fawn calf syndrome, hypotrichosisi (rat tail), neuraxial edema (maple syrup urine disease), oculocutaneous hypopigmentation, polydactyly, progressive bovine myeloencephalym prolonged gestation, syndactyly (mule foot), translocations, Bovine leukocyte adhesion deficiency, Complex Vertebral Malformation, freemartinism, or any combination thereof. In some embodiments, the disease or disorder is any one described in Ciploch et al., Genes. Genomics. 2017 39(5):461-471. In some embodiments, the allele(s) that are targeted contain one or more genes set forth in Cieploch et al., Genes & Genomics Vol. 39, pages 461-471 (2017), particularly at Table 1.

In certain example embodiments, the non-human organism is a mammal or an avian. In certain example embodiments, the non-human organism is a bovine, an equine, a porcine, an ovine, a caprine, a camelid, a cervine, a canine, a feline, a murine, a leporine, or a cavine. In certain example embodiments, the non-human organism is a male. In certain example embodiments, the non-human organism is a female.

The engineered organisms containing or expressing engineered nucleic acids, vector(s), and/or polypeptides as described herein can be developed using one or more suitable techniques for generating transgenic non-human animals such as those described elsewhere herein and generally known in the art, including but not limited to somatic cell nuclear transfer, oocyte pronuclear DNA microinjection, zygote microinjection, or embryo microinjection, intracytoplasmic sperm injection, in vitro fertilization, embryo transfer, in vitro embryo culture, or any combination thereof.

Progeny of the engineered organism described herein can be obtained by any suitable method or technique including natural mating, in vitro fertilization, artificial insemination, embryo transfer, and/or the like.

Also provided are methods of generating non-human animals by mating an engineered non-human animal described herein with a non-engineered non-human animal or another engineered non-human animal described herein. In some embodiments, at least one of the engineered non-human animals mated is male. In some embodiments, at least one of the engineered non-human animals mated is female. In some embodiments, an engineered non-human male animal is mated to a non-engineered non-human female animal. In some embodiments, an engineered non-human male animal is mated to an engineered non-human female animal. In some embodiments, an engineered non-human female animal is mated to a non-engineered non-human male animal. Mating can be by any suitable technique including, but not limited to, any suitable method or technique including natural mating, in vitro fertilization, artificial insemination, embryo transfer, and/or the like. In some embodiments, mating involves artificial insemination using semen from an engineered non-human animal of the present disclosure described herein.

Also described in several example embodiments herein are methods of cell selection, detection, and/or identification comprising using the engineered nucleic acid constructs and/or engineered polypeptides described herein, particularly those that do or do not comprise a target chromosome and/or allele. In certain example embodiments, the cells that comprise the target chromosome and/or allele comprise, and optionally express, an engineered nucleic acid, a vector or vector system, or any combination thereof. In some embodiments, the method of cell selection can further include sorting and/or separating cells that comprise the target chromosome and/or allele from cells that do not. In some embodiments, sorting and/or separating cells that comprise the target chromosome and/or allele from cells that do not include separation or sorting based on morphology, expression of a reporter, functionality, activity, or other measurable or detectable phenotype. In some embodiments, sorting and/or separating cells comprises microscopy, fluorescence activated cell sorting, density gradient centrifugation, immunodensity cell isolation, immunomagnetic cell separation, microfluidic cell sorting, buoyancy-activated cell sorting, and others generally used in the art.

Further embodiments are illustrated in the following Examples which are given for illustrative purposes only and are not intended to limit the scope of the present disclosure or combination of features of the claims according to the present disclosure.

### EXAMPLES

### I. Example 1—Optimization of Electroporation and Adeno-Associated Virus-Mediated Generation of 2.7 KB Knock-In Livestock Blastocysts

### A. Introduction

Genetic improvement of livestock species is critical for the efficient production of animal-source foods. However, traditional breeding approaches to introduce useful genetic variation through crossbreeding are both slow and often results in linkage drag (Ref 1). Gene-editing technologies offer an approach to rapidly introduce targeted genetic changes in livestock genomes to augment traditional selective breeding approaches. Previously, editing in livestock has mostly been achieved through somatic cell nuclear transfer of an edited cell. This constrains editing to the genetic diversity available in cell lines, and cloning artifacts are common (Ref. 2).

Conversely, introducing gene-editing reagents into developing mammalian zygotes allows modification of the next generation and has typically been achieved using cytoplasmic microinjection. This time-consuming procedure requires expensive equipment and a high level of technical skill, rendering it unscalable and inaccessible for laboratories without specialized equipment or personnel (Ref. 1). In addition, microinjection often results in genetic mosaicism, the presence of more than one genotype within an individual. This does not pose an insurmountable issue for mouse production as it can be quickly bred out, however, livestock species have longer generation intervals meaning mosaicism cannot be quickly nor inexpensively bred out.

Electroporation is a widely used technique in biotechnology and medicine for delivering drugs and genes into living cells and it can be employed as a quick and simple approach to introducing gene-editing reagents into zygotes (Ref. 3). Electroporation has been used to introduce gene-editing reagents into early-stage livestock embryos including porcine (Refs 0.4-14), bovine (Refs. 12,15-18) and ovine and caprine embryos (Ref. 19). There are currently no articles reporting large (>1 kb) targeted insertions in mammalian livestock embryos using electroporation alone. This may be in part due to the presence of the zona pellucida (ZP), a hard glycoprotein matrix surrounding zygotes that has been shown to impede the movement of large nucleic acid fragments into embryos (Ref. 20).

This complicates the production of gene-edited animals harboring useful exogenous genes as homology-directed repair (HDR) templates containing a gene and promoter often result in DNA cassettes that are >1 kb. Previous efforts to produce mammalian embryos harboring targeted insertions >1 kb have required the removal of either the ZP or microinjection of donor template before electroporation (Refs. 7, 18, 21). Removal of the ZP before electroporation requires a strict protocol that results in sticky and damaged embryos that become difficult to work with, whereas microinjection of the donor template before electroporation defeats the purpose of using electroporation as a scalable and high-throughput approach to generating gene-edited animals.

Adeno-associated viruses (AAVs; recombinant adeno-associated virus [rAAV]) have been employed to deliver nucleic acids to various cell types for many years. They are favored for their nonpathogenic nature, ability to package either single-stranded or self-complementary DNA, and 4.9 kb capacity to efficiently transduce mammalian cells (Refs. 22-24). The genome of wildtype AAVs contains only four genes (rep, cap, aap, maap) flanked by inverted terminal repeats (ITRs) on both sides. The rep gene is required for viral genome replication and packaging, the cap gene produces viral capsids, the aap gene promotes capsid assembly, and the maap gene helps facilitate viral replication (Refs. 24, 25).

Conversely, rAAV does not contain these genes and only requires the presence of 130 bp AAV ITR arms flanking a DNA fragment of up to 4.9 kb on either side for packaging (Ref. 26). The ITRs are the only cis-acting components necessary for the packaging and replication of DNA fragments (Ref. 27). AAV vectors lack an integrase activity and are considered as nonintegrating. These qualities make rAAV an ideal vector to transduce embryos to deliver HDR templates for producing targeted knock-ins (KIs).

rAAV has been used to successfully transduce DNA fragments into fertilized rat and mouse zygotes in the absence of ZP treatment before electroporation (Refs. 20, 22, 28, 29). The protocols for utilizing rAAV and electroporation to generate KI embryos have proven to be high throughput and easy to use, however, such methods have not been utilized in livestock species. To date, the largest donor cassette used to produce a targeted insertion with rAAV and electroporation was a 4.3 kb template in mice (Ref. 22). There are currently no reports of the production of KI mammalian livestock embryos using electroporation and rAAV.

A first aim of the present example was to optimize electroporation parameters for the efficient targeted mutation of the H11 safe harbor locus in bovine and ovine zygotes. Based on previous literature, a range of parameters including poring pulse voltage, number, duration, and polarity were tested to electroporate clustered regularly interspaced palindromic repeat (CRISPR)/CRISPR-associated protein 9 (Cas9) and single guide RNA (sgRNA) reagents into zygotes and oocytes targeting the H11 locus. This locus was selected due to its ability to incorporate and express exogenous DNA fragments, and disrupting its sequence does not pose any known risks to the host organism's health.

This makes it a suitable candidate for potential future KI studies (Refs. 30,31). Electroporated zygotes and oocytes that developed to the blastocyst stage were collected and analyzed for mutation rates. The optimal electroporation settings were then used to target an alternative locus in bovine and ovine (the Rosa26 locus and the bone morphogenetic protein receptor type II [Bmpr2] gene, respectively) to confirm mutation and development efficiency. In addition, a subset of embryos electroporated with these settings targeting the H11 locus underwent next-generation sequencing (NGS) to identify levels of genetic mosaicism, and the most frequent DNA break repair outcomes.

A second aim of the present example was to generate bovine blastocysts harboring a 2.7 kb green fluorescent protein (GFP) reporter gene KI at the H11 locus using electroporation and rAAV infection. First, a panel of six natural rAAV serotypes (1, 2, 5, 6, 8, and 9) packaged with a human cytomegalovirus (CMV) promoter-enhanced green fluorescent protein (eGFP; CMV-eGFP) reporter were tested at various concentrations for transduction efficiency into early bovine embryos. Then a 3.9 kb HDR template was packaged into the most efficient rAAV serotype and incubated with bovine oocytes and sperm for 6 h before electroporation of the Cas9:sgRNA ribonucleoprotein (RNP) to produce bovine blastocysts harboring a 2.7 kb targeted insertion at the H11 locus.

### B. Results

### 1. Bovine Optimization

Various electroporation parameters were tested on bovine zygotes for the delivery of Cas9:sgRNA RNP to induce targeted mutations at the H11 locus. The blastocyst development and mutation rates of treated bovine blastocysts were then evaluated for the different electroporation conditions (Table 2). First, five 1 ms bipolar poring pulses at 15 and 20V/mm were trialed based on previous publications. A significant reduction in embryo development was observed when comparing 20V/mm with 15V/mm, but the latter was associated with low rates of mutation (7%) as observed with Tracking of Indels by DEcomposition (TIDE) analysis.

TABLE 2
The electroporation parameters used to introduce CRISPR-associated protein 9:single guide RNA
ribonucleoprotein into bovine oocytes, and their development and mutation rates

| | | | | | | | | | | | | treated/ |
|---|---|---|---|---|---|---|---|---|---|---|---|---|
| | | | | | | | | | | | Avg. | control |
| | | | | | | | | Blasto- | | | Control | blasto- |
| | | | No | Pulse | | De- | | cysts | | | blasto- | cyst |
| | | Pulse | of | Inter- | Po- | cay | Em- | Formed, | Blasto- | Muta- | cyst | develop- |
| | Volts. | length | Pul- | val | lar- | rate | bryos | % | cysts | tion, | rate, % | ment |
| Group | V/mm | (ms) | ses | (ms) | ity | (%) | Treated | (n) | analyzed | % (n) | (n) | ratio |
| A | 20 | 3 | 2 | 50 | Uni | 0 | 246 | 47 (115) | 80 | 79 (71)ª | 48 (150) | 0.98ª |
| B | 20 | 3 | 2 | 50 | Bi | 0 | 703 | 27 (191) | 84 | 96 (81)^{b} | 43 (300) | 0.63^{b} |
| C | 20 | 3 | 3 | 50 | Uni | 0 | 55 | 25 (14) | 14 | 50 (7)^{c} | 28 (50) | 0.89^{c} |
| D | 20 | 3 | 3 | 50 | Bi | 0 | 270 | 31 (83) | 74 | 93 (69)^{b} | 43 (150) | 0.72ª |
| E | 20 | 1 | 5 | 50 | Bi | 0 | 58 | 5 (3) | 0 | — | 28 (50) | 0.18^{e} |
| F | 15 | 1 | 5 | 50 | Bi | 0 | 154 | 23 (35) | 15 | 7 (1)^{d} | 38 (50) | 0.61^{b} |

Values in the same column with same superscript letters do not differ significantly (p < 0.05).

The number of pulses was then decreased while the length of the pulse was increased to 3 ms at 20 V/mm, and comparisons were made between two pulses to three pulses, and unipolar to bipolar pulses. A unipolar pulse resulted in a significantly higher blastocyst development rate (p<0.05) as compared with a bipolar pulse. However, the opposite was noted in the mutation rate. Of note, mutation AU8c rates of 96% and 93% (p=0.30, NS) were achieved when using two and three bipolar pulses, respectively.

The electroporation conditions of 20 V/mm, and three bipolar 3 ms pulses were then used to electroporate bovine zygotes with Cas9:sgRNA RNP targeting the Rosa26 locus. Similar results were obtained with a mutation rate of 84% (n=19), and a blastocyst development rate of 12% (n=146; control 25%, n=20) (data not shown).

### 2. Ovine Optimization

Various electroporation parameters were also tested on ovine zygotes and activated oocytes for the delivery of Cas9:sgRNA RNP to induce targeted mutations at the H11 locus (Table 3). First, four unipolar pulses at 40 V/mm, followed by five bipolar 5 V/mm transfer pulses were trialed based on previous experiments at UC Davis (Ref 19). However, this resulted in a significant reduction in blastocyst formation rate (1.8%; p<0.05). The number of pulses was, therefore, decreased to two 3.5 ms unipolar pulses at 40 V/mm. This improved the blastocyst formation rate to 30% (n=25; control 60%, n=25), but the mutation rate was only 74%.

TABLE 3
The electroporation parameters used to introduce CRISPR-associated protein 9:single
guide RNA ribonucleoprotein used for ovine zygotes and activated oocytes, and their
development and mutation rates

| | | | | | | | | | | | | Trea- |
|---|---|---|---|---|---|---|---|---|---|---|---|---|
| | | | | | | | | | | | | ted/ |
| | | | | | | | | | | | | Con- |
| | | | | | | | | | | | | trol |
| | | | | | | | | | | | Avg. | blasto- |
| | | | | | | | | Blasto- | | | Control | cyst |
| | | | No. | Pulse | | De- | | cysts | | | blasto- | de- |
| | | Pulse | of | Inter- | Po- | cay | Em- | Formed, | Blasto- | Muta- | cyst | velop- |
| | Volts. | length | Pul- | val | lar- | rate | bryos | % | cysts | tion, | rate, % | ment |
| Group | V/mm | (ms) | ses | (ms) | ity | (%) | Treated | (n) | analyzed | % (n) | (n) | ratio |
| A | 40 | 3.5 | 2 | 50 | Uni | 0 | 83 | 30 (25) | 18 | 72 (13)^{ac} | 60 (25) | 0.50ª |
| B | 40 | 3.5 | 2 | 50 | Bi | 0 | 364 | 18 (64) | 45 | 89 (40)^{ab} | 46 (130) | 0.39^{ab} |
| C | 30 | 3.5 | 2 | 50 | Bi | 0 | 537 | 14 (76) | 37 | 97 (36)^{b} | 38 (175) | 0.37^{b} |
| D | 30 | 3.5 | 3 | 50 | Bi | 0 | 507 | 6 (30) | 21 | 95 (20)^{b} | 30 (245) | 0.20^{c} |
| E | 40 | 3.5 | 3 | 50 | Bi | 0 | 163 | 9 (14) | 7 | 57 (4)^{c} | 26 (70) | 0.35^{b} |
| F | 40, | 1.5, | 4, | 50 | Uni, | 10, | 220 | 2 (14) | 0 | — | 39 (125) | 0.05^{d} |
| | 5 | 50 | 5 | | Bi | 40 | | | | | | |

If a transfer pulse was also included, the parameters are noted after a comma.
Values in the same column with same superscript letters do not differ significantly (p < 0.05).

Two 3.5 ms 40 V/mm bipolar pulses increased the mutation rate to 89% (n=45), but reduced development to 18% (n=64, control 46%, n=130). These same parameters at 30 V/mm resulted in a slightly lower, although not statistically significant, blastocyst development rate. It should be noted that the untreated control development rates were lower for this set of 30 V/mm bipolar experiments, and although development rates are expressed as a proportion of controls, this can be a confounding factor in experiments when oocyte collection occurs at different times of the year with seasonal breeders like sheep.

Based on these data, electroporation conditions of 40 V/mm, and two bipolar 3.5 ms pulses were used with Cas9:sgRNA RNP targeting the Bmpr2 locus. With the Bmpr2 guide-RNA (gRNA), similar results of a 100% (n=8) mutation rate and 18% (n=461; control 18%, n=50) blastocyst development rate were observed.

### 3. Mosaicism Analysis

Individual bovine (n=157) and ovine (n=40) blastocysts electroporated with two bipolar 3 ms pulses at 20 and 30 V/mm, respectively, with guides targeting H11 locus were prepared for NGS to evaluate allelic variation and rates of mosaicism. The median bovine mutation efficiency per blastocyst was 98.5% with a mean of 88.8% (number of reads=54,709; FIG. 1A). Seventy-five percent of the blastocysts had editing rates >90%. Similarly, the median ovine mutation efficiency per blastocyst was 98.5% with a mean of 90%, and 87% had editing rates >90% (number of reads=37,368; FIG. 1B).

The majority (~99%) of amplicons from a NGS analysis did not amplify with the unique barcode preventing the analysis of all the allelic variants at the individual blastocyst level. However, these barcode-unmatched reads could be pooled for each species and gRNA group to analyze the types of mutations. The total mutation efficiency in the barcode-unmatched bovine and ovine blastocyst reads was 90% (n=7,385,901) and 94.7% (n=2,187,527), respectively. The types of allelic variants that were found in bovine and ovine blastocysts are illustrated in FIGS. 2 and 3, respectively.

The most common variants, explaining >50% of the mutations, were small 1-6 bp deletions or insertions located from 4 bp downstream to 1 bp upstream relative to the predicted cut site. There was more variability with the alleles observed in the bovine group with 11 different mutations making up half of the edit outcomes (FIG. 2), as compared with sheep where only 5 mutations represented over half of the outcomes (FIG. 3). The Shannon entropy of mutational outcomes for the two targets:

$$- \sum {p}_{i} * log ( {p}_{i} ) \qquad ( eq . 1 )$$

where pᵢ is the frequency of ith indel of that target, was slightly higher for cattle (1.39) than for sheep (1.26) and this can be seen by the more varied editing outcomes observed in FIG. 2.

Tables 4A and 4B display allelic variants present at a frequency of 5% are displayed for the 30 (A) bovine and (B) ovine electroporated blastocysts with the most NGS barcode-matched reads. Sequence number represents the number of reads for each blastocyst.

The 30 blastocysts with the most reads from each species were selected for detailed mosaicism analysis. Sequence data from one out of the 30 bovine blastocysts analyzed comprised mostly wild-type sequence (Table 4A). Thirteen percent (4/30) of the blastocysts comprised single mutations with very few (<5%) reads showing other mutant alleles, 33% (10/30) had two different mutant allele types, and ~50% of the blastocysts harbored more than two genetically distinct alleles, suggesting that genetic mosaicism is a common outcome from this approach.

Electroporated ovine zygotes resulted in an average mutation rate of 89.78% per embryo, and none of the 30 blastocysts analyzed were found to be comprising mostly the reference sequence (Table 4B). The distribution of types of edits was similar to the cattle with evidence of single mutations (5/30), two different mutant allele types (17/30), and 26.5% (8/30) harboring more than two genetically distinct alleles. The proportion of mosaic blastocysts harboring more than two genetically distinct alleles was not significantly different between the two species, x² (1, N=60)=1.78, p=0.18.

TABLE 4A
displays allelic variants present at a frequency of ‡5% are displayed for the 30 bovine
electroporated blastocysts with the most NGS barcode-matched reads. Sequence number
represents the number of reads for each blastocyst.

| Sequence # | 463 | 493 | 485 | 455 | 469 | 451 | 425 | | |
|---|---|---|---|---|---|---|---|---|---|
| no variant | | | 8% | | | 9% | | | |
| −2:1D | | 30% | 28% | 23% | | 10% | | | |
| −4:6D | | | | | | 7% | | | |
| −222:229D | | | | | | | | | |
| −5:4D | | | 47% | 21% | 5% | 5% | | | |
| −187:198D | | | | | | | | | |
| −85:83D | | | | | | | | | |
| −174:199D | | | | | | | | | |
| −97:100D | | | | | | | | | |
| −8:11D | | | | 23% | | | | | |
| −2:159D | | | | | | | | | |
| −2:2D | | | | | | | | | |
| −1:2D | | | | | | | | | |
| −146:151D | | | | | | | | | |
| −11:12D | | | | | | | | | |
| −4:7D | 88% | | | | | | | | |
| 1:1I | | | | | | | | | |
| −3:5D | | | | | | 8% | | | |
| −1:3D | | | | | | | | | |
| −5:22D | | 58% | | | | | | | |
| −1:2I | | | | | | | | | |
| 1:1D | | | | | | | | | |
| −95:106D | | | | | | | | | |
| −4:5I | | | | | 52% | | | | |
| −2:1I | | | | | | | | | |
| −10:10D | | | | | | | 47% | | |
| −19:19D | | | | | | | | | |
| −24:165D | | | | | | | | | |
| −142:147D | | | | | | | | | |
| −20:21D | | | | | | | | | |
| 1:4I | | | | | | | | | |
| −3:8D | | | | | | | | | |
| −5:16D | | | | | | | | | |
| −1:105D | | | | | | | | | |
| −2:3D | | | | | | | | | |
| −183:183D | | | | | | | | | |
| −43:45D | | | | | | | | | |
| −5:49D | | | | | | | 25% | | |
| −7:15D | | | | | | | | | |
| −12:56D | | | | | | | | | |
| −7:12D | | | | | | 22% | | | |
| −16:18D | | | | | 13% | | | | |
| Other | | | | | | | | | |
| Sequence # | 546 | 509 | 396 | 596 | 593 | 432 | 672 | | |
| no variant | 15% | | | | | 19% | | | |
| −2:1D | | 38% | | | | 8% | | | |
| −4:6D | | | | | | | | | |
| −222:229D | | | | | | | | | |
| −5:4D | | 40% | | | | | 35% | | |
| −187:198D | | | | | | | | | |
| −85:83D | | | | | | | | | |
| −174:199D | | | | | | | | | |
| −97:100D | | | | | | | | | |
| −8:11D | | | | | | | | | |
| −2:159D | | | | | | | | | |
| −2:2D | | | | | | | | | |
| −1:2D | | | | 82% | | | | | |
| −146:151D | | | | | | | | | |
| −11:12D | | | | | | | | | |
| −4:7D | | | | | | | | | |
| 1:1I | | | | | | | | | |
| −3:5D | | | | | 55% | | | | |
| −1:3D | | | | | | | | | |
| −5:22D | | | | | | | | | |
| −1:2I | | | | | | | | | |
| 1:1D | | | | | | | | | |
| −95:106D | | | | | | | | | |
| −4:5I | | | | | | | | | |
| −2:1I | | | | | | | | | |
| −10:10D | | | | | | | | | |
| −19:19D | | | | | | 9% | | | |
| −24:165D | | | | | | | | | |
| −142:147D | | | | | | 47% | | | |
| −20:21D | | | | | | | 26% | | |
| 1:4I | | | | | 28% | | | | |
| −3:8D | 29% | | | | | | | | |
| −5:16D | | | | | | | | | |
| −1:105D | | | 37% | | | | | | |
| −2:3D | | | | | | | | | |
| −183:183D | | | 7% | | | | | | |
| −43:45D | 20% | | | | | | | | |
| −5:49D | | | | | | | | | |
| −7:15D | 18% | | | | | | | | |
| −12:56D | | | | | | | | | |
| −7:12D | | | | | | | | | |
| −16:18D | | | 16% | | | | | | |
| Other | | | | | | | | | |
| Sequence # | 447 | 699 | 508 | 592 | 964 | 897 | 955 | | |
| no variant | | | 6% | | 19% | | | | |
| −2:1D | | | | | 5% | | | | |
| −4:6D | | 64% | | 20% | | | 15% | | |
| −222:229D | | | 61% | | | | | | |
| −5:4D | | | 9% | | 5% | 17% | | | |
| −187:198D | | | | | | | | | |
| −85:83D | | | | | | | | | |
| −174:199D | | | | | | | | | |
| −97:100D | | | | | | | 78% | | |
| −8:11D | | | | | | 16% | | | |
| −2:159D | 65% | | | | | | | | |
| −2:2D | | | | | | | | | |
| −1:2D | | | | | | | | | |
| −146:151D | | | | | | | | | |
| −11:12D | | | | | | | | | |
| −4:7D | | | | | | | | | |
| 1:1I | | | | | | | | | |
| −3:5D | | | | | | | | | |
| −1:3D | | | | | | 41% | | | |
| −5:22D | | | | | | | | | |
| −1:2I | | | | | 29% | | | | |
| 1:1D | | | | | | | | | |
| −95:106D | | | | 42% | | | | | |
| −4:5I | | | | | | | | | |
| −2:1I | | | | | 15% | | | | |
| −10:10D | | | | | | | | | |
| −19:19D | | | | 27% | | | | | |
| −24:165D | | | | | | | | | |
| −142:147D | | | | | | | | | |
| −20:21D | | | | | | | | | |
| 1:4I | | | | | | | | | |
| −3:8D | | | | | | | | | |
| −5:16D | 16% | | | | | | | | |
| −1:105D | | | | | | | | | |
| −2:3D | | 15% | | | | | | | |
| −183:183D | | | | | | | | | |
| −43:45D | | | | | | | | | |
| −5:49D | | | | | | | | | |
| −7:15D | | | | | | | | | |
| −12:56D | | | | | | | | | |
| −7:12D | | | | | | | | | |
| −16:18D | | | | | | | | | |
| Other | | | | | | | | | |
| Sequence # | 944 | 884 | 947 | 814 | 1723 | 985 | 1700 | 1821 | 3593 |
| no variant | 74% | | | | | | | | |
| −2:1D | | 19% | | | 28% | | 15% | | |
| −4:6D | | | | | 40% | | | | |
| −222:229D | | 51% | | | | | | 40% | |
| −5:4D | | | | | | | | | |
| −187:198D | | | | | | 92% | | | |
| −85:83D | | | | | | | | | 88% |
| −174:199D | | | | | | | | 15% | |
| −97:100D | | | | | | | | | |
| −8:11D | | | 30% | | | | | | |
| −2:159D | | | 18% | | | | | | |
| −2:2D | | | | | 27% | | | | |
| −1:2D | | | | | | | | | |
| −146:151D | | | | 59% | | | | | |
| −11:12D | | | 18% | | | | | | |
| −4:7D | | | | 28% | | | | | |
| 1:1I | | | | | | | | | |
| −3:5D | | | | | | | | | |
| −1:3D | | | | | | | | | |
| −5:22D | | | | | | | | | |
| −1:2I | | | | | | | | | |
| 1:1D | | 28% | | | | | | | |
| −95:106D | | | | | | | | | |
| −4:5I | | | | | | | | | |
| −2:1I | | | | | | | | | |
| −10:10D | | | | | | | | | |
| −19:19D | | | | | | | | | |
| −24:165D | | | 22% | | | | | | |
| −142:147D | | | | | | | | | |
| −20:21D | | | | | | | | | |
| 1:4I | | | | | | | | | |
| −3:8D | | | | | | | | | |
| −5:16D | | | | | | | | | |
| −1:105D | | | | | | | | | |
| −2:3D | | | | | | | | | |
| −183:183D | | | | | | | | | |
| −43:45D | | | | | | | | | |
| −5:49D | | | | | | | | | |
| −7:15D | | | | | | | | | |
| −12:56D | | | | | | | | | |
| −7:12D | | | | | | | | | |
| −16:18D | | | | | | | | | |
| Other | | | | | | | 83% | 10% | |

TABLE 4B
displays allelic variants present at a frequency of ‡5% are displayed for the 30 ovine
electroporated blastocysts with the most NGS barcode-matched reads. Sequence number
represents the number of reads for each blastocyst.

| Sequence # | 2384 | 1313 | 1502 | 1086 | 292 | 314 | 307 | 484 | 740 |
|---|---|---|---|---|---|---|---|---|---|
| no variant | | | | | | | | | |
| −5:4D | | 64% | | | | 46% | | 6% | |
| −4:6D | 25% | | 9% | 93% | | | | | |
| −2:1D | | | | | | | 57% | | |
| 1:1I | 12% | | | | 29% | | 34% | 41% | |
| −1:2I | 57% | 27% | | | | | | | |
| −5:5D | | | | | | 36% | | | |
| −8:11D | | | | | | | | | |
| −12:10D | | | | | | | | | |
| 1:1D, | | | | | | | | | |
| 8:19D | | | | | | | | | |
| −4:2D | | | | | | | | | |
| −11:62D | | | | | | | | | |
| SNV:−1T, | | | | | | | | | |
| 1A, 2C | | | | | | | | | |
| −39:53D | | | | | | | | | |
| −111:111D | | | | | 63% | | | | |
| SNV:1A | | | | | | | | | |
| −2:1I | | | | | | | | | |
| −1:5I | | | | | | | | | 46% |
| −3:6D, | | | | | | | | | |
| 9:19D | | | | | | | | | |
| 1:2D | | | | | | | | | 48% |
| −16:18D | | | 44% | | | | | | |
| −9:16D | | | | | | | | | |
| −2:3I | | | 41% | | | | | | |
| 1:3D | | | | | | | | | |
| −8:9D | | | | | | | | 39% | |
| 5:4I | | | | | | | | | |
| −2:2D | | | | | | | | | |
| −6:9D | | | | | | | | | |
| Sequence # | 1132 | 794 | 1273 | 2017 | 2550 | 437 | 1248 | 460 | 709 |
| no variant | | 22% | | | | | | 5% | 6% |
| −5:4D | 36% | 16% | 11% | | | 68% | | | |
| −4:6D | | | 19% | | | | 93% | | |
| −2:1D | | | | | | 24% | | 88% | |
| 1:1I | | | | | | | | | |
| −1:2I | | 38% | | | | | | | |
| −5:5D | | 5% | | | | | | | |
| −8:11D | | | | | | | | | |
| −12:10D | | | | | 96% | | | | |
| 1:1D, | | | | 89% | | | | | |
| 8:19D | | | | | | | | | |
| −4:2D | | | | | | | | | |
| 11:6−2D | | | 61% | | | | | | |
| SNV:-1T, | | | | | | | | | 77% |
| 1A, 2C | | | | | | | | | |
| −39:53D | | | | | | | | | |
| −111:111D | | | | | | | | | |
| SNV:1A | | | | | | | | | |
| −2:1I | 54% | | | | | | | | |
| −1:5I | | | | | | | | | |
| −3:6D, | | | | | | | | | |
| 9:19D | | | | | | | | | |
| 1:2D | | | | | | | | | |
| −16:18D | | | | | | | | | |
| −9:16D | | | | | | | | | |
| −2:3I | | | | | | | | | |
| 1:3D | | | | | | | | | |
| −8:9D | | | | | | | | | |
| 5:4I | | | | | | | | | |
| −2:2D | | | | | | | | | |
| −6:9D | | | | | | | | | |
| Sequence # | 379 | 784 | 327 | 828 | 1759 | 741 | 2892 | 1342 | 1442 |
| no variant | 7% | | | 7% | | | | | |
| −5:4D | | | 74% | | 91% | | | | |
| −4:6D | | | | | | 55% | | 43% | |
| 2:1D | 80% | | | | | | 91% | | |
| 1:1I | | | | 22% | | | | | |
| −1:2I | | | | | | | | | |
| −5:5D | | | | | | | | | |
| −8:11D | | | | | | | 5% | 45% | 48% |
| −12:10D | | | | | | | | | |
| 1:1D, | | | | | | | | | |
| 8:19D | | | | | | | | | |
| −4:2D | | 40% | | | | 42% | | | |
| −11:62D | | | | | | | | | |
| SNV:−1T, | | | | | | | | | |
| 1A, 2C | | | | | | | | | |
| −39:53D | | | | 67% | | | | | |
| −111:111D | | | | | | | | | |
| SNV:1A | | | | | | | | | |
| −2:1I | | | | | | | | | |
| −1:5I | | | | | | | | | |
| −3:6D, | | 51% | | | | | | | |
| 9:19D | | | | | | | | | |
| 1:2D | | | | | | | | | |
| −16:18D | | | | | | | | | |
| −9:16D | | | | | | | | | 43% |
| −2:3I | | | | | | | | | |
| 1:3D | | | | | | | | | |
| −8:9D | | | | | | | | | |
| 5:4I | | | | | | | | | |
| −2:2D | | 20% | | | | | | | |
| −6:9D | | | | | | | | | |
| Sequence # | 1489 | 1208 | 2236 | | | | | | |
| no variant | 26% | | | | | | | | |
| −5:4D | 13% | 67% | 29% | | | | | | |
| −4:6D | 43% | | | | | | | | |
| −2:1D | 9% | | | | | | | | |
| 1:1I | | | | | | | | | |
| −1:2I | | | | | | | | | |
| −5:5D | | | | | | | | | |
| −8:11D | | | | | | | | | |
| −12:10D | | | | | | | | | |
| 1:1D, | | | | | | | | | |
| 8:19D | | | | | | | | | |
| −4:2D | | | | | | | | | |
| −11:62D | | | | | | | | | |
| SNV:−1T, | | | 58% | | | | | | |
| 1A, 2C | | | | | | | | | |
| −39:53D | | | | | | | | | |
| −111:111D | | | | | | | | | |
| SNV:1A | | | | | | | | | |
| −2:1I | | | | | | | | | |
| −1:5I | | | | | | | | | |
| −3:6D, | | | | | | | | | |
| 9:19D | | | | | | | | | |
| 1:2D | | | | | | | | | |
| −16:18D | | | | | | | | | |
| −9:16D | | | | | | | | | |
| −2:3I | | | | | | | | | |
| 1:3D | | | | | | | | | |
| −8:9D | | | | | | | | | |
| 5:4I | | 28% | | | | | | | |
| −2:2D | | | | | | | | | |
| −6:9D | | | | | | | | | |

4. rAAV Serotype Optimization

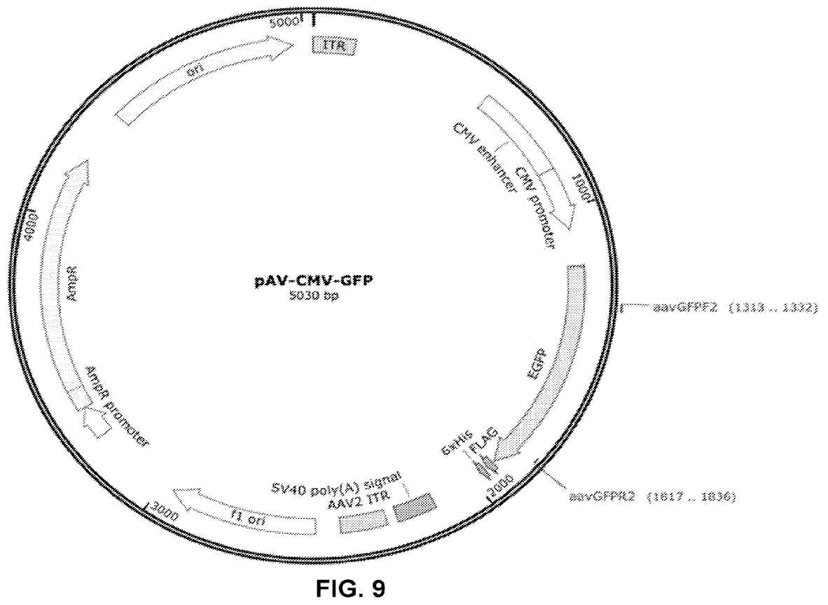

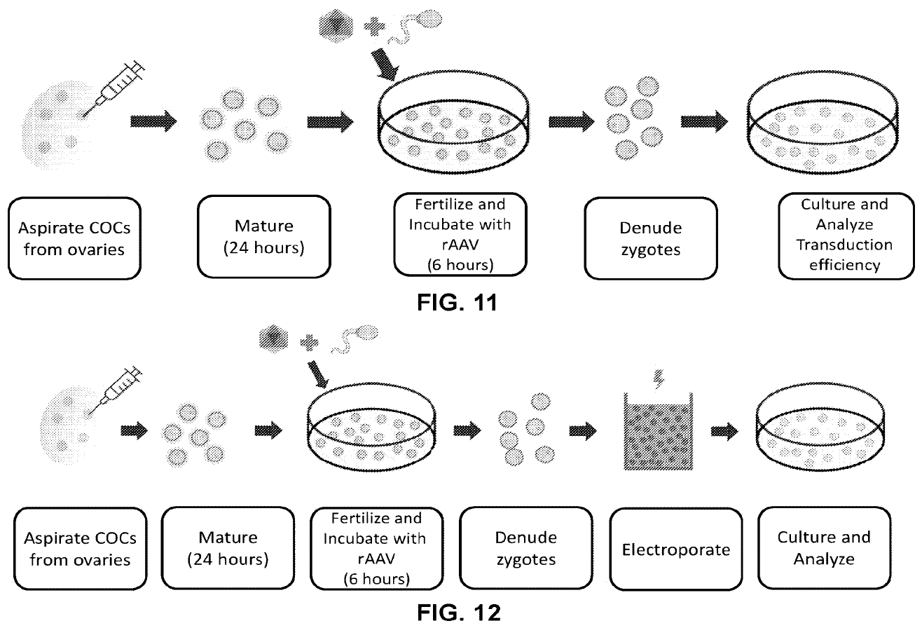

rAAV serotypes 1, 2, 5, 6, 8, and 9 were tested for transduction efficiency in bovine zygotes during fertilization. Based on the methods by Chen et al. (Ref. 22; FIG. 11) matured oocytes were incubated for 6 h with sperm as well as various concentrations of rAAV for the delivery of a CMV-eGFP reporter gene (FIG. 9).

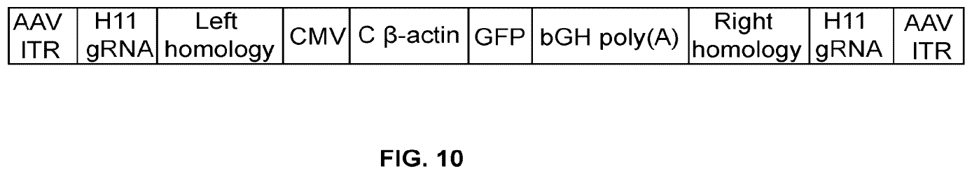

HDR donor template. A CMV-eGFP reporter plasmid as shown in FIG. 9 was obtained from Charles River, Rockville, MD (AAV GFP Testing Kit, catalog #CT0002, listed as “pAV-CMV-GFP”). The plasmid contained a 3.9 kb HDR donor template containing 600 bp H11 homology arms with gRNA target sites at the ends, the CAG promoter, GFP gene with a nuclear localization signal, and rAAV2 ITR arms as shown in FIG. 10.

Serotype 6 was tested at concentrations of 10⁶-10¹¹viral genome copies (vgc) in 50 lL of synthetic oviductal fluid-In vitro fertilization (SOF-IVF) medium. Blastocyst rates were 54% (n=26), 4400 (n=25), 4800 (n=21), 47% (n=17), 30% (n=27), and 32% (n=28) for 106-1011 vgc, respectively, as compared with control 50% (n=22).

Serotype 6 showed efficient transduction as evidenced by both GFP expression (FIGS. 4A-4C) and polymerase chain reaction (PCR) amplification of the CMV-eGFP reporter in blastocysts. Approximately 380% (n=32) for 10¹⁰ vgc and 3300 (n=27) for 10¹¹ vgc of the treated zygotes that developed into blastocysts expressed bright fluorescence, and the presence of CMV-eGFP reporter gene in all 27 embryos was confirmed by PCR amplification. Embryos treated with this concentration of rAAV, however, did seem to have a reduced blastocysts development rate when compared with controls. All other serotypes tested did not result in visible expression of GFP. Based on these results, rAAV6 was used for further experiments.

### 5. Targeted Insertion of GFP

Initially, a KI primary granulosa cell line was produced using lipofection of the rAAV6 plasmid containing the 3.9 kb HDR donor template (FIG. 10) and Cas9: sgRNA RNP targeting the H11 locus to serve as a KI positive control. PCR amplification of the right and left junctions (Table 5) was utilized to confirm a targeted KI of the donor template.

TABLE 5
Sequence of primers used for PCR amplification of the bovine H11 region and HDR
knock-ins.

| | | | | | Product | SEQ ID |
|---|---|---|---|---|---|---|
| | Region | Primer | Sequence | Target | Size (bp) | NO: |
| Knock-In | H11 | 1a | TGCCACTGTTGCTTGAGACT | 5′ | 1083 | 23 |
| | H11 | 1b | CCAAGTGGGCAGTTTACCGT | junction | | 24 |
| | H11 | 2a | TGCTGGGATTACACATGGCA | 3′ | 1444 | 25 |
| | H11 | 2b | AAGCACGGCCTAGTGGAGAA | junction | | 26 |
| Wild | H11 | 1a | TGCCACTGTTGCTTGAGACT | H11 | 1954 | 23 |
| Type | H11 | 2b | AAGCACGGCCTAGTGGAGAA | | | 25 |
| | H11 | H11F2 | CCCCAGTGTTGTGCATGTAG | | 505 | 27 |
| | H11 | H11R2 | GTGAATGCCACTGCTGTGTT | | | 28 |
| Insert | Donor | aavGFPF | ATGGTAATCGTGCGAGAGGG | GFP | 560 | 29 |
| | Template | | | | | |
| | Donor | aavGFPR | GGCCACGGAACAGGTAGTTT | | | 30 |
| | Template | | | | | |

Primer legend: bH11LjuncF2 (1a); bH11LjuncR2 (1b); bH11RjuncF2 (2a); and bH11RjuncR2 (2b).

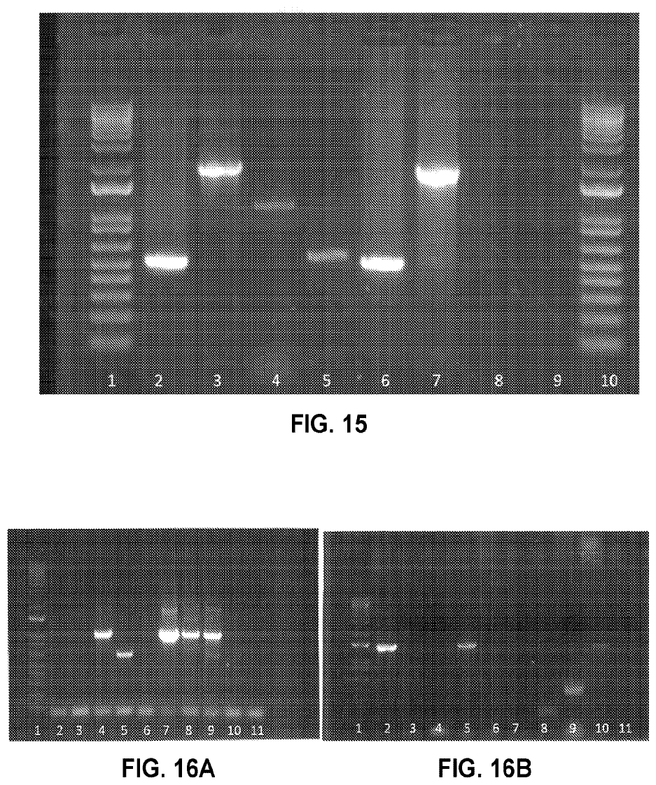

Primers targeting the bovine genome outside of the targeted gene insertion (Table 5) showed that there were also cells in this primary line that did not have the targeted KI as the smaller wild-type sequences were preferentially amplified (FIG. 15).

To produce bovine blastocysts containing a large template KI using rAAV6 and electroporation, a 3.9 kb HDR donor template containing 600 bp H11 homology arms with gRNA target sites flanking the ends, the CMV enhancer, the CAG promoter, GFP gene with a beta-actin nuclear localization signal, and rAAV2 ITR arms was packaged into rAAV6 (FIG. 01). After this, matured cumulus-oocyte complexes (COCs) were incubated with the rAAV6 containing the HDR donor template at different concentrations, and sperm for 6 h before denuding and electroporating with Cas9:sgRNA RNP targeting the H11 locus (FIG. 12).

However, no blastocysts expressing GFP were observed. In addition, PCR amplification of blastocyst DNA using primers targeting the 5′ and 3′ junction of the targeted insertion did not result in a product, and primers amplifying the bovine H11 locus target site produced wild-type sized amplicons. It was observed that cumulus cells that remained after the post-fertilization denuding were expressing GFP (FIG. 5), suggesting that the cumulus cells surrounding the oocytes during incubation with rAAV6 were being transduced.

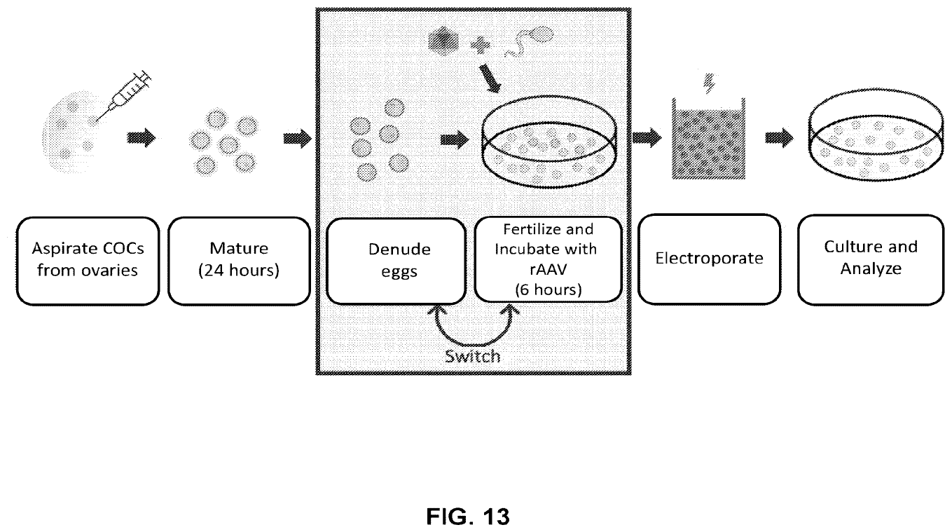

To improve transduction of the oocytes instead of the cumulus cells, an approach that required denuding the cumulus cells from the oocytes before incubation with rAAV6 and sperm was trialed (FIG. 13). In addition, and importantly, ~5 undenuded COCs for each 20 denuded oocytes were added to each drop to provide the environment to help ensure acceptable fertilization and subsequent blastocyst development rates.

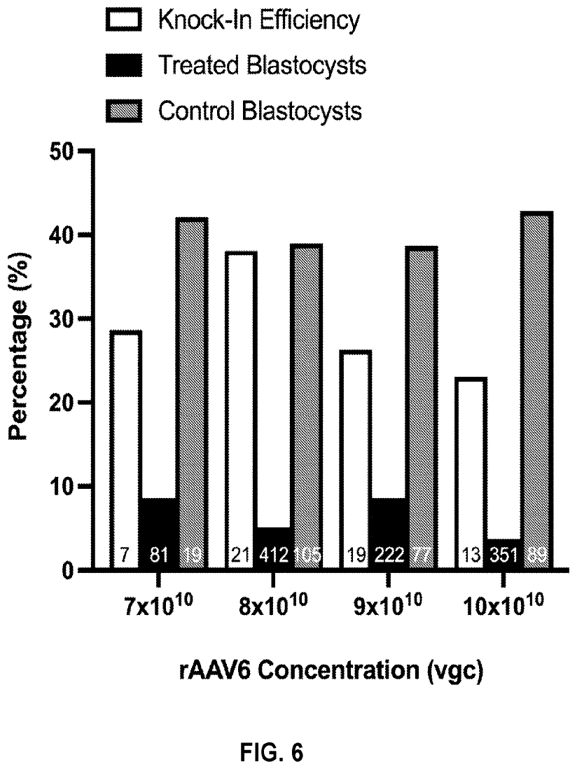

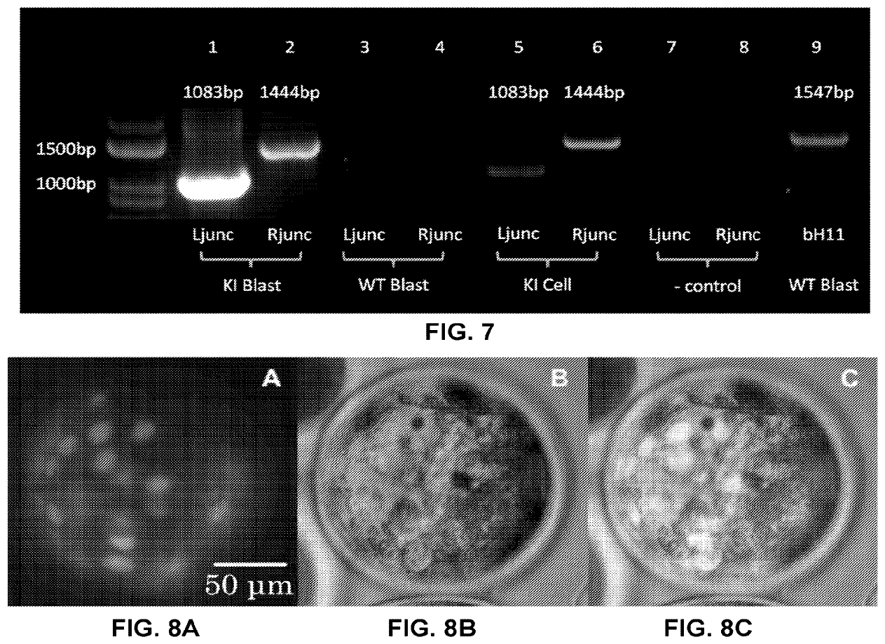

GFP-expressing blastocysts were successfully produced with this approach as confirmed by fluorescent imaging. Incubation of denuded oocytes with rAAV6 at concentrations of 7×10¹⁰, 8×10¹⁰, 9×10¹⁰, and 10¹¹ vgc in 50 lL of SOF-IVF medium produced GFP-expressing blastocysts (FIG. 6; Table 6). There were no GFP-expressing blastocysts produced at concentrations <5×10¹⁰ vgc, or >3×10¹¹ vgc. Targeted KIs were confirmed by PCR and Sanger sequencing (FIG. 7 and FIGS. 16A-16B).

TABLE 6
rAAV6 concentration in relation to blastocyst development and targeted knock-in rates.

| | | | | | | | Avg. | |
|---|---|---|---|---|---|---|---|---|
| | | | | | | | Ctl. | |
| | | | | | | Knock- | blasto- | Treated/control |
| rAAV | | | Green | PCR | Blastocysts/ | ins/ | cyst | blastocyst |
| Concen- | Embryos | Blasto- | Blasto- | knock- | Embryos | blasto- | rate | development |
| tration | treated | cysts | cysts | in | treated | cysts | (%) | ratio |
| 7 × 10¹⁰ | 81 | 7 | 3 | 2 | 8.6% | 28.6% | 42.1 | 0.20 |
| 8 × 10¹⁰ | 412 | 21 | 8 | 8 | 5.1% | 38.1% | 39.0 | 0.13 |
| 9 × 10¹⁰ | 222 | 19 | 5 | 5 | 8.6% | 26.3% | 38.7 | 0.22 |
| 1 × 10¹¹ | 351 | 13 | 3 | 3 | 3.7% | 23.1% | 42.8 | 0.09 |

However, when looking at the fluorescent images of knocked in blastocysts, it was observed that they were not uniformly green (FIGS. 8A-8C). FIGS. 8A-8C are images of GFP-expressing bovine blastocyst that underwent rAAV6 incubation and electroporation imaged on day 7 postfertilization using (A) a FITC filter, (B) bright field, and (C) merged (bright field+FITC).

To test whether one of the causes of uneven GFP expression in blastocysts containing confirmed targeted GFP integration was mosaicism, primers targeting the bovine genome outside of the targeted gene insertion (bH11WTF2, bH11WTR2; Table 5) were also used in a subset of the GFP-expressing blastocysts to identify whether wild-type sized sequence remained.

Wild-type sized alleles were identified in all of the five samples analyzed, suggesting that the fluorescent blastocysts were mosaic containing both non-KI H11 sequence and the targeted 2.7 kb GFP KI. Three out of the five wild-type sized H11 sequence contained small indels indicating cutting at the target site in addition to the targeted KI, and two contained unedited wild-type DNA sequence in addition to the targeted KI.

### C. Discussion

### 1. Electroporation

Gene-editing technologies offer an approach to introduce targeted genetic alterations in livestock genomes to augment traditional selective breeding approaches (Ref 32). Electroporation is a technique widely used in biotechnology and medicine for the delivery of drugs and genes into living cells. Electroporators work by directing pulses of electrical current to create transient (ms to min range) pores in the lipid bilayer of the plasma membrane that allows the passage of reagents into the cell. Electroporation allows for the simultaneous and instantaneous processing of upward of 100 zygotes with the push of a button making it a scalable and simple approach to producing gene-edited livestock (Ref. 3).

Species-specific optimization of electroporation parameters is necessary to achieve both a high survival rate and efficient editing of livestock zygotes. A general rule of thumb is that there is an inverse relationship between the factors that result in high rates of editing (increasing pulse voltage and number, and use of bipolar pulses) to increase the efficiency of macromolecule uptake through the membrane, and rates of embryo survival through to the blastocyst stage.

Bovine zygotes appear to be especially sensitive to high voltages, with 20 V/mm (3 pulses, 1 ms) resulting in lower blastocyst rates than 10 V/mm (Ref 16). Increasing the voltage strength to 45 V/mm (5 pulses, 3 ms) was associated with high rates of bovine zygote lysis, suggesting damage to the cell membrane lipid bilayer (Ref. 18). Similar results were also reported by Miao et al. (Ref 12) where pulses of 20, 25, and 30 V/mm had an increasingly negative impact on bovine blastocyst development rates. In this experiment, high rates of mutation and acceptable embryo viability were observed when electroporating Cas9:sgRNA RNP targeting the H11 locus using three bipolar 3 ms 20 V/mm poring pulses. The same settings were also used to target the Rosa26 locus that similarly yielded high mutation and acceptable development rates.

In sheep embryos, high rates of editing and good embryo viability were obtained using two 3.5 ms 40 V/mm bipolar poring pulses. This is twice the poring voltage that was found to be optimal for bovine zygotes, which could be influenced by the size of the zygote. Bovine oocytes and zygotes are larger (~150 μm diameter) than those of sheep and goat (~120 μm) (Ref. 33), which are in turn larger than those of rats and mice (~70 μm). Electroporation of rat and mouse zygotes has been shown to be efficient with high poring voltages ~40-50 V/mm, with acceptable development rates. It is known that membrane permeabilization can be achieved at lower voltages on larger cells as compared with what is required for smaller cells (Ref 15).

The barcode-unmatched NGS sequences could not be analyzed for mutation rates and alleles for individual blastocysts. The failure of the attachment of DNA barcodes may have been due to the overamplification of the first PCR amplification when using target-specific primers, the presence of target-specific primers in the second PCR amplification, or the failure of barcoding primers to anneal. However, the barcode-unmatched sequences could still be analyzed as a group based on species (FIGS. 2 and 3).

The cattle and sheep barcode-unmatched sequences revealed similar results, with 90% or more of the reads containing mutations. There was a little more variability in the edits seen in the cattle blastocysts than the sheep blastocysts, which is reflected in the slightly higher Shannon entropy of mutational outcomes figure for the bovine blastocysts. One weakness of the way this assay was performed is that large deletions that ablated the PCR primer sites would not have amplified, and so all deletion events may not have been captured.

A comprehensive study in 2019 developed a logistic regression model to predict insertions and deletions that result from CRISPR/Cas9-mediated cleavage of an arbitrary sequence based on >1 million mutational events resulting from CRISPR/Cas9-mediated cleavage and nonhomologous end joining-mediated double-strand break repair of 6872 synthetic target sequences (Ref. 34). Similar to this study, they found that ~9.8% of their targets were unedited, and of the edited sequences, 63.6% were deletions, and 31.5% were insertions.

Interestingly, the three most common mutations predicted by their model based on the bovine and ovine sequences (−2:4D, −1:1D, −1:1I) were not among the top three mutations observed in bovine (−2:1D, −5:4D, −4:6D; FIG. 2) or ovine (−5:4D, −4:6D, −2:1D, FIG. 3) blastocysts in this study.

NGS analysis of matched-barcode electroporated bovine and ovine zygotes revealed high rates of mutation, suggesting that electroporation can be used as a high throughput approach to generate genome-edited livestock, however, many embryos were found to contain more than two genetically distinct alleles indicating mosaicism. This is likely due to nuclease activity after the first cell division, which can be caused by prolonged nuclease activity even after various cell divisions. Genetic mosaicism is not an issue when producing animals with short generational intervals such as mice, since the unwanted alleles can be quickly bred out. Mosaicism within livestock species, however, poses an issue as long generational intervals make breeding unwanted alleles out at a large scale a prohibitively expensive and unrealistic task.

### 2. 2.7 kb GFP Targeted KI

According to the present example, serotype rAAV6 was found to transduce DNA fragments into zygotes without treatment to weaken the ZP, in agreement with murine studies (Refs. 20,22,28,29). A recent study evaluated five different AAV serotypes for their ability to deliver genetic material into bovine zygotes by placing them in a culture medium that used 5×10⁹ vgc per 100 μL of culture medium (Ref. 35). This study also found AAV6 was able to transduce reporter DNA into bovine embryos, but additionally in that experiment equivalent efficiencies were seen with AAV1, AAV2, AAV6, and AAV-DJ, whereas AAV9 was found to be less efficient. In that experiment, the authors reported that rate of blastocyst formation was similar between the AAV-treated (12.5-23.5%) and control (22%) groups.

This same research group further used AAV2 at 5×10¹⁰ vgc per 100 μL of culture medium to deliver Cas9 coding sequences (3159 bp) and sgRNA targeting the gene CD209 expressed by the U6 promoter in one virus particle. Three of 22 resulting blastocysts (13%) were edited, however, these embryos were mosaic CD209 frameshifts. The authors explained this due to the delay in Cas9 activity resulting from the time taken for the saCas9 gene to be transcribed and translated. As discussed previously, mosaicism is particularly problematic in livestock embryos due to the length of time required to produce a true breeding line.

In this experiment, rAAV transduction of a 3.9 kb HDR donor repair template in combination with electroporation of Cas9:sgRNA RNP complexes into zygotes was sufficient for the generation of bovine blastocysts with a large-targeted KI. The ability of rAAV to package DNA fragments of up to 4.9 kb and transduce various cell types, while being nonpathogenic, makes it an attractive vector for delivering HDR templates into early-stage embryos. It significantly lowers the technical barrier and conceptually reduces the amount of specialized equipment required for producing large KI animals.

However, it should be noted that denuding the oocytes before incubation with rAAV6 and sperm was necessary to successfully produce targeted KIs. When rAAV was incubated with nondenuded oocytes during fertilization, the cumulus cells were transduced by rAAV apparently reducing the amount of HDR template introduced into the oocyte. As a result, cumulus cells expressed GFP, but no GFP-expressing blastocysts were observed.

Of the various concentrations tested, 8×10¹⁰ vgc in 50 μL of SOF-IVF medium resulted in the most efficient KI rate of approximately 38% of those embryos that developed to the blastocyst stage. In mice, a 771 bp cassette delivered to morulas ex vivo by rAAV6 was knocked in by Cas9-mediated HDR in blastocysts at rates as high as 57%. However, a much lower percentage of liveborn mice resulting from embryo transfer carried a KI, and high rates of mosaicism were observed in a way that was inversely correlated with the concentration of the rAAV dose (Ref. 29).

Similarly, according to the present example, the KI embryos were visibly mosaic (FIGS. 8A-8C), and the presence of both wild-type and edited target sequences was confirmed by PCR. To circumvent transduction of the cumulus cells, they were removed by denuding the COCs before rAAV incubation. Unfortunately, this negatively impacted embryo development as denuding before incubation with sperm has been shown to significantly decrease maturation, fertilization, and embryo development (Ref 36). Rates of embryonic development to the blastocyst stage were significantly decreased in cumulus-denuded oocyte (Ref. 37).

To improve the embryo development of denuded oocytes, 5 COCs were added to each drop of 20 denuded oocytes to provide factors secreted by cumulus cells (Ref. 38). The blastocyst development rate for denuded transduced embryos was 3.7-8.6% (Table 3) as compared with an average of ~39% for control embryos. This low rate of blastocyst development would likely render the implementation of this approach infeasible for most commercial circumstances, where blastocyst development rate from oocytes in vitro matured, fertilized, and cultured in vitro is typically in excess of 30%. Under typical conditions, 20% to 40% of in vitro cultured bovine zygotes will reach the blastocyst stage (Ref 39).

One approach that could maintain high rates of blastocyst development would be to incubate the zygotes with rAAV following denuding after fertilization or at later stages of development, however, previous study has shown high rates of mosaicism when introducing gene-editing reagents later than 6-8 hpi (Ref 40) with a 100% mosaicism rate being observed when microinjecting editing reagents into denuded embryos at 20 hpi. In mice, targeted in vivo gene editing was achieved in 3 out of 29 (10%) pups born after direct injection of rAAV particles containing CRISPR-Cas9 components into the ampulla of a single oviduct after fertilization. At this stage, the zygote is considered embryonic day 0.5 (E0.5) or around 12 hpi since mating is assumed to have occurred at the midpoint of the dark period prior to the morning plug detection.

Some have envisaged that rAAV transduction and zygote electroporation of gene-editing reagents might enable on-farm editing of livestock species (Ref. 1). However, the mosaicism observed in the editing step, combined with that seen in the KI step, suggests further optimization or an alternative approach will be required to produce nonmosaic large KI in developing embryos for livestock applications. One study simulating the commercial implementation of gene editing found that the level of mosaicism had a greater impact than gene-editing efficiency and zygote survival on the time required to fix desired edits, and the rate of genetic gain in the breeding program (Ref. 41).

Nonmosaic embryos could be generated from embryonic stem cells derived from the inner cell mass of chimeric blastocysts, with selected KI colonies serving as nuclei donors for the production of cloned blastocysts (Ref. 42). However, this approach negates the objective of achieving KI in embryos at scale using the combination of electroporation and rAAV. It remains to be seen whether the costs of implementing nuclear transfer cloning of edited cells, including the requisite technical expertise, time, and sophisticated equipment required, outweigh the considerable benefit of generating non-mosaic offspring of a known genotype and 100% germline transmission in commercial breeding settings.

### D. Materials and Methods

1. gRNA Design

A single gRNA targeting the bovine H11 locus (TAGCCATAAGACTACCTAT; SEQ ID NO:31) was designed as described in Hennig et al. (Ref. 43). Guide RNAs targeting the Rosa26 locus in the bovine genome (TGTCGAGTCTCGATTATGGG; SEQ ID NO:32) were designed as described in Yuan et al. (Ref. 44). A single gRNA targeting the H11 locus in the ovine genome (TAGCCACAAGACTACCTAT; SEQ ID NO:33) and Bmpr2 (CAATTCAGAATGGAACGTAC; SEQ ID NO:34) was designed using CHOPCHOP (Ref. 45) with no less than three mismatches in the guide sequence for off-target sites and at least one mismatch in the seed region (8-11 bp upstream of the protospacer adjacent motif (PAM) sequence) when compared with the reference genome (Ovis aries; Oar_v3.1).

Guides were then commercially synthesized (Synthego, Redwood City, CA) and confirmed to cut in vivo by cytoplasmic microinjection of in vitro fertilized embryos with 6 μL of a solution containing 67 ng/μL of gRNA alongside 167 ng/μL of Cas9 protein (PNA Bio, Thousand Oaks, CA) incubated at room temperature for 30 min before injection.

### 2. Embryo Production

Bovine and ovine ovaries were collected from local slaughterhouses and transported to the laboratory in 38.5° C. sterile saline. Upon arrival, COCs were aspirated from follicles, washed, and placed into 400 μL of equilibrated Bovine oocyte-in vitro maturation medium (BO-IVM) (IVF Biosciences, Falmouth, United Kingdom). Bovine and ovine COCs were incubated in BO-IVM medium for 20 hr at 38.5° C. in a humidified 5% C02 incubator. Groups of 25 matured COCs were then transferred into 50 μL drops of SOF-IVF and incubated with 2×10⁶ sperm per mL for 6 h at 38.5° C. in a humidified 5% C02 incubator for fertilization.

After 6 h of incubation with sperm, presumptive zygotes were denuded by vortex in SOF-N-2-hydroxyethylpiperazine-N′-2-ethanesulfonic acid (HEPES) for 5 min and cultured in bovine embryo-in vitro culture medium (BO-IVC)(IVF Biosciences, Falmouth, United Kingdom) at 38.5° C. in a humidified hypoxic atmosphere of 5% C02, 5% 02, and 90% N2 for 7 days. For ovine parthenogenetic activation, matured COCs were denuded by vortex in SOF-HEPES for 3 min before activation. Denuded oocytes underwent parthenogenetic activation and were incubated in BO-IVC medium supplemented with 6-dimethylaminopurine for 4 h. Oocytes were electroporated immediately after the 4-h incubation, and then cultured in BO-IVC medium at 38.5° C. in a humidified hypoxic atmosphere of 5% C02, 5% 02, and 90% N2 for 7 days.

### 3. Electroporation of Bovine and Ovine Zygotes and Parthenogentically-Activated Ovine Oocytes

Groups of 30-100 presumptive zygotes or activated oocytes were washed three times in Opti-MEM solution (Thermo Fisher Scientific, Waltham, MA) and transferred into a 1 mm electroporation cuvette (Bulldog Bio, Portsmouth, NH) along with 20 μL of electroporation solution containing Cas9:sgRNA RNP complexes, and Opti-MEM. Single gRNA and Cas9 protein concentrations were 100 and 200 ng/μL, respectively. Electroporation was performed using the Super Electroporator NEPA 21 (NEPA GENE Co. Ltd., Chiba, Japan) with various parameters as listed in Tables 2 and 3.

After electroporation, presumptive bovine and ovine zygotes were recovered and washed with SOF-HEPES followed by equilibrated BO-IVC and cultured in 400 μL of BO-IVC medium at 38.5° C. in a humidified atmosphere of 5% C02, 5% 02, and 90% N2 for 7 days.

4. rAAV Serotype Optimization

Oocytes were collected and matured as already described. Groups of 25 matured COCs were transferred into 50 μL drops of SOF-IVF to be incubated with sperm and various concentrations of rAAV containing a CMV-eGFP reporter construct (FIG. 10; Charles River, Rockville, MD) for 6 h at 38.5° C. in a humidified 5% C02 incubator. Presumptive zygotes were then denuded and cultured for 7 days as already described. Embryos were imaged under a fluorescent microscope with a fluorescein isothiocyanate (FITC) filter throughout the culturing process to identify transduction efficiency.

Embryos were collected on day 7 and lysed in 10 μL of Epicenter DNA extraction buffer (Lucigen, Teddington, United Kingdom) by vortexing and using a thermal cycler at 65° C. for 6 min, 98° C. for 2 min and then held at 4° C. PCR was performed using primers aavGFPF2 and aavGFPR2 (Table 5) targeting the reporter construct developed using Primer Blast (NCBI) on a thermal cycler with 10 μL GoTaq Green Master Mix (Promega, Madison WI), 4.2 μL of water, 0.4 μL of each primer at 10 μM and 5 μL of DNA in lysis buffer for 5 min at 95° C., 35 cycles of 30 s at 95° C., 30 s at 60° C., and 30 s at 72° C., followed by 5 min at 72° C.

The second round of PCR was run using 10 μL GoTaq Green Master Mix, 4.2 μL of water, 0.4 μL of each primer at 10 μM and 5 μL of first round PCR with the same settings as the first round.

### 5. KI Cell Line Generation

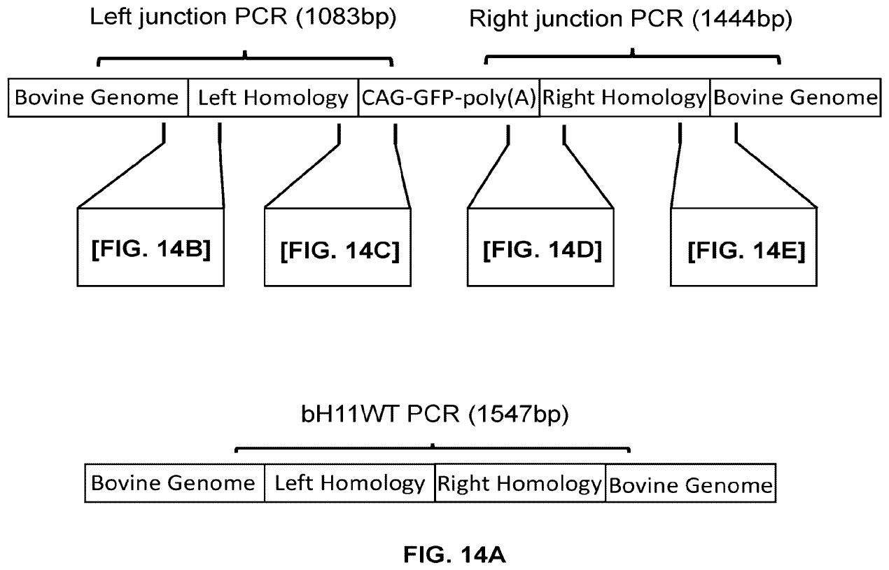

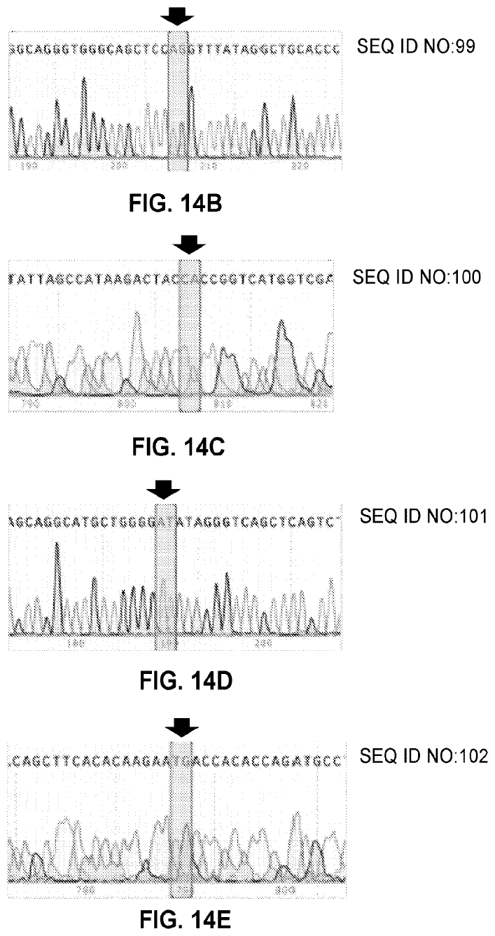

Bovine granulosa cells were aspirated along with COCs from follicles and cultured in mouse embryonic fibroblast (MEF) medium in 24-well plates at 38.5° C. in a humidified hypoxic atmosphere of 5% C02. Cells at 70% confluency were then lipofected with Lipofectamine 3000 and 500 ng of the rAAV6 plasmid containing the HDR donor template (FIG. 10). After 24 h, cell medium was changed and the cells were lipofected again using Lipofectamine CRISPRMAX with 125 ng of sgRNA, and 500 ng of Cas9 protein. Cells were then lysed using the Qiagen DNeasy blood and tissue kit according to manufacturer protocols and analyzed by PCR using primers flanking the 5′ (left) junction and 3′ (right) junction of the targeted insert (FIGS. 14A-14E).

6. rAAV6 Transduction with Matured COCs

Oocytes were collected and matured as already described. Groups of 25 matured COCs were transferred into 50 μL drops of SOF-IVF to be incubated with sperm and various concentrations of rAAV6 containing the HDR template for 6 h at 38.5° C. in a humidified 5% C02 incubator. Presumptive zygotes then immediately underwent electroporation as described previously.

7. rAAV6 Transduction with Denuded Oocytes

Oocytes were collected and matured as already described. Matured COCs were then denuded by vortex in SOF-HEPES for 5 min, and groups of 20 denuded oocytes and 5 COCs were transferred into 50 μL drops of SOF-IVF to be incubated with sperm and various concentrations of rAAV6 containing the HDR template for 6 h at 38.5° C. in a humidified 5% C02 incubator. Presumptive zygotes then immediately underwent electroporation as described previously.

### 8. Analysis of a Targeted Gene Sequence

Resulting blastocysts were analyzed under a fluorescent microscope with a FITC filter to identify GFP expression. Blastocysts were then collected and lysed in 10 μL of Epicenter DNA extraction buffer using a thermal cycler at 65° C. for 6 min, 98° C. for 2 min, and then held at 4° C. PCR primers were then designed using Primer Blast (NCBI) to target each gRNA cut site (Table 7).

TABLE 7
Sequence of primers used for PCR amplification of target region. For
indexes and barcodes, index and barcode sequences are underlined and
bolded.

| Primers | Sequence | SEQ ID NO: |
|---|---|---|
| H11F2 | CCCCAGTGTTGTGCATGTAG | 27 |
| H11R2 | GTGAATGCCACTGCTGTGTT | 28 |
| oH11F1 | CATGCTCAATCCACAAAGCCA | 37 |
| oH11R1 | TGTCTTCACCAAAAGGTGGC | 38 |
| ocH11F2 | TTGGACTGGGAGGAATGAAG | 39 |
| ocH11R2 | GGGCTGTTTCTTTTGGTTGA | 40 |
| bRosa26F1 | GGGAGGTGCATGTTCTCCAA | 41 |
| bRosa26R1 | TCTGTTTTGGCGGTGTAGCA | 42 |
| Bmpr2F1 | TGGCTCATGTGCTTAGTTGC | 43 |
| Bmpr2R1 | GAACAAGGGCCCTCAAGAAT | 44 |
| Bmpr2F2 | ACAGCAGAAGGACTTAGCCAT | 45 |
| Bmpr2R2 | TGCTTTGAGTTTGGAATTGCAC | 46 |
| bH11Fb | AGATCTCTCGAGGTTCCCCAGTGTTGTGCATGTAG | 47 |
| bH11Rb | GTAGTCGAATTCGTTGTGAATGCCACTGCTGTGTT | 48 |
| oH11Fb | AGATCTCTCGAGGTTATCGGAGCGGAGGAGTAAGA | 49 |
| oH11Rb | GTAGTCGAATTCGTTTGAAGCGAATGGCACTGTTG | 50 |
| BC1F | TCAGACGATGCGTCATAGATCTCTCGAGGTT | 51 |
| BC1R | TCAGACGATGCGTCATGTAGTCGAATTCGTT | 52 |
| BC17F | CATAGCGACTATCGTGAGATCTCTCGAGGTT | 53 |
| BC17R | CATAGCGACTATCGTGGTAGTCGAATTCGTT | 54 |
| BC29F | GCTCGACTGTGAGAGAAGATCTCTCGAGGTT | 55 |
| BC29R | GCTCGACTGTGAGAGAGTAGTCGAATTCGTT | 56 |
| BC34F | ACTCTCGCTCTGTAGAAGATCTCTCGAGGTT | 57 |
| BC34R | ACTCTCGCTCTGTAGAGTAGTCGAATTCGTT | 58 |
| BC38F | TGCTCGCAGTATCACAAGATCTCTCGAGGTT | 59 |
| BC38R | TGCTCGCAGTATCACAGTAGTCGAATTCGTT | 60 |
| BC40F | CAGTGAGAGCGCGATAAGATCTCTCGAGGTT | 61 |
| BC40R | CAGTGAGAGCGCGATAGTAGTCGAATTCGTT | 62 |
| BC48F | TCACACTCTAGAGCGAAGATCTCTCGAGGTT | 63 |
| BC48R | TCACACTCTAGAGCGAGTAGTCGAATTCGTT | 64 |
| BC52F | GCAGACTCTCACACGCAGATCTCTCGAGGTT | 65 |
| BC52R | GCAGACTCTCACACGCGTAGTCGAATTCGTT | 66 |
| BC54F | GTGTGAGATATATATCAGATCTCTCGAGGTT | 67 |
| BC54R | GTGTGAGATATATATCGTAGTCGAATTCGTT | 68 |
| BC62F | GACAGCATCTGCGCTCAGATCTCTCGAGGTT | 69 |
| BC62R | GACAGCATCTGCGCTCGTAGTCGAATTCGTT | 70 |
| BC70F | CTGCGCAGTACGTGCAAGATCTCTCGAGGTT | 71 |
| BC70R | CTGCGCAGTACGTGCAGTAGTCGAATTCGTT | 72 |
| BC9F | CTGCGTGCTCTACGACAGATCTCTCGAGGTT | 73 |
| BC9R | CTGCGTGCTCTACGACGTAGTCGAATTCGTT | 74 |
| bc1001F | CACATATCAGAGTGCGAGATCTCTCGAGGTT | 75 |
| bc1001R | CACATATCAGAGTGCGGTAGTCGAATTCGTT | 76 |
| Bc1002F | ACACACAGACTGTGAGAGATCTCTCGAGGTT | 77 |
| Bc1002R | ACACACAGACTGTGAGGTAGTCGAATTCGTT | 78 |
| Bc1003F | ACACATCTCGTGAGAGAGATCTCTCGAGGTT | 79 |
| Bc1003R | ACACATCTCGTGAGAGGTAGTCGAATTCGTT | 80 |
| Bc1004F | CACGCACACACGCGCGAGATCTCTCGAGGTT | 81 |
| Bc1004R | CACGCACACACGCGCGGTAGTCGAATTCGTT | 82 |
| Bc1006F | CATATATATCAGCTGTAGATCTCTCGAGGTT | 83 |
| Bc1006R | CATATATATCAGCTGTGTAGTCGAATTCGTT | 84 |
| Bc1002F | ACACACAGACTGTGAGAGATCTCTCGAGGTT | 85 |
| Bc1002R | ACACACAGACTGTGAGGTAGTCGAATTCGTT | 86 |
| Bc1007F | TCTGTATCTCTATGTGAGATCTCTCGAGGTT | 87 |
| Bc1007R | TCTGTATCTCTATGTGGTAGTCGAATTCGTT | 88 |
| Bc1008F | ACAGTCGAGCGCTGCGAGATCTCTCGAGGTT | 89 |
| Bc1008R | ACAGTCGAGCGCTGCGGTAGTCGAATTCGTT | 90 |
| Bc1009F | ACACACGCGAGACAGAAGATCTCTCGAGGTT | 91 |
| Bc1009R | ACACACGCGAGACAGAGTAGTCGAATTCGTT | 92 |
| Bc1010F | ACGCGCTATCTCAGAGAGATCTCTCGAGGTT | 93 |
| Bc1010R | ACGCGCTATCTCAGAGGTAGTCGAATTCGTT | 94 |

The target DNA region was amplified from individual blastocysts through two rounds of PCR. PCR was performed on a thermal cycler with 10 μL GoTaq GreenMaster Mix, 0.4 μL of each primer at 10 μM, and 9.2 μL of DNA in lysis buffer for 5 min at 95° C., 35 cycles of 30 s at 95° C., 30 s at 59° C., and 30 s at 72° C., followed by 5 min at 72° C. The second round of PCR was run with 10 μL GoTaq® Green Master Mix, 4.2 μL of water, 0.4 μL of each primer at 10 μM, and 5 μL of first round PCR using the same settings as the first round. PCR mixes for all species to evaluate the optimization of electroporation were the same. The bovine H11 and Rosa26loci were amplified using primers H11F2 and H11R2, or bRosa26F1 and bRosa26R1, respectively.

The ovine H11 and Bmpr2 loci were amplified using primers oH11F1 and oH11R1 in the first round and ocH11F2 and ocH11R2 in the second round, or Bmpr2F1 and Bmpr2R1 in the first round and Bmpr2F2 and Bmpr2R2 in the second round, respectively. For bovine blastocysts treated with rAAV6 and electroporation, the target region was amplified through two rounds of the PCR using primers flanking the 5′ (left) junction and 3′ (right) junction of the targeted insert (FIGS. 16A-16B, 15 and Table 7). PCR products were run on a 1% agarose gel with 5 μL of SYBR™ Safe (Thermo Fisher Scientific, Waltham, MA), visualized using a gel imager, purified using the QIAquick® Gel Extraction Kit (Qiagen, Hilden, Germany), and Sanger sequenced.

The blastocyst DNA was then analyzed for targeted mutations with TIDE (Ref 46) or integration of the donor template with DNA sequence alignment using SnapGene (Dotmatics, San Diego, CA).

### 9. Evaluation of Genetic Mosaicism

Electroporated bovine and ovine blastocysts were collected, lysed, and underwent whole-genome amplification using the Repli-G Mini kit (Qiagen, Inc., Valencia, CA). Whole-genome amplified samples were used for PCR amplification of cut sites using a dual round PCR approach already described to barcode each sample with a reduction from 35 to 5 cycles in the first round of PCR. Primers were designed using Primer3 to amplify each region with a 15 bp adapter sequence attached to the forward (AGATCTCTCGAGGTT; SEQ ID NO:35) and reverse (GTAGTCGAATTCGTT; SEQ ID NO:36), respectively (Table 7).

The second round of PCR amplified off the adapters adding an independent barcode for each sample to identify reads for pooled sequencing (Table 7). PCR samples underwent library preparation and were sequenced on an Illumina MiSeq600 sequencer by the UC Davis DNA Tech Core (Davis, CA). After we obtained sequencing reads from Illumina sequencer, we demultiplexed reads into individual samples according to barcode information using fastq-multx (github.com/brwnj/fastq-multx). The generated mate-pair reads were then merged using the FLASH tool (version 1.2.11) (Ref. 47).

Subsequently, merged reads were aligned against the respective reference genomes, Oar_rambouillet_v1.0 (Ensembl version 105) for sheep, and ARSUCD1.2 (Ensembl version 105) for cattle, using the “MEM” algorithm implemented in the BWA 0.7.17-r1188 software (Ref. 48). Then, CrispRVariants (Version 1.22.0) was employed to define, count, and localize variants for each sample. The called variants were then visualized using the “plotVariants” function of the CrispRVariants software package (Ref. 49).

### 10. Statistical Analysis

Mutation and blastocyst development outcomes for each electroporation parameter were analyzed using the generalized linear models logistic regression in R. Pairwise comparisons between different electroporation parameters were analyzed for statistical significance. A chi-square test with Yates correction was performed to examine the relationship between species and proportion of mosaic (>2 alleles) edited blastocysts.

### REFERENCES RELATED TO EXAMPLE 1

1. McFarlane G R, et al. On-farm livestock genome editing using cutting edge reproductive technologies. Front Sustain Food Syst 2019; 3; doi: 10.3389/fsufs.2019.00106
2. Keefer C L. Artificial cloning of domestic animals. Proc Natl Acad Sci USA 2015; 112(29):8874-8878; doi: 10.1073/pnas.1501718112
3. Lin J C, Van Eenennaam A L. Electroporation-mediated genome editing of livestock zygotes. Front Genet 2021; 12:648482.
4. Dumeau C E, et al. Introducing gene deletions by mouse zygote electroporation of Cas12a/Cpf1. Transgenic Res 2019; 28(5-6):525-535; doi: 10.1007/s11248-019-00168-9
5. Nakagawa Y, et al. Electroporation-mediated genome editing in vitrified/warmed mouse zygotes created by IVF via ultra-superovulation. Exp Anim 2018; 67(4):535-543; doi: 10.1538/expanim.18-0062
6. Peng H, et al. Efficient delivery of DNA and morpholinos into mouse preimplantation embryos by electroporation. PLos One 2012; 7(8); e43748; doi: 10.1371/journal.pone.0043748
7. Remy S, et al. Generation of gene-edited rats by delivery of CRISPR/Cas9 protein and donor DNA into intact zygotes using electroporation. Sci Rep 2017; 7; 16554; doi: 10.1038/s41598-017-16328-y
8. Teixeira M, et al. Electroporation of mice zygotes with dual guide RNA/Cas9 complexes for simple and efficient cloning-free genome editing. Sci Rep 2018; 8(1):474; doi: 10.1038/s41598-017-18826-5
9. Troder S E, et al. An optimized electroporation approach for efficient CRISPR/Cas9 genome editing in murine zygotes. PLoS One 2018; 13(5):e0196891; doi: 10.1371/journal.pone.0196891
10. Hirata M, et al. Genome mutation after introduction of the gene editing by electroporation of Cas9 protein (GEEP) system in matured oocytes and putative zygotes. In Vitro Cell Dev Biol Anim 2019; 55(4):237-242; doi: 10.1007/s11626-019-00338-3
11. Hirata M, et al. One-step genome editing of porcine zygotes through the electroporation of a CRISPR/Cas9 system with two guide RNAs. In Vitro Cell Dev Biol Anim 2020; 56(8):614-621.
12. Miao D, et al. Simplified pipelines for genetic engineering of mammalian embryos by CRISPR-Cas9 electroporationdagger. Biol Reprod 2019; 101(1):177-187; doi: 10.1093/biolre/ioz075
13. Tanihara F, et al. Generation of PDX-1 mutant porcine blastocysts by introducing CRISPR/Cas9-system into porcine zygotes via electroporation. Anim Sci J 2019; 90(1):55-61; doi: 10.1111/asj.13129
14. Tanihara F, et al. Efficient generation of GGTA1-deficient pigs by electroporation of the CRISPR/Cas9 system into in vitro-fertilized zygotes. BMC Biotechnol 2020; 20(1):40; doi: 10.1186/s12896-020-00638-7
15. Camargo L S A, et al. Efficient one-step knockout by electroporation of ribonucleoproteins into zona-intact bovine embryos. Front Genet 2020; 11(1047):570069; doi: 10.3389/fgene.2020.570069
16. Namula Z, et al. Genome mutation after the introduction of the gene editing by electroporation of Cas9 protein (GEEP) system into bovine putative zygotes. In Vitro Cell Dev Biol Anim 2019; 55(8):598-603; doi: 10.1007/s11626-019-00385-w
17. Tanihara F, et al. The effects of electroporation on viability and quality of in vivo-derived bovine blastocysts. J Reprod Dev 2019; 65(5):475-479; doi: 10.1262/jrd.2019-2049
18. Wei J, et al. Developing the laboratory conditions for bovine zygote-mediated genome editing by electroporation. In Proceedings of the World Congress on Genetics Applied to Livestock Production 2018 (Vol. 11:1118)
19. Mahdi A K, et al. Single-step genome editing of small ruminant embryos by electroporation. Int J Mol Sci 2022; 23(18):10218; doi: 10.3390/ijms231810218
20. Romeo C, et al. AAV diffuses across zona pellucida for effortless gene delivery to fertilized eggs. Biochem Biophys Res Commun 2020; 526(1):85-90; doi: 10.1016/j.bbrc.2020.03.026
21. Bagheri H, et al. TIE: A method to electroporate long DNA templates into preimplantation embryos for CRISPR-Cas9 gene editing. CRISPR J 2018; 1:223-229; doi: 10.1089/crispr.2017.0020
22. Chen S, et al. CRISPR-READI: Efficient generation of knock in mice by CRISPR RNP electroporation and AAV donor infection. Cell Rep 2019; 27(13):3780.e4-3789.e4; doi: 10.1016/j.celrep.2019.05.103
23. Nonnenmacher M, Weber T. Intracellular transport of recombinant adeno-associated virus vectors. Gene Ther 2012; 19(6):649-658; doi: 10.1038/gt.2012.6
24. Naso M F, et al. Adeno-associated virus (AAV) as a vector for gene therapy. BioDrugs 2017; 31(4):317-334; doi: 10.1007/s40259-017-0234-5
25. Galibert L, et al. Functional roles of the membrane associated AAV protein MAAP. Sci Rep 2021; 11(1):21698; doi: 10.1038/s41598-021-01220-7
26. Aponte-Ubillus J J, et al. Molecular design for recombinant adeno-associated virus (rAAV) vector production. Appl Microbiol Biotechnol 2018; 102(3):1045-1054; doi: 10.1007/s00253-017-8670-1
27. Hacker U T, et al. Towards clinical implementation of adeno-associated virus (AAV) vectors for cancer gene therapy: Current status and future perspectives. Cancers (Basel) 2020; 12(7):1889; doi:10.3390/cancers12071889
28. Mizuno N, et al. Intra-embryo gene cassette knockin by CRISPR/Cas9-mediated genome editing with adeno-associated viral vector. iScience 2018; 9:286-297; doi: 10.1016/j.isci.2018.10.030
29. Yoon Y, et al. Streamlined ex vivo and in vivo genome editing in mouse embryos using recombinant adeno-associated viruses. Nat Commun 2018; 9(1):412; doi: 10.1038/s41467-017-02706-7
30. Owen J R, et al. One-step generation of a targeted knock-in calf using the CRISPR-Cas9 system in bovine zygotes. BMC Genomics 2021; 22(1):118; doi: 10.1186/s12864-021-07418-3
31. Papapetrou E P, Schambach A. Gene insertion into genomic safe harbors for human gene therapy. Mol Ther 2016; 24(4):678-684; doi: 10.1038/mt.2016.38
32. Bishop T F, Van Eenennaam A L. Genome editing approaches to augment livestock breeding programs. J Exp Biol 2020; 223(Pt Suppl 1):jeb207159; doi: 10.1242/jeb.207159
33. Catala M G, et al. Brilliant Cresyl Blue stain selects largest oocytes with highest mitochondrial activity, maturation-promoting factor activity and embryo developmental competence in prepubertal sheep. Reproduction 2011; 142(4):517-527; doi: 10.1530/rep-10-0528
34. Chen W, et al. Massively parallel profiling and predictive modeling of the outcomes of CRISPR/Cas9-mediated double-strand break repair. Nucleic Acids Res 2019; 47(15):7989-8003; doi: 10.1093/nar/gkz487
35. Krivonogova A S, et al. AAV infection of bovine embryos: Novel, simple and effective tool for genome editing. Theriogenology 2022; 193(77-86); doi: 10.1016/j.theriogenology.2022.09.007
36. Zhang L, et al. Cumulus cell function during bovine oocyte maturation, fertilization, and embryo development in vitro. Mol Reprod Dev 1995; 40(3):338-344; doi: 10.1002/mrd.1080400310
37. Fatehi A N, et al. Effect of cumulus cell removal of in vitro matured bovine oocytes prior to in vitro fertilization on subsequent cleavage rate. Theriogenology 2002; 57(4):1347-1355; doi: 10.1016/S0093-691X(01)00717-8
38. Owen J R, et al. Harnessing endogenous repair mechanisms for targeted gene knock-in of bovine embryos. Sci Rep 2020; 10(1):16031; doi: 10.1038/s41598-020-72902-x
39. Lonergan P, et al. Factors influencing oocyte and embryo quality in cattle. Reprod Nutr Dev 2001; 41(5):427-437
40. Lamas-Toranzo I, et al. Strategies to reduce genetic mosaicism following CRISPR-mediated genome edition in bovine embryos. Sci Rep 2019; 9(1):14900; doi: 10.1038/s41598-019-51366-8
41. Salvesen H A, et al. Simulating the commercial implementation of gene-editing for influenza A virus resistance in pigs: An economic and genetic analysis. Genes 2022; 13(8):1436
42. Bogliotti Y S, et al. Efficient derivation of stable primed pluripotent embryonic stem cells from bovine blastocysts. Proc Natl Acad Sci USA 2018; 115(9):2090-2095; doi: 10.1073/pnas.1716161115
43. Hennig S L, et al. Evaluation of mutation rates, mosaicism and off target mutations when injecting Cas9 mRNA or protein for genome editing of bovine embryos. Sci Rep 2020; 10(1):22309; doi: 10.1038/s41598-020-78264-8
44. Yuan M, et al. HMEJ-based safe-harbor genome editing enables efficient generation of cattle with increased resistance to tuberculosis. J Biol Chem 2021; 296:100497; doi: 10.1016/j.jbc.2021.100497
45. Labun K, et al. CHOPCHOP v3: Expanding the CRISPR web toolbox beyond genome editing. Nucleic Acids Res 2019; 47(W1):W171-W174; doi: 10.1093/nar/gkz365
46. Brinkman E K, et al. Easy quantitative assessment of genome editing by sequence trace decomposition. Nucleic Acids Res 2014; 42(22):e168-e168; doi: 10.1093/nar/gku936
47. Magoĉ T, Salzberg S L. FLASH: Fast length adjustment of short reads to improve genome assemblies. Bioinformatics 2011; 27(21):2957-2963; doi: 10.1093/bioinformatics/btr507
48. Li H, Durbin R. Fast and accurate short read alignment with Burrows-Wheeler transform. Bioinformatics 2009; 25(14):1754-1760; doi: 10.1093/bioinformatics/btp324
49. Lindsay H, et al. CrispRVariants charts the mutation spectrum of genome engineering experiments. Nat Biotechnol 2016; 34(7):701-702; doi: 10.1038/nbt.3628

### Additional References of the Present Disclosure

Oikawa, M., et al., Functional primordial germ cell& #x2013; like cells from pluripotent stem cells in rats. Science, 2022. 376(6589): p. 176-179.
Vilanrino, M. et al., CRISPR/Cas9 microinjection in oocytes disables pancreas development in sheep. Scientific Reports, 2017. Vol. 7, Art. No. 17472, doi.org/10.1038/s41598-017-17805-0

### II. Sequences of the Present Disclosure

>HDR donor template (SEQ ID NO: 1):
CTATTACGCCAGCTGCGCGCTCGCTCGCTCACTGAGGCCGCCCGGGCAAAGCCCGG
GCGTCGGGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCGCCATGCT
ACTTATCTACGTAGCCATGCTCTAGGAAGATCGTAGGTTAATTAATCTAGACTGCAG
AGtagccataagactacctatAGGGGTTTATAGGCTGCACCCCTAACCCTTAGGCTGTTTTT
TTAACCTTTCAGTCAAAATCCAGTCATCTTGCTAACTTCACTTGTGCCGAACCA
GAATGGTCTCATTATTTGAGCCTCTACTATGAAGTCAAACTAGAGTTGAGGGCC
CTAAATGGATAAGAACTGAGAAATCGGCTGAGTTAAGTCGAGGAGCTTGAGAT
TTGTCACCTCAGATTGGGAGGAATAAAAAACCTTCAGTCAGGAAAGGGGACTC
AAGGCCATAGTTATTCTGGTAGAATCCTTTTCCCCAGTGTTGTGCATGTAGTTACGGT
ACACAGAATAACGGAACGGAGAAGTAAGAACACAGAAGAAGTTAACACAGGCACC
AGAGTCTTGAGGGAAGTTCTATATGGAAAAAATTCTGGAATGAATCAGAATACTAA
GGCTCCATTTTTCCCTATTGGGGACTCTGACTTGGAGACCCAGGAAGCCAACTGTTG
ACTTTTGCCCCAGTAAACGTGACAAAGGACCATATACCTGATTACCCAATAAATTAT
TTTCTCTAGTTGGGTTTAATTTTAGAAATTACACATTATCATCTGATATTAGCCATAA
GACTACCACCGGTCATGGTCGACATTGATTATTGACTAGTTATTAATAGTAATCAATT
ACGGGGTCATTAGTTCATAGCCCATATATGGAGTTCCGCGTTACATAACTTACGGTAA
ATGGCCCGCCTGGCTGACCGCCCAACGACCCCCGCCCATTGACGTCAATAATGACGTA
TGTTCCCATAGTAACGCCAATAGGGACTTTCCATTGACGTCAATGGGTGGAGTATTTA
CGGTAAACTGCCCACTTGGCAGTACATCAAGTGTATCATATGCCAAGTACGCCCCCTA
TTGACGTCAATGACGGTAAATGGCCCGCCTGGCATTATGCCCAGTACATGACCTTATG
GGACTTTCCTACTTGGCAGTACATCTACGTATTAGTCATCGCTATTACCATGGtcgaggtga
gccccacgttctgcttcactctccccatctcccccccctccccacccccaattttgtatttatttattttttaattattttgtgcagcgatgggggc
ggggggggggggggggcgcgcgccrggggggggggggggggggggggggggggggggggsgrggcggagaggtgcggcgg
cagccaatcagagcggcgcgctccgaaagtttccttttatggcgaggcggcggcggcggcggccctataaaaagcgaagcgcgcgg
cgggcgGGAGTCGCTGCGCGCTGCCTTCGCCCCGTGCCCCGCTCCGCCGCCGCCTCGCG
CCGCCCGCCCCGGCTCTGACTGACCGCGTTACTCCCACAGGTGAGCGGGCGGGACGG
CCCTTCTCCTCCGGGCTGTAATTAGCGCTTGGTTTAATGACGGCTTGTTTCTTTTCTGT
GGCTGCGTGAAAGCCTTGAGGGGCTCCGGGAGGGCCCTTTGTGCGGGGGGAGCGGC
TCGGGGGGTGCGTGCGTGTGTGTGTGCGTGGGGAGCGCCGCGTGCGGCTCCGCGCTG
CCCGGCGGCTGTGAGCGCTGCGGGCGCGGCGCGGGGCTTTGTGCGCTCCGCAGTGTG
CGCGAGGGGAGCGCGGCCGGGGGCGGTGCCCCGCGGTGCGGGGGGGGCTGCGAGG
GGAACAAAGGCTGCGTGCGGGGTGTGTGCGTGGGGGGGTGAGCAGGGGGTGTGGGC
GCGTCGGTCGGGCTGCAACCCCCCCTGCACCCCCCTCCCCGAGTTGCTGAGCACGGC
CCGGCTTCGGGTGCGGGGCTCCGTACGGGGCGTGGCGCGGGGCTCGCCGTGCCGGG
CGGGGGGTGGCGGCAGGTGGGGGTGCCGGGCGGGGGGGGGCCGCCKCGGGCCGGG
GRGGGCTCGGGGGRGGGGCGCGGCGGCCCCCGGAGCGCCGGCGGCTGTCGAGGCGC
GGCGAGCCGCAGCCATTGCCTTTTATGGTAATCGTGCGAGAGGGCGCAGGGACTTCC
TTTGTCCCAAATCTGTGCGGAGCCGAAATCTGGGAGGCGCCGCCGCACCCCCTCTAGC
GGGCGCGGGGCGAAGCGGTGCGGCGCCGGCAGGAAGGAAATGGGCGGGGAGGGCCT
TCGTGCGTCGCCGCGCCGCCGTCCCCTTCTCCCTCTCCAGCCTCGGGGCTGTCCGCGG
GGGGACGGCTGCCTTCGGGGGGGACGGGGCAGGGCGGGGTTCGGCTTCTGGCGTGT
GACCGGCGGCTCTAGAGCCTCTGCTAACCATGTTCATGCCTTCTTCTTTTTCCTACAGC
TCCTGGGCAACGTGCTGGTTATTGTGCTGTCTCATCATTTTGGCAAAGAATTGCTAGC
GCCGCCACCATGAGCAAAGGAGAAGAACTTTTCACTGGAGTTGTCCCAATTCTTGTTGAAT
TAGATGGTGATGTTAATGGGCACAAATTTTCTGTCCGTGGAGAGGGTGAAGGTGATGCTA
CAAACGGAAAACTCACCCTTAAATTTATTTGCACTACTGGAAAACTACCTGTTCCGTGGCCA
ACACTTGTCACTACTCTGACCTATGGTGTTCAATGCTTTTCCCGTTATCCGGATCACATGAA
ACGGCATGACTTTTTCAAGAGTGCCATGCCCGAAGGTTATGTACAGGAACGCACTATATCT
TTCAAAGATGACGGGACCTACAAGACGCGTGCTGAAGTCAAGTTTGAAGGTGATACCCTT
GTTAATCGTATCGAGTTAAAGGGTATTGATTTTAAAGAAGATGGAAACATTCTTGGACACAA
ACTCGAGTACAACTTTAACTCACACAATGTATACATCACGGCAGACAAACAAAAGAATGGA
ATCAAAGCTAACTTCAAAATTCGCCACAACGTTGAAGATGGTTCCGTTCAACTAGCAGACC
ATTATCAACAAAATACTCCAATTGGCGATGGCCCTGTCCTTTTACCAGACAACCATTACCTG
TCGACACAATCTGTCCTTTCGAAAGATCCCAACGAAAAGCGTGACCACATGGTCCTTCTTG
AGTTTGTAACTGCTGCTGGGATTACACATGGCATGGATGAGCTCTACAAGAAAAGGCCGG
CGGCCACGAAAAAGGCCGGCCAGGCAAAAAAGAAAAAGTGAGAATTCTAACTAGAGCT
CGCTGATCAGCCTCGACTGTGCCTTCTAGTTGCCAGCCATCTGTTGTTTGCCCCT
CCCCCGTGCCTTCCTTGACCCTGGAAGGTGCCACTCCCACTGTCCTTTCCTAAT
AAAATGAGGAAATTGCATCGCATTGTCTGAGTAGGTGTCATTCTATTCTGGGGG
GTGGGGGGGGCAGGACAGCAAGGGGGAGGATTGGGAAGAGAATAGCAGGCA
TGCTGGGGAtatagggtcagctcagtctaaactcacccattggagtcattaggctcaagaaagagggccatggettcctcc
tcccagtcagagttcaggtccatgagtcctacagaaaaaactcaaggtaatttccctcaagaacctactgaagaggcagggattca
ggaagaagaaaacacagcagtggcattcacttcattcacacacttagcatatgcaactttaatcaaccaaaagaaaaatccccaa
ctgtacaagtgaaaaaaaatccaaaatgttgacacaggcttacctaacaccagctacttttatgtacagctgtataatttcaaaaaa
gctatcctatatgtatgtacaaaagttgtttatacacaggtctgtacataagggtctatacatttatttcctcagaacccttaaggtgcc
acctcttggtgaagacaccaacacttcattcacatatcttacaaaaaaagactttctaggactgacaatactgcatcctgtattcaaa
ccatgtagactccactgactggttaagttccttccatcataacgcagaccacaccagaagcctgggtgccctagacagcttcacaca
agaattagccataagactacctatAGGGCATTCATTTTATGTTTCAGGTTCAGGGGGAGATGTGG
GAGGTTTTTTAAAGCAAGTAAAACCTCTACAAATGTGGTAAAATCGATAAGGATCTT
CCTAGAGCATGGCTACGTAGATAAGTAGCATGGCGGGTTAATCATTAACTACAaggaa
cccctagtgatggagttggccactccctctctgcgcgctcgctcgctcactgaggccgggcgaccaaaggtcgcccgacgcccgggcttt
gcccgggcggcctcagtgagcgagcgagegcgcagCTGCATTAATGAATCGGCCAACGCGCGGGGAGA
GGCGGTTTGCGTATTGGGCGCTCTTCCGCTTCCTCGCTCACTGACTCGCTGCGCTCGG
TCGTTCGGCTGCGGCGAGCGGTATCAGCTCACTCAAAGGCGGTAATACGGTTATCCA
CAGAATCAGGGGATAACGCAGGAAAGAACATGTGAGCAAAAGGCCAGCAAAAGGC

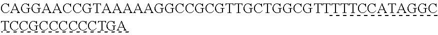

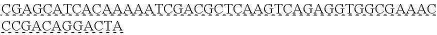

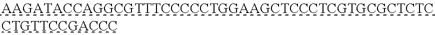

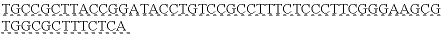

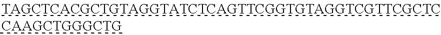

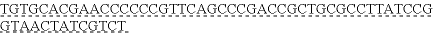

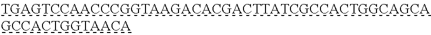

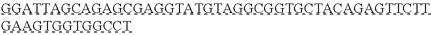

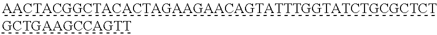

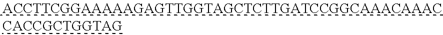

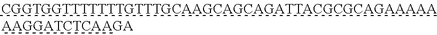

AGATCCTTTGATCTTTTCTACGGGGTCTGACGCTCAGTGGAACGAAAACTCACGTTA
AGGGATTTTGGTCATGAGATTATCAAAAAGGATCTTCACCTAGATCCTTTTAAATTA

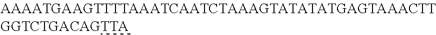

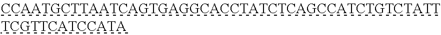

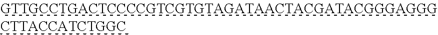

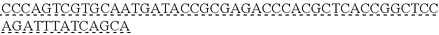

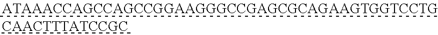

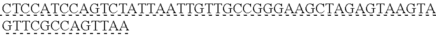

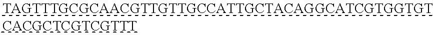

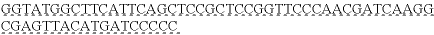

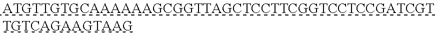

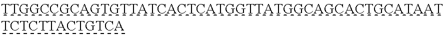

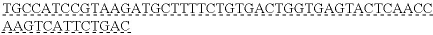

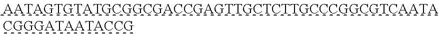

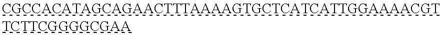

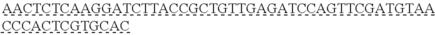

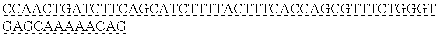

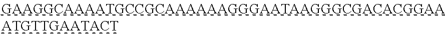

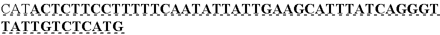

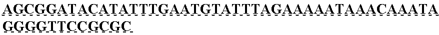

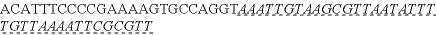

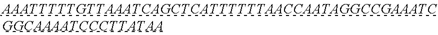

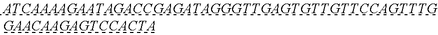

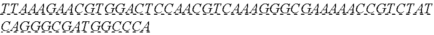

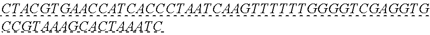

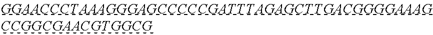

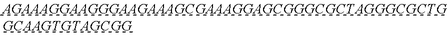

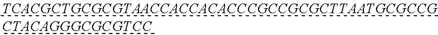

CATTCGCCATTCAGGCTGCGCAACTGTTGGGAAGGGCGATCGGTGCGGGCCTCTTCG
Legend for elements in HDR donor template (SEQ ID NO: 1)

| | H11 gRNA recognition | | |
|---|---|---|---|
| AAV2 ITR | site (lower case) | Left homology region | CMV enhancer |
| Chicken beta actin promoter | Chimeric intron | Superfolder GFP | Nuclear localization |
| (lower case) | | | signal |
| bGH poly(A) signal | right homology region | H11 gRNA | AAV2 ITS (lower case) |
| | (lower case) | recognition site (lower | |
| | | case) | |
| 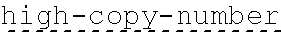 | 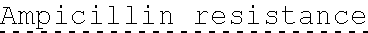 | 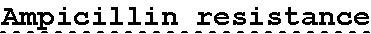 | 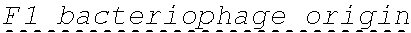 |
| 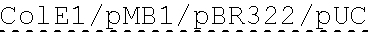 | | 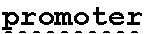 | 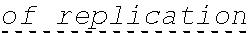 |
| 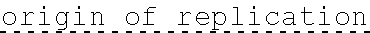 | | | |

>nuclear export signal (SEQ ID NO: 2)
LXXXLXXLXL
>endoplasmic reticulum localizaton/retention signal (SEQ ID NO: 3)
KDEL
>glycine linker (GGGGG)3 (SEQ ID NO: 4)
GGGGG
>glycine serine linkers (GGGGS)3 (SEQ ID NO: 5).
GGGGS
>NLS of the SV40 virus large T-antigen (SEQ ID NO: 6)
PKKKRKV
>another NLS of the SV40 virus large T-antigen (SEQ ID NO: 7)
PKKKRKVEAS
>the NLS from nucleoplasmin (SEQ ID NO: 8)
KRPAATKKAGQAKKKK
>a c-myc NLS (SEQ ID NO: 9)
PAAKRVKLD
>another c-myc NLS (SEQ ID NO: 10)
RQRRNELKRSP
>hRNPA1 M9 NLS (SEQ ID NO: 11)
NQSSNFGPMKGGNFGGRSSGPYGGGGQYFAKPRNQGGY
>IBB domain from importin-alpha (SEQ ID NO: 12)
RMRIZFKNKGKDTAELRRRRVEVSVELRKAKKDEQILKRRNV
>NLS of the myoma T protein (SEQ ID NO: 13)
VSRKRPRP
>another NLS of the myoma T protein (SEQ ID NO: 14)
PPKKARED
>NLS of the human p53 sequence (SEQ ID NO: 15)
PQPKKKPL
>NLS of mouse c-abl IV (SEQ ID NO: 16)
SALIKKKKKMAP
>NLS of influenza virus NS1 (SEQ ID NO: 17)
DRLRR
>another NLS of influenza virus NS1 (SEQ ID NO: 18)
PKQKKRK
>NLS of Hepatitis virus delta antigen (SEQ ID NO: 19)
RKLKKKIKKL
>NLS of mouse Mx1 protein (SEQ ID NO: 20)
REKKKFLKRR
>NLS of human poly(ADP-ribose) polymerase (SEQ ID NO: 21)
KRKGDEVDGVDEVAKKKSKK
>NLS of steroid hormone receptors (human) glucocorticoid (SEQ ID NO: 22)
RKCLQAGMNLEARKTKK
>H11 bH11LjuncF2 primer (la) (SEQ ID NO: 23)
TGCCACTGTTGCTTGAGACT
>H11 bH11LjuncR2 primer (1b) (SEQ ID NO: 24)
CCAAGTGGGCAGTTTACCGT
>H11 bH11RjuncF2 primer (2a) (SEQ ID NO: 25)
TGCTGGGATTACACATGGCA
>H11 bH11RjuncR2 primer (2b) (SEQ ID NO: 26)
AAGCACGGCCTAGTGGAGAA
>H11 H11F2 primer (SEQ ID NO: 27)
CCCCAGTGTTGTGCATGTAG
>H11 H11R2 primer (SEQ ID NO: 28)
GTGAATGCCACTGCTGTGTT
>Donor Template GFP insert aavGFPF (SEQ ID NO: 29)
ATGGTAATCGTGCGAGAGGG
>Donor Template GFP insert aavGFPR (SEQ ID NO: 30)
GGCCACGGAACAGGTAGTTT
>gRNA targeting the bovine H11 locus (SEQ ID NO: 31)
TAGCCATAAGACTACCTAT
>gRNA targeting Rosa26 locus in the bovine genome (SEQ ID NO: 32)
TGTCGAGTCTCGATTATGGG
>gRNA targeting the H1l locus in the ovine genome (SEQ ID NO: 33)
TAGCCACAAGACTACCTAT
>gRNA targeting Bmpr2 (SEQ ID NO: 34)
CAATTCAGAATGGAACGTAC
>forward 15 bp adapter sequence (SEQ ID NO: 35)
AGATCTCTCGAGGTT
>reverse 15 bp adapter sequence (SEQ ID NO: 36)
GTAGTCGAATTCGTT
>oH11F1 primer (SEQ ID NO: 37)
CATGCTCAATCCACAAAGCCA
>oH11R1 primer (SEQ ID NO: 38)
TGTCTTCACCAAAAGGTGGC
>ocH11F2 primer (SEQ ID NO: 39)
TTGGACTGGGAGGAATGAAG
>ocH11R2 primer (SEQ ID NO: 40)
GGGCTGTTTCTTTTGGTTGA
>bRosa26F1 primer (SEQ ID NO: 41)
GGGAGGTGCATGTTCTCCAA
>bRosa26R1 primer (SEQ ID NO: 42)
TCTGTTTTGGCGGTGTAGCA
>Bmpr2F1 primer (SEQ ID NO: 43)
TGGCTCATGTGCTTAGTTGC
>Bmpr2R1 primer (SEQ ID NO: 44)
GAACAAGGGCCCTCAAGAAT
>Bmpr2F2 primer (SEQ ID NO: 45)
ACAGCAGAAGGACTTAGCCAT
>Bmpr2R2 primer (SEQ ID NO: 46)
TGCTTTGAGTTTGGAATTGCAC
>bH11Fb primer (SEQ ID NO: 47)
AGATCTCTCGAGGTTCCCCAGTGTTGTGCATGTAG
>bH11Rb primer (SEQ ID NO: 48)
GTAGTCGAATTCGTTGTGAATGCCACTGCTGTGTT
>0H11Fb primer (SEQ ID NO: 49)
AGATCTCTCGAGGTTATCGGAGCGGAGGAGTAAGA
>oH11Rb primer (SEQ ID NO: 50)
GTAGTCGAATTCGTTTGAAGCGAATGGCACTGTTG
>BCIF primer (SEQ ID NO: 51)
TCAGACGATGCGTCATAGATCTCTCGAGGTT
>BC1R primer (SEQ ID NO: 52)
TCAGACGATGCGTCATGTAGTCGAATTCGTT
>BC17F primer (SEQ ID NO: 53)
CATAGCGACTATCGTGAGATCTCTCGAGGTT
>BC17R primer (SEQ ID NO: 54)
CATAGCGACTATCGTGGTAGTCGAATTCGTT
>BC29F primer (SEQ ID NO: 55)
GCTCGACTGTGAGAGAAGATCTCTCGAGGTT
>BC29R primer (SEQ ID NO: 56)
GCTCGACTGTGAGAGAGTAGTCGAATTCGTT
>BC34F primer (SEQ ID NO: 57)
ACTCTCGCTCTGTAGAAGATCTCTCGAGGTT
>BC34R primer (SEQ ID NO: 58)
ACTCTCGCTCTGTAGAGTAGTCGAATTCGTT
>BC38F primer (SEQ ID NO: 59)
TGCTCGCAGTATCACAAGATCTCTCGAGGTT
>BC38R primer (SEQ ID NO: 60)
TGCTCGCAGTATCACAGTAGTCGAATTCGTT
>BC40F primer (SEQ ID NO: 61)
CAGTGAGAGCGCGATAAGATCTCTCGAGGTT
>BC40R primer (SEQ ID NO: 62)
CAGTGAGAGCGCGATAGTAGTCGAATTCGTT
>BC48F primer (SEQ ID NO: 63)
TCACACTCTAGAGCGAAGATCTCTCGAGGTT
>BC48R primer (SEQ ID NO: 64)
TCACACTCTAGAGCGAGTAGTCGAATTCGTT
>BC52F primer (SEQ ID NO: 65)
GCAGACTCTCACACGCAGATCTCTCGAGGTT
>BC52R primer (SEQ ID NO: 66)
GCAGACTCTCACACGCGTAGTCGAATTCGTT
>BC54F primer (SEQ ID NO: 67)
GTGTGAGATATATATCAGATCTCTCGAGGTT
>BC54R primer (SEQ ID NO: 68)
GTGTGAGATATATATCGTAGTCGAATTCGTT
>BC62F primer (SEQ ID NO: 69)
GACAGCATCTGCGCTCAGATCTCTCGAGGTT
>BC62R primer (SEQ ID NO: 70)
GACAGCATCTGCGCTCGTAGTCGAATTCGTT
>BC7OF primer (SEQ ID NO: 71)
CTGCGCAGTACGTGCAAGATCTCTCGAGGTT
>BC70R primer (SEQ ID NO: 72)
CTGCGCAGTACGTGCAGTAGTCGAATTCGTT
>BC9F primer (SEQ ID NO: 73)
CTGCGTGCTCTACGACAGATCTCTCGAGGTT
>BC9R primer (SEQ ID NO: 74)
CTGCGTGCTCTACGACGTAGTCGAATTCGTT
>bc1001F primer (SEQ ID NO: 75)
CACATATCAGAGTGCGAGATCTCTCGAGGTT
>bc1001R primer (SEQ ID NO: 76)
CACATATCAGAGTGCGGTAGTCGAATTCGTT
>Bc1002F primer (SEQ ID NO: 77)
ACACACAGACTGTGAGAGATCTCTCGAGGTT
>Bc1002R primer (SEQ ID NO: 78)
ACACACAGACTGTGAGGTAGTCGAATTCGTT
>Bc1003F primer (SEQ ID NO: 79)
ACACATCTCGTGAGAGAGATCTCTCGAGGTT
>Bc1003R primer (SEQ ID NO: 80)
ACACATCTCGTGAGAGGTAGTCGAATTCGTT
>Bc1004F primer (SEQ ID NO: 81)
CACGCACACACGCGCGAGATCTCTCGAGGTT
>Bc1004R primer (SEQ ID NO: 82)
CACGCACACACGCGCGGTAGTCGAATTCGTT
>Bc1006F primer (SEQ ID NO: 83)
CATATATATCAGCTGTAGATCTCTCGAGGTT
>Bc1006R primer (SEQ ID NO: 84)
CATATATATCAGCTGTGTAGTCGAATTCGTT
>Bc1002F primer (SEQ ID NO: 85)
ACACACAGACTGTGAGAGATCTCTCGAGGTT
>Bc1002R primer (SEQ ID NO: 86)
ACACACAGACTGTGAGGTAGTCGAATTCGTT
>Bc1007F primer (SEQ ID NO: 87)
TCTGTATCTCTATGTGAGATCTCTCGAGGTT
>Bc1007R primer (SEQ ID NO: 88)
TCTGTATCTCTATGTGGTAGTCGAATTCGTT
>Bc1008F primer (SEQ ID NO: 89)
ACAGTCGAGCGCTGCGAGATCTCTCGAGGTT
>Bc1008R primer (SEQ ID NO: 90)
ACAGTCGAGCGCTGCGGTAGTCGAATTCGTT
>Bc1009F primer (SEQ ID NO: 91)
ACACACGCGAGACAGAAGATCTCTCGAGGTT
>Bc1009R primer (SEQ ID NO: 92)
ACACACGCGAGACAGAGTAGTCGAATTCGTT
>Bc1010F primer (SEQ ID NO: 93)
ACGCGCTATCTCAGAGAGATCTCTCGAGGTT
>Bc1010R primer (SEQ ID NO: 94)
ACGCGCTATCTCAGAGGTAGTCGAATTCGTT
>bH11WTF2 primer (SEQ ID NO: 95)
AGGCAGACCTCATGCTCAAT
>bH11WTR2 primer (SEQ ID NO: 96)
CTCCATGCCCACCAAAGTCA
>aavGFPF2 primer (SEQ ID NO: 97)
GCAAGCTGACCCTGAAGTTC
>aavGFPR2 primer (SEQ ID NO: 98)
CTTCTCGTTGGGGTCTTTGC
>FIG. 14B sequence (SEQ ID NO: 99)
GGCAGGGTGGGCAGCTCCAGGTTTATAGGCTGCACCC
>FIG. 14C sequence (SEQ ID NO: 100)
TATTAGCCATAAGACTACCACCGGTCATGGTCGA
>FIG. 14D sequence (SEQ ID NO: 101)
AGCAGGCATGCTGGGGATATAGGGTCAGCTCAGTC
>FIG. 14E sequence (SEQ ID NO: 102)
CAGCTTCACACAAGAATGACCACACCAGATGCC

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Various modifications and variations of the described methods, pharmaceutical compositions, and kits of the present disclosure will be apparent to those skilled in the art without departing from the scope and spirit of the present disclosure. Although the present disclosure has been described in connection with specific embodiments, it will be understood that it is capable of further modifications and that the present disclosure as claimed (i.e., combination of the features of the claims of the present disclosure) should not be unduly limited to such specific embodiments. Indeed, various modifications of the described modes for carrying out the present disclosure that are obvious to those skilled in the art are intended to be within the scope of the present disclosure. This application is intended to cover any variations, uses, or adaptations of the present disclosure following, in general, the principles of the present disclosure and including such departures from the present disclosure come within known customary practice within the art to which the present disclosure pertains and may be applied to the essential features herein before set forth.

1. A method of producing a fertilized embryo comprising a genomic edit, the method comprising:
a) providing isolated mature cumulus-oocyte complexes (COC) from a first non-human organism;
b) removing the cumulus cells from the isolated mature COC to produce denuded oocytes;
c) incubating the denuded oocytes with sperm, adeno-associated virus (AAV) particles comprising a donor nucleic acid having homology to a nucleotide sequence adjacent to a target genomic site, and supplemental mature COC from a second non-human organism to produce a first plurality of cells comprising at least one zygote comprising the donor nucleic acid;
d) removing the cumulus cells from the first plurality of cells thereby producing a plurality of denuded cells comprising at least one denuded zygote;
e) electroporating the plurality of denuded cells in the presence of a site-directed nuclease that binds to the target genomic site; and
f) culturing the electroporated plurality of denuded cells to produce at least one fertilized embryo comprising the genomic edit.

2. The method of claim 1, wherein the AAV particles are AAV1, AAV2, AAV3, AAV4, AAV5, AAV6, AAV7, AAV8, or AAV9 particles.

3. The method of claim 1, wherein the AAV particles are AAV6 particles.

4. The method of claim 1, wherein the AAV particles are recombinant AAV particles.

5. The method of claim 1, wherein the donor nucleic acid is a portion of a donor template.

6. The method of claim 5, wherein the donor template is part of a plasmid or linear nucleic acid.

7. The method of claim 1, wherein step c) comprises incubating the denuded oocytes and the supplemental mature COC at an approximate ratio of 4:1.

8. The method of claim 1, wherein the incubating of step c) is performed for 6 hours.

9. The method of claim 1, wherein the site-directed nuclease is a Cas protein.

10. The method of claim 9, wherein the Cas protein is selected from the group consisting of Cas5, Cas6, Cas7, Cas8, Cas9, Cas12a, Cas12b, Cas12i, Cas12j, Cas12L, Cas12e, Cas12c, Cas12d, Cas12g, Cas12h, TnpB, Cas13a, Cas13b, Cas14, and nickase or deactivated versions thereof.

11. The method of claim 9, wherein the Cas protein is a Cas9 enzyme.

12. The method of claim 9, wherein the Cas protein is a Cas12a enzyme.

13. The method of claim 9, wherein the Cas protein comprises a nuclear localization signal.

14. The method of claim 9, wherein the Cas protein is part of a ribonucleoprotein (RNP), the RNP comprising the Cas protein bound to a guide RNA comprising a nucleotide sequence having complementarity to a binding site at the target genomic site.

15. The method of claim 1, wherein the genomic edit is an excision, an insertion, or a replacement of at least a portion of the target genomic site.

16. The method of claim 1, wherein the genomic edit is a gene knock-in at the target genomic site.

17. The method of claim 1, wherein the first non-human organism and the second non-human organism are the same organism.

18. The method of claim 1, wherein the first non-human organism and the second non-human organism are different organisms.

19. A fertilized embryo comprising the genomic edit produced by the method of claim 1.

20. A non-human organism comprising the genomic edit developed from the at least one fertilized embryo of claim 1.

21. A method of producing a non-human organism comprising the genomic edit comprising implanting the at least one fertilized embryo of claim 1 into a female non-human organism for gestation.


SEQUENCE LISTING (102 sequences)

SEQ ID NO: 1 - DNA, 7014 bp, synthetic construct
  source 1..7014: mol_type = other DNA
ctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcgtaggttaattaatctagactgcagagtagccataagactacctataggggtttataggctgcacccctaacccttaggctgtttttttaacctttcagtcaaaatccagtcatcttgctaacttcacttgtgccgaaccagaatggtctcattatttgagcctctactatgaagtcaaactagagttgagggccctaaatggataagaactgagaaatcggctgagttaagtcgaggagcttgagatttgtcacctcagattgggaggaataaaaaaccttcagtcaggaaaggggactcaaggccatagttattctggtagaatccttttccccagtgttgtgcatgtagttacggtacacagaataacggaacggagaagtaagaacacagaagaagttaacacaggcaccagagtcttgagggaagttctatatggaaaaaattctggaatgaatcagaatactaaggctccatttttccctattggggactctgacttggagacccaggaagccaactgttgacttttgccccagtaaacgtgacaaaggaccatatacctgattacccaataaattattttctctagttgggtttaattttagaaattacacattatcatctgatattagccataagactaccaccggtcatggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccrggggggggggggggggggggggggggggggggggsgrggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcckcgggccggggrgggctcgggggrggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctagcgccgccaccatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaagaaaaggccggcggccacgaaaaaggccggccaggcaaaaaagaaaaagtgagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggatatagggtcagctcagtctaaactcacccattggagtcattaggctcaagaaagagggccatggcttcctcctcccagtcagagttcaggtccatgagtcctacagaaaaaactcaaggtaatttccctcaagaacctactgaagaggcagggattcaggaagaagaaaacacagcagtggcattcacttcattcacacacttagcatatgcaactttaatcaaccaaaagaaaaatccccaactgtacaagtgaaaaaaaatccaaaatgttgacacaggcttacctaacaccagctacttttatgtacagctgtataatttcaaaaaagctatcctatatgtatgtacaaaagttgtttatacacaggtctgtacataagggtctatacatttatttcctcagaacccttaaggtgccacctcttggtgaagacaccaacacttcattcacatatcttacaaaaaaagactttctaggactgacaatactgcatcctgtattcaaaccatgtagactccactgactggttaagttccttccatcataacgcagaccacaccagaagcctgggtgccctagacagcttcacacaagaattagccataagactacctatagggcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcg

SEQ ID NO: 2 - protein, 10 aa, synthetic construct
  source 1..10: note = X = any one of A, R, N, D, C, Q, E, G, H, I, L, K, M, F, P, O, S, U, T, W, Y, or V
LXXXLXXLXL

SEQ ID NO: 3 - protein, 4 aa, synthetic construct
KDEL

SEQ ID NO: 4 - protein, 5 aa, synthetic construct
GGGGG

SEQ ID NO: 5 - protein, 5 aa, synthetic construct
GGGGS

SEQ ID NO: 6 - protein, 7 aa, synthetic construct
PKKKRKV

SEQ ID NO: 7 - protein, 10 aa, synthetic construct
PKKKRKVEAS

SEQ ID NO: 8 - protein, 16 aa, synthetic construct
KRPAATKKAGQAKKKK

SEQ ID NO: 9 - protein, 9 aa, synthetic construct
PAAKRVKLD

SEQ ID NO: 10 - protein, 11 aa, synthetic construct
RQRRNELKRSP

SEQ ID NO: 11 - protein, 38 aa, synthetic construct
NQSSNFGPMKGGNFGGRSSGPYGGGGQYFAKPRNQGGY

SEQ ID NO: 12 - protein, 42 aa, synthetic construct
RMRIZFKNKGKDTAELRRRRVEVSVELRKAKKDEQILKRRNV

SEQ ID NO: 13 - protein, 8 aa, synthetic construct
VSRKRPRP

SEQ ID NO: 14 - protein, 8 aa, synthetic construct
PPKKARED

SEQ ID NO: 15 - protein, 8 aa, synthetic construct
PQPKKKPL

SEQ ID NO: 16 - protein, 12 aa, synthetic construct
SALIKKKKKMAP

SEQ ID NO: 17 - protein, 5 aa, synthetic construct
DRLRR

SEQ ID NO: 18 - protein, 7 aa, synthetic construct
PKQKKRK

SEQ ID NO: 19 - protein, 10 aa, synthetic construct
RKLKKKIKKL

SEQ ID NO: 20 - protein, 10 aa, synthetic construct
REKKKFLKRR

SEQ ID NO: 21 - protein, 20 aa, synthetic construct
KRKGDEVDGVDEVAKKKSKK

SEQ ID NO: 22 - protein, 17 aa, synthetic construct
RKCLQAGMNLEARKTKK

SEQ ID NO: 23 - DNA, 20 bp, synthetic construct
  source 1..20: mol_type = other DNA
tgccactgttgcttgagact

SEQ ID NO: 24 - DNA, 20 bp, synthetic construct
  source 1..20: mol_type = other DNA
ccaagtgggcagtttaccgt

SEQ ID NO: 25 - DNA, 20 bp, synthetic construct
  source 1..20: mol_type = other DNA
tgctgggattacacatggca

SEQ ID NO: 26 - DNA, 20 bp, synthetic construct
  source 1..20: mol_type = other DNA
aagcacggcctagtggagaa

SEQ ID NO: 27 - DNA, 20 bp, synthetic construct
  source 1..20: mol_type = other DNA
ccccagtgttgtgcatgtag

SEQ ID NO: 28 - DNA, 20 bp, synthetic construct
  source 1..20: mol_type = other DNA
gtgaatgccactgctgtgtt

SEQ ID NO: 29 - DNA, 20 bp, synthetic construct
  source 1..20: mol_type = other DNA
atggtaatcgtgcgagaggg

SEQ ID NO: 30 - DNA, 20 bp, synthetic construct
  source 1..20: mol_type = other DNA
ggccacggaacaggtagttt

SEQ ID NO: 31 - RNA, 19 nt, synthetic construct
  source 1..19: mol_type = other RNA
tagccataagactacctat

SEQ ID NO: 32 - RNA, 20 nt, synthetic construct
  source 1..20: mol_type = other RNA
tgtcgagtctcgattatggg

SEQ ID NO: 33 - RNA, 19 nt, synthetic construct
  source 1..19: mol_type = other RNA
tagccacaagactacctat

SEQ ID NO: 34 - RNA, 20 nt, synthetic construct
  source 1..20: mol_type = other RNA
caattcagaatggaacgtac

SEQ ID NO: 35 - DNA, 15 bp, synthetic construct
  source 1..15: mol_type = other DNA
agatctctcgaggtt

SEQ ID NO: 36 - DNA, 15 bp, synthetic construct
  source 1..15: mol_type = other DNA
gtagtcgaattcgtt

SEQ ID NO: 37 - DNA, 21 bp, synthetic construct
  source 1..21: mol_type = other DNA
catgctcaatccacaaagcca

SEQ ID NO: 38 - DNA, 20 bp, synthetic construct
  source 1..20: mol_type = other DNA
tgtcttcaccaaaaggtggc

SEQ ID NO: 39 - DNA, 20 bp, synthetic construct
  source 1..20: mol_type = other DNA
ttggactgggaggaatgaag

SEQ ID NO: 40 - DNA, 20 bp, synthetic construct
  source 1..20: mol_type = other DNA
gggctgtttcttttggttga

SEQ ID NO: 41 - DNA, 20 bp, synthetic construct
  source 1..20: mol_type = other DNA
gggaggtgcatgttctccaa

SEQ ID NO: 42 - DNA, 20 bp, synthetic construct
  source 1..20: mol_type = other DNA
tctgttttggcggtgtagca

SEQ ID NO: 43 - DNA, 20 bp, synthetic construct
  source 1..20: mol_type = other DNA
tggctcatgtgcttagttgc

SEQ ID NO: 44 - DNA, 20 bp, synthetic construct
  source 1..20: mol_type = other DNA
gaacaagggccctcaagaat

SEQ ID NO: 45 - DNA, 21 bp, synthetic construct
  source 1..21: mol_type = other DNA
acagcagaaggacttagccat

SEQ ID NO: 46 - DNA, 22 bp, synthetic construct
  source 1..22: mol_type = other DNA
tgctttgagtttggaattgcac

SEQ ID NO: 47 - DNA, 35 bp, synthetic construct
  source 1..35: mol_type = other DNA
agatctctcgaggttccccagtgttgtgcatgtag

SEQ ID NO: 48 - DNA, 35 bp, synthetic construct
  source 1..35: mol_type = other DNA
gtagtcgaattcgttgtgaatgccactgctgtgtt

SEQ ID NO: 49 - DNA, 35 bp, synthetic construct
  source 1..35: mol_type = other DNA
agatctctcgaggttatcggagcggaggagtaaga

SEQ ID NO: 50 - DNA, 35 bp, synthetic construct
  source 1..35: mol_type = other DNA
gtagtcgaattcgtttgaagcgaatggcactgttg

SEQ ID NO: 51 - DNA, 31 bp, synthetic construct
  source 1..31: mol_type = other DNA
tcagacgatgcgtcatagatctctcgaggtt

SEQ ID NO: 52 - DNA, 31 bp, synthetic construct
  source 1..31: mol_type = other DNA
tcagacgatgcgtcatgtagtcgaattcgtt

SEQ ID NO: 53 - DNA, 31 bp, synthetic construct
  source 1..31: mol_type = other DNA
catagcgactatcgtgagatctctcgaggtt

SEQ ID NO: 54 - DNA, 31 bp, synthetic construct
  source 1..31: mol_type = other DNA
catagcgactatcgtggtagtcgaattcgtt

SEQ ID NO: 55 - DNA, 31 bp, synthetic construct
  source 1..31: mol_type = other DNA
gctcgactgtgagagaagatctctcgaggtt

SEQ ID NO: 56 - DNA, 31 bp, synthetic construct
  source 1..31: mol_type = other DNA
gctcgactgtgagagagtagtcgaattcgtt

SEQ ID NO: 57 - DNA, 31 bp, synthetic construct
  source 1..31: mol_type = other DNA
actctcgctctgtagaagatctctcgaggtt

SEQ ID NO: 58 - DNA, 31 bp, synthetic construct
  source 1..31: mol_type = other DNA
actctcgctctgtagagtagtcgaattcgtt

SEQ ID NO: 59 - DNA, 31 bp, synthetic construct
  source 1..31: mol_type = other DNA
tgctcgcagtatcacaagatctctcgaggtt

SEQ ID NO: 60 - DNA, 31 bp, synthetic construct
  source 1..31: mol_type = other DNA
tgctcgcagtatcacagtagtcgaattcgtt

SEQ ID NO: 61 - DNA, 31 bp, synthetic construct
  source 1..31: mol_type = other DNA
cagtgagagcgcgataagatctctcgaggtt

SEQ ID NO: 62 - DNA, 31 bp, synthetic construct
  source 1..31: mol_type = other DNA
cagtgagagcgcgatagtagtcgaattcgtt

SEQ ID NO: 63 - DNA, 31 bp, synthetic construct
  source 1..31: mol_type = other DNA
tcacactctagagcgaagatctctcgaggtt

SEQ ID NO: 64 - DNA, 31 bp, synthetic construct
  source 1..31: mol_type = other DNA
tcacactctagagcgagtagtcgaattcgtt

SEQ ID NO: 65 - DNA, 31 bp, synthetic construct
  source 1..31: mol_type = other DNA
gcagactctcacacgcagatctctcgaggtt

SEQ ID NO: 66 - DNA, 31 bp, synthetic construct
  source 1..31: mol_type = other DNA
gcagactctcacacgcgtagtcgaattcgtt

SEQ ID NO: 67 - DNA, 31 bp, synthetic construct
  source 1..31: mol_type = other DNA
gtgtgagatatatatcagatctctcgaggtt

SEQ ID NO: 68 - DNA, 31 bp, synthetic construct
  source 1..31: mol_type = other DNA
gtgtgagatatatatcgtagtcgaattcgtt

SEQ ID NO: 69 - DNA, 31 bp, synthetic construct
  source 1..31: mol_type = other DNA
gacagcatctgcgctcagatctctcgaggtt

SEQ ID NO: 70 - DNA, 31 bp, synthetic construct
  source 1..31: mol_type = other DNA
gacagcatctgcgctcgtagtcgaattcgtt

SEQ ID NO: 71 - DNA, 31 bp, synthetic construct
  source 1..31: mol_type = other DNA
ctgcgcagtacgtgcaagatctctcgaggtt

SEQ ID NO: 72 - DNA, 31 bp, synthetic construct
  source 1..31: mol_type = other DNA
ctgcgcagtacgtgcagtagtcgaattcgtt

SEQ ID NO: 73 - DNA, 31 bp, synthetic construct
  source 1..31: mol_type = other DNA
ctgcgtgctctacgacagatctctcgaggtt

SEQ ID NO: 74 - DNA, 31 bp, synthetic construct
  source 1..31: mol_type = other DNA
ctgcgtgctctacgacgtagtcgaattcgtt

SEQ ID NO: 75 - DNA, 31 bp, synthetic construct
  source 1..31: mol_type = other DNA
cacatatcagagtgcgagatctctcgaggtt

SEQ ID NO: 76 - DNA, 31 bp, synthetic construct
  source 1..31: mol_type = other DNA
cacatatcagagtgcggtagtcgaattcgtt

SEQ ID NO: 77 - DNA, 31 bp, synthetic construct
  source 1..31: mol_type = other DNA
acacacagactgtgagagatctctcgaggtt

SEQ ID NO: 78 - DNA, 31 bp, synthetic construct
  source 1..31: mol_type = other DNA
acacacagactgtgaggtagtcgaattcgtt

SEQ ID NO: 79 - DNA, 31 bp, synthetic construct
  source 1..31: mol_type = other DNA
acacatctcgtgagagagatctctcgaggtt

SEQ ID NO: 80 - DNA, 31 bp, synthetic construct
  source 1..31: mol_type = other DNA
acacatctcgtgagaggtagtcgaattcgtt

SEQ ID NO: 81 - DNA, 31 bp, synthetic construct
  source 1..31: mol_type = other DNA
cacgcacacacgcgcgagatctctcgaggtt

SEQ ID NO: 82 - DNA, 31 bp, synthetic construct
  source 1..31: mol_type = other DNA
cacgcacacacgcgcggtagtcgaattcgtt

SEQ ID NO: 83 - DNA, 31 bp, synthetic construct
  source 1..31: mol_type = other DNA
catatatatcagctgtagatctctcgaggtt

SEQ ID NO: 84 - DNA, 31 bp, synthetic construct
  source 1..31: mol_type = other DNA
catatatatcagctgtgtagtcgaattcgtt

SEQ ID NO: 85 - DNA, 31 bp, synthetic construct; identical to SEQ ID NO: 77
  source 1..31: mol_type = other DNA

SEQ ID NO: 86 - DNA, 31 bp, synthetic construct; identical to SEQ ID NO: 78
  source 1..31: mol_type = other DNA

SEQ ID NO: 87 - DNA, 31 bp, synthetic construct
  source 1..31: mol_type = other DNA
tctgtatctctatgtgagatctctcgaggtt

SEQ ID NO: 88 - DNA, 31 bp, synthetic construct
  source 1..31: mol_type = other DNA
tctgtatctctatgtggtagtcgaattcgtt

SEQ ID NO: 89 - DNA, 31 bp, synthetic construct
  source 1..31: mol_type = other DNA
acagtcgagcgctgcgagatctctcgaggtt

SEQ ID NO: 90 - DNA, 31 bp, synthetic construct
  source 1..31: mol_type = other DNA
acagtcgagcgctgcggtagtcgaattcgtt

SEQ ID NO: 91 - DNA, 31 bp, synthetic construct
  source 1..31: mol_type = other DNA
acacacgcgagacagaagatctctcgaggtt

SEQ ID NO: 92 - DNA, 31 bp, synthetic construct
  source 1..31: mol_type = other DNA
acacacgcgagacagagtagtcgaattcgtt

SEQ ID NO: 93 - DNA, 31 bp, synthetic construct
  source 1..31: mol_type = other DNA
acgcgctatctcagagagatctctcgaggtt

SEQ ID NO: 94 - DNA, 31 bp, synthetic construct
  source 1..31: mol_type = other DNA
acgcgctatctcagaggtagtcgaattcgtt

SEQ ID NO: 95 - DNA, 20 bp, synthetic construct
  source 1..20: mol_type = other DNA
aggcagacctcatgctcaat

SEQ ID NO: 96 - DNA, 20 bp, synthetic construct
  source 1..20: mol_type = other DNA
ctccatgcccaccaaagtca

SEQ ID NO: 97 - DNA, 20 bp, synthetic construct
  source 1..20: mol_type = other DNA
gcaagctgaccctgaagttc

SEQ ID NO: 98 - DNA, 20 bp, synthetic construct
  source 1..20: mol_type = other DNA
cttctcgttggggtctttgc

SEQ ID NO: 99 - DNA, 37 bp, synthetic construct
  source 1..37: mol_type = other DNA
ggcagggtgggcagctccaggtttataggctgcaccc

SEQ ID NO: 100 - DNA, 34 bp, synthetic construct
  source 1..34: mol_type = other DNA
tattagccataagactaccaccggtcatggtcga

SEQ ID NO: 101 - DNA, 35 bp, synthetic construct
  source 1..35: mol_type = other DNA
agcaggcatgctggggatatagggtcagctcagtc

SEQ ID NO: 102 - DNA, 33 bp, synthetic construct
  source 1..33: mol_type = other DNA
cagcttcacacaagaatgaccacaccagatgcc